SEQUENCE LISTING (192 sequences)

SEQ ID NO: 1 - DNA, 2127 bp, artificial sequence
  IgFD-4(anti-CD19/CD3) chain-1
gaagttcagttgcagcagtcagggcctgaactcgtgaagccgggtgcctctatgaagatctcttgcaaggcaagcggctattcctttactggctacaccatgaattgggtaaaacaatcccacggcaagaacttggagtggatgggcttgattaacccatacaagggagtgtctacttacaaccagaagtttaaagacaaagccaccttgacagttgacaagagttccagcacagcgtacatggaactcctgtcccttactagcgaagacagcgctgtgtactattgcgcccgatctgggtactacggcgattccgattggtacttcgatgtctggggacaagggactacgcttacagtattctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaagcggaggcgggggaagcggcggcggagggtctggaggagggggcagtgatattcagatgacacagactactagctctctgagcgcaagtttgggcgaccgcgtcacaatctcttgtcgagctagtcaagacattagtaagtacctgaattggtaccaacagaaaccagacggtacagtgaagctgctcatttaccacacaagcagactccacagcggcgtgccttcccggttcagtggctccgggagcggtaccgattatagccttacgatctccaacctcgaacaggaagacatcgctacatacttctgtcagcaaggtaacaccctcccctacacgttcgggggaggcacaaagttggagattaccggaggcggcggttctggtggcggtgggagcggaggcggtgggagcgaagtcaaactccaggaatcaggccctggccttgtcgcccctagtcaaagtctgagcgttacgtgcactgtctcaggtgtttcacttcctgactatggggtatcatggataagacagcctccacgaaaaggtcttgagtggctcggagtcatatggggtagtgagactacttactataacagcgccctgaaatcaagattgaccataatcaaagataatagtaagagtcaggtatttttgaaaatgaactctttgcaaactgatgataccgccatttattactgcgctaaacattactattatggcggttcctatgcgatggactattggggccaaggaacgtccgtaacggtttccagt

SEQ ID NO: 2 - protein, 709 aa, artificial sequence
  IgFD-4(anti-CD19/CD3) chain-1
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - DNA, 1320 bp, artificial sequence
  IgFD-4(anti-CD19/CD3) chain-2
gatatacagatgacccagactacgagcagtttgtctgccagcctgggggatagagtcactattagctgccgggccagccaggatattagaaattatctcaattggtaccaacagaagccagatgggacagtgaaattgctgatatactacacctctcggcttcattcaggggtaccaagcaaattctctggcagtggcagcggaacggattattctcttacgatatcaaatctggaacaggaggacattgcgacgtatttctgccagcaggggaatactctgccatggaccttcgccggggggactaagctcgaaataaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 4 - protein, 440 aa, artificial sequence
  IgFD-4(anti-CD19/CD3) chain-2
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - DNA, 2136 bp, artificial sequence
  IgFD-6(anti-CD3/CD19) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaagcggaggcgggggaagcggcggcggagggtctggaggagggggcagtgatattcagatgacacagactactagctctctgagcgcaagtttgggcgaccgcgtcacaatctcttgtcgagctagtcaagacattagtaagtacctgaattggtaccaacagaaaccagacggtacagtgaagctgctcatttaccacacaagcagactccacagcggcgtgccttcccggttcagtggctccgggagcggtaccgattatagccttacgatctccaacctcgaacaggaagacatcgctacatacttctgtcagcaaggtaacaccctcccctacacgttcgggggaggcacaaagttggagattaccggaggcggcggttctggtggcggtgggagcggaggcggtgggagcgaagtcaaactccaggaatcaggccctggccttgtcgcccctagtcaaagtctgagcgttacgtgcactgtctcaggtgtttcacttcctgactatggggtatcatggataagacagcctccacgaaaaggtcttgagtggctcggagtcatatggggtagtgagactacttactataacagcgccctgaaatcaagattgaccataatcaaagataatagtaagagtcaggtatttttgaaaatgaactctttgcaaactgatgataccgccatttattactgcgctaaacattactattatggcggttcctatgcgatggactattggggccaaggaacgtccgtaacggtttccagt

SEQ ID NO: 6 - protein, 712 aa, artificial sequence
  IgFD-6(anti-CD19/CD3) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 7 - DNA, 1329 bp, artificial sequence
  IgFD-6(anti-CD19/CD3) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 8 - protein, 443 aa, artificial sequence
  IgFD-6(anti-CD19/CD3) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 9 - DNA, 1572 bp, artificial sequence
  IgFD-17(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtaatagtgacccgaaatgtcctctcagtcacgaagggtactgtcttaatgatggggtgtgcatgtatattggaactttggaccgatatgcctgtaattgtgttgtgggctacgtcggtgaacgatgccaataccgagatttgaagtggcttgcactccga

SEQ ID NO: 10 - protein, 524 aa, artificial sequence
  IgFD-17(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSNSDPKCPLSHEGYCLNDGVCMYIGTLDRYACNCVVGYVGERCQYRDLKWLALR

SEQ ID NO: 11 - DNA, 1332 bp, artificial sequence
  IgFD-17(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 12 - protein, 444 aa, artificial sequence
  IgFD-17(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 13 - DNA, 2136 bp, artificial sequence
  IgFD-21(anti-CD3/EGFR) chain-1
gaggtgcagctggtggagagcggcggcggcctggtgcagcccggcggcagcctgaagctgagctgcgccgccagcggcttcaccttcaacaagtacgccatgaactgggtgcgccaggcccccggcaagggcctggagtgggtggcccgcatccgcagcaagtacaacaactacgccacctactacgccgacagcgtgaaggaccgcttcaccatcagccgcgacgacagcaagaacaccgcctacctgcagatgaacaacctgaagaccgaggacaccgccgtgtactactgcgtgcgccacggcaacttcggcaacagctacatcagctactgggcctactggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagggtggcggtggctcgggcggtggtgggtcgggtggcggcggatctcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagc

SEQ ID NO: 14 - protein, 712 aa, artificial sequence
  IgFD-21(anti-CD3/EGFR) chain-1
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGGGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 15 - DNA, 1329 bp, artificial sequence
  IgFD-21(anti-CD3/EGFR) chain-2
cagaccgtggtgacccaggagcccagcctgaccgtgagccccggcggcaccgtgaccctgacctgcggcagcagcaccggcgccgtgaccagcggcaactaccccaactgggtgcagcagaagccaggacaggctcctcgtggtctgatcggaggtaccaagttcctggctcctggtacacctgcacgcttcagcggcagcctgctgggcggcaaggccgccctgaccctgagcggcgtgcagcccgaggacgaggccgagtactactgcgtgctgtggtacagcaaccgctgggtgttcggcggcggcaccaagctgaccgtgctgcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 16 - protein, 443 aa, artificial sequence
  IgFD-21(anti-CD3/EGFR) chain-2
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - DNA, 2136 bp, artificial sequence
  IgFD-18(anti-CD3/EGFR) chain-1
gacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagggtggcggtggctcgggcggtggtgggtcgggtggcggcggatctcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcggaggcagcgggggaagcggcggcggagggtctggaggagggggcagtgaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 18 - protein, 712 aa, artificial sequence
  IgFD-18(anti-CD3/EGFR) chain-1
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGGGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSGGSGGSGGGGSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 19 - DNA, 1353 bp, artificial sequence
  IgFD-5(anti-CD3/CD3) chain-1
gaagttcagttgcagcagtcagggcctgaactcgtgaagccgggtgcctctatgaagatctcttgcaaggcaagcggctattcctttactggctacaccatgaattgggtaaaacaatcccacggcaagaacttggagtggatgggcttgattaacccatacaagggagtgtctacttacaaccagaagtttaaagacaaagccaccttgacagttgacaagagttccagcacagcgtacatggaactcctgtcccttactagcgaagacagcgctgtgtactattgcgcccgatctgggtactacggcgattccgattggtacttcgatgtctggggacaagggactacgcttacagtattctcagcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 20 - protein, 451 aa, artificial sequence
  IgFD-5(anti-CD3/CD3) chain-1
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 21 - DNA, 2094 bp, artificial sequence
  IgFD-5(anti-CD19/CD3) chain-2
gatatacagatgacccagactacgagcagtttgtctgccagcctgggggatagagtcactattagctgccgggccagccaggatattagaaattatctcaattggtaccaacagaagccagatgggacagtgaaattgctgatatactacacctctcggcttcattcaggggtaccaagcaaattctctggcagtggcagcggaacggattattctcttacgatatcaaatctggaacaggaggacattgcgacgtatttctgccagcaggggaatactctgccatggaccttcgccggggggactaagctcgaaataaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaagcggaggcgggggaagcggcggcggagggtctggaggagggggcagtgatattcagatgacacagactactagctctctgagcgcaagtttgggcgaccgcgtcacaatctcttgtcgagctagtcaagacattagtaagtacctgaattggtaccaacagaaaccagacggtacagtgaagctgctcatttaccacacaagcagactccacagcggcgtgccttcccggttcagtggctccgggagcggtaccgattatagccttacgatctccaacctcgaacaggaagacatcgctacatacttctgtcagcaaggtaacaccctcccctacacgttcgggggaggcacaaagttggagattaccggaggcggcggttctggtggcggtgggagcggaggcggtgggagcgaagtcaaactccaggaatcaggccctggccttgtcgcccctagtcaaagtctgagcgttacgtgcactgtctcaggtgtttcacttcctgactatggggtatcatggataagacagcctccacgaaaaggtcttgagtggctcggagtcatatggggtagtgagactacttactataacagcgccctgaaatcaagattgaccataatcaaagataatagtaagagtcaggtatttttgaaaatgaactctttgcaaactgatgataccgccatttattactgcgctaaacattactattatggcggttcctatgcgatggactattggggccaaggaacgtccgtaacggtttccagt

SEQ ID NO: 22 - protein, 698 aa, artificial sequence
  IgFD-5(anti-CD19/CD3) chain-2
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 23 - DNA, 1362 bp, artificial sequence
  IgFD-7(anti-CD19/CD3) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 24 - protein, 454 aa, artificial sequence
  IgFD-7(anti-CD19/CD3) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - DNA, 2103 bp, artificial sequence
  IgFD-7(anti-CD19/CD3) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaagcggaggcgggggaagcggcggcggagggtctggaggagggggcagtgatattcagatgacacagactactagctctctgagcgcaagtttgggcgaccgcgtcacaatctcttgtcgagctagtcaagacattagtaagtacctgaattggtaccaacagaaaccagacggtacagtgaagctgctcatttaccacacaagcagactccacagcggcgtgccttcccggttcagtggctccgggagcggtaccgattatagccttacgatctccaacctcgaacaggaagacatcgctacatacttctgtcagcaaggtaacaccctcccctacacgttcgggggaggcacaaagttggagattaccggaggcggcggttctggtggcggtgggagcggaggcggtgggagcgaagtcaaactccaggaatcaggccctggccttgtcgcccctagtcaaagtctgagcgttacgtgcactgtctcaggtgtttcacttcctgactatggggtatcatggataagacagcctccacgaaaaggtcttgagtggctcggagtcatatggggtagtgagactacttactataacagcgccctgaaatcaagattgaccataatcaaagataatagtaagagtcaggtatttttgaaaatgaactctttgcaaactgatgataccgccatttattactgcgctaaacattactattatggcggttcctatgcgatggactattggggccaaggaacgtccgtaacggtttccagt

SEQ ID NO: 26 - protein, 701 aa, artificial sequence
  IgFD-7(anti-CD19/CD3) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 27 - DNA, 2100 bp, artificial sequence
  IgFD-8(anti-CD19/CD3) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagggtggcggtggctcgggcggtggtgggtcgggtggcggcggatctcaggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagc

SEQ ID NO: 28 - protein, 700 aa, artificial sequence
  IgFD-8(anti-CD19/CD3) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGGGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 29 - DNA, 1365 bp, artificial sequence
  IgFD-11(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 30 - protein, 455 aa, artificial sequence
  IgFD-11(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - DNA, 2103 bp, artificial sequence
  IgFD-11(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagggtggcggtggctcgggcggtggtgggtcgggtggcggcggatctcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagc

SEQ ID NO: 32 - protein, 701 aa, artificial sequence
  IgFD-11(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGGGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 33 - DNA, 1539 bp, artificial sequence
  IgFD-16(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtaatagtgacccgaaatgtcctctcagtcacgaagggtactgtcttaatgatggggtgtgcatgtatattggaactttggaccgatatgcctgtaattgtgttgtgggctacgtcggtgaacgatgccaataccgagatttgaagtggcttgcactccga

SEQ ID NO: 34 - protein, 513 aa, artificial sequence
  IgFD-16(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSNSDPKCPLSHEGYCLNDGVCMYIGTLDRYACNCVVGYVGERCQYRDLKWLALR

SEQ ID NO: 35 - DNA, 1365 bp, artificial sequence
  IgFD-20(anti-CD3/EGFR) chain-1
gaggtgcagctggtggagagcggcggcggcctggtgcagcccggcggcagcctgaagctgagctgcgccgccagcggcttcaccttcaacaagtacgccatgaactgggtgcgccaggcccccggcaagggcctggagtgggtggcccgcatccgcagcaagtacaacaactacgccacctactacgccgacagcgtgaaggaccgcttcaccatcagccgcgacgacagcaagaacaccgcctacctgcagatgaacaacctgaagaccgaggacaccgccgtgtactactgcgtgcgccacggcaacttcggcaacagctacatcagctactgggcctactggggccagggcaccctggtgaccgtgagcagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 36 - protein, 455 aa, artificial sequence
  IgFD-20(anti-CD3/EGFR) chain-1
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - DNA, 2100 bp, artificial sequence
  IgFD-20(anti-CD3/EGFR) chain-2
cagaccgtggtgacccaggagcccagcctgaccgtgagccccggcggcaccgtgaccctgacctgcggcagcagcaccggcgccgtgaccagcggcaactaccccaactgggtgcagcagaagccaggacaggctcctcgtggtctgatcggaggtaccaagttcctggctcctggtacacctgcacgcttcagcggcagcctgctgggcggcaaggccgccctgaccctgagcggcgtgcagcccgaggacgaggccgagtactactgcgtgctgtggtacagcaaccgctgggtgttcggcggcggcaccaagctgaccgtgctgcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagggtggcggtggctcgggcggtggtgggtcgggtggcggcggatctcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagc

SEQ ID NO: 38 - protein, 700 aa, artificial sequence
  IgFD-20(anti-CD3/EGFR) chain-2
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGGGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 39 - DNA, 2145 bp, artificial sequence
  IgFD-22(anti-CD3/BCMA) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgcggcccaggcggccgagctcgtgatgactcagtctccttccaccctgtctgcatctgtaggagacagagtcaccatcacttgccgggcgagtcagggtattagcagctggttagcctggtatcagcagaaaccagggaaagcccctaagctcctgatctatgctgcatccagtctgcaaagtggggtcccatcaaggttcagcggcagtggatctgggacagatttcactctcactatcagcagactgcaacctgaagattttgcaacttactactgtcaacagagttacagtaccccgtacacttttggccaggggaccaagctggagatcaaaggcggctcctctagatcttcctcctctggtggcggcggctcgggcggtggtgggcaggtgcagctggtgcagtctggggctgaggtgaaggagcctggggcctcagtgaaggtttcctgcaaggcatctggatacgccttcagcaaacactttatccactgggtgcgacaggcccctggacaggggcttgaatggctggcaatgatcaaccctactgatgataacacacactctgctcagatgttccaagacagagtcaccgtgaccaaggacatgtccacgagcatggtctacatggagctgagcagcctaagatctgacgacacggccgtttattactgtgccagagatcgcacttctaatgttggtggttatttcccacccctctttgacttctggggccagggaacgctggtcaccgtctcctca

SEQ ID NO: 40 - protein, 715 aa, artificial sequence
  IgFD-22(anti-CD3/BCMA) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSAAQAAELVMTQSPSTLSASVGDRVTITCRASQGISSWLAWYQQKPGKAPKLLIYAASSLQSGVPSRFSGSGSGTDFTLTISRLQPEDFATYYCQQSYSTPYTFGQGTKLEIKGGSSRSSSSGGGGSGGGGQVQLVQSGAEVKEPGASVKVSCKASGYAFSKHFIHWVRQAPGQGLEWLAMINPTDDNTHSAQMFQDRVTVTKDMSTSMVYMELSSLRSDDTAVYYCARDRTSNVGGYFPPLFDFWGQGTLVTVSS

SEQ ID NO: 41 - DNA, 2097 bp, artificial sequence
  IgFD-23(anti-CD3/CLL-1) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgaagtgcagctggtggagtctgggggaggcctggtgaagcctggggggtctctgagactctcctgtgcagtctcgggattcagcttcagtggcaatagcatgagctgggtccgccaggctccagggaaggggctggagtgggtctcatacattgatcgtgatggtagtcacatattctacacagactcagtgaggggccgattcaccatctccagagacgacgccaagaactcactgtatctccaaatgaacagcctgagaacagaggacacggctgtgtattactgtgtagcggaaatcggggcacgggacttcctgcactggggccagggcaccctggtcaccgtctcctcaggaggcgggggaagcggcggcggagggtctggaggagggggcagtgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatggtgcatccagtttgcaaagtggggtcccatcaaggttcagtggcagtggatctgggacagatttcactctcaccatcagcagcctgcagcctgaagattttgcaacttattactgccaacagtataatagttattggacgttcggccaagggaccaaagtggatatcaaa

SEQ ID NO: 42 - protein, 699 aa, artificial sequence
  IgFD-23(anti-CD3/CLL-1) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSEVQLVESGGGLVKPGGSLRLSCAVSGFSFSGNSMSWVRQAPGKGLEWVSYIDRDGSHIFYTDSVRGRFTISRDDAKNSLYLQMNSLRTEDTAVYYCVAEIGARDFLHWGQGTLVTVSSGGGGSGGGGSGGGGSDIQMTQSPSSLSASVGDRVTITCRASQSISSYLNWYQQKPGKAPKLLIYGASSLQSGVPSRFSGSGSGTDFTLTISSLQPEDFATYYCQQYNSYWTFGQGTKVDIK

SEQ ID NO: 43 - DNA, 1362 bp, artificial sequence
  IgFD-25(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 44 - protein, 454 aa, artificial sequence
  IgFD-25(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - DNA, 2100 bp, artificial sequence
  IgFD-25(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagggtggcggtggctcgggcggtggtgggtcgggtggcggcggatctcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagc

SEQ ID NO: 46 - protein, 700 aa, artificial sequence
  IgFD-25(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGGGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 47 - DNA, 1365 bp, artificial sequence
  IgFD-26(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtacaccctgcccccatgccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 48 - protein, 455 aa, artificial sequence
  IgFD-26(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - DNA, 2103 bp, artificial sequence
  IgFD-26(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatagagaaaaccatctccaaagccaaagggcagccccgagaaccacaggtgtgcaccctgcccccatcccgggaggagatgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagggtggcggtggctcgggcggtggtgggtcgggtggcggcggatctcaggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagc

SEQ ID NO: 50 - protein, 701 aa, artificial sequence
  IgFD-26(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGGGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 51 - DNA, 2097 bp, artificial sequence
  IgFD-28(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactggagatcaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagggtggcggtggctcgggcggtggtgggtcgggtggcggcggatctcaggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagc

SEQ ID NO: 52 - protein, 699 aa, artificial sequence
  IgFD-28(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGGGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 53 - DNA, 1431 bp, artificial sequence
  IgFD-31(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcggcggaagcggagcaaagctcgccgcactgaaagccaagctggccgctctgaaggggggtggcggaagcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 54 - protein, 477 aa, artificial sequence
  IgFD-31(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCGGSGAKLAALKAKLAALKGGGGSDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 55 - DNA, 2169 bp, artificial sequence
  IgFD-31(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtggcggaggtgggagtgaactggccgcactggaagctgagctggctgccctcgaagctggaggctctggagacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagggtggcggtggctcgggcggtggtgggtcgggtggcggcggatctcaggtgcagctgcaggagagcggccccggcctggtgaagcccagcgagaccctgagcctgacctgcaccgtgagcggcggcagcgtgagcagcggcgactactactggacctggatccgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagc

SEQ ID NO: 56 - protein, 723 aa, artificial sequence
  IgFD-31(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSELAALEAELAALEAGGSGDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGGGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 57 - DNA, 2103 bp, artificial sequence
  IgFD-19(anti-CD3/EGFR) chain-2
gacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagggtggcggtggctcgggcggtggtgggtcgggtggcggcggatctcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcggaggcagcgggggaagcggcggcggagggtctggaggagggggcagtcaagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 58 - protein, 701 aa, artificial sequence
  IgFD-19(anti-CD3/EGFR) chain-2
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGGGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSGGSGGSGGGGSGGGGSQAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 59 - DNA, 1767 bp, artificial sequence
  IgFD-9(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagc

SEQ ID NO: 60 - protein, 589 aa, artificial sequence
  IgFD-9(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 61 - DNA, 1698 bp, artificial sequence
  IgFD-9(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaag

SEQ ID NO: 62 - protein, 566 aa, artificial sequence
  IgFD-9(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIK

SEQ ID NO: 63 - DNA, 1770 bp, artificial sequence
  IgFD-12(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagc

SEQ ID NO: 64 - protein, 590 aa, artificial sequence
  IgFD-12(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 65 - DNA, 1701 bp, artificial sequence
  IgFD-12(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaag

SEQ ID NO: 66 - protein, 567 aa, artificial sequence
  IgFD-12(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIK

SEQ ID NO: 67 - DNA, 1767 bp, artificial sequence
  IgFD-29(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagc

SEQ ID NO: 68 - protein, 589 aa, artificial sequence
  IgFD-29(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 69 - DNA, 1698 bp, artificial sequence
  IgFD-29(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaag

SEQ ID NO: 70 - protein, 566 aa, artificial sequence
  IgFD-29(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIK

SEQ ID NO: 71 - DNA, 1731 bp, artificial sequence
  IgFD-10(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaag

SEQ ID NO: 72 - protein, 577 aa, artificial sequence
  IgFD-10(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIK

SEQ ID NO: 73 - DNA, 1734 bp, artificial sequence
  IgFD-10(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagc

SEQ ID NO: 74 - protein, 578 aa, artificial sequence
  IgFD-10(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 75 - DNA, 1734 bp, artificial sequence
  IgFD-13(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaag

SEQ ID NO: 76 - protein, 578 aa, artificial sequence
  IgFD-13(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIK

SEQ ID NO: 77 - DNA, 1737 bp, artificial sequence
  IgFD-13(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagc

SEQ ID NO: 78 - protein, 579 aa, artificial sequence
  IgFD-13(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 79 - DNA, 1731 bp, artificial sequence
  IgFD-30(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaag

SEQ ID NO: 80 - protein, 577 aa, artificial sequence
  IgFD-30(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIK

SEQ ID NO: 81 - DNA, 1734 bp, artificial sequence
  IgFD-30(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagc

SEQ ID NO: 82 - protein, 578 aa, artificial sequence
  IgFD-30(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 83 - DNA, 2094 bp, artificial sequence
  IgFD-14(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacaca

SEQ ID NO: 84 - protein, 698 aa, artificial sequence
  IgFD-14(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 85 - DNA, 2022 bp, artificial sequence
  IgFD-14(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 86 - protein, 674 aa, artificial sequence
  IgFD-14(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 87 - DNA, 2055 bp, artificial sequence
  IgFD-15(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgt

SEQ ID NO: 88 - protein, 685 aa, artificial sequence
  IgFD-15(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - DNA, 2061 bp, artificial sequence
  IgFD-15(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggaggcagcgggggaagcggcggcggagggtctggaggagggggcagtcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacaca

SEQ ID NO: 90 - protein, 687 aa, artificial sequence
  IgFD-15(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKGGSGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHT

SEQ ID NO: 91 - DNA, 2178 bp, artificial sequence
  IgFD-1(anti-CD19/CD3) chain-1
gatattcagatgacacagactactagctctctgagcgcaagtttgggcgaccgcgtcacaatctcttgtcgagctagtcaagacattagtaagtacctgaattggtaccaacagaaaccagacggtacagtgaagctgctcatttaccacacaagcagactccacagcggcgtgccttcccggttcagtggctccgggagcggtaccgattatagccttacgatctccaacctcgaacaggaagacatcgctacatacttctgtcagcaaggtaacaccctcccctacacgttcgggggaggcacaaagttggagattaccggaggcggcggttctggtggcggtgggagcggaggcggtgggagcgaagtcaaactccaggaatcaggccctggccttgtcgcccctagtcaaagtctgagcgttacgtgcactgtctcaggtgtttcacttcctgactatggggtatcatggataagacagcctccacgaaaaggtcttgagtggctcggagtcatatggggtagtgagactacttactataacagcgccctgaaatcaagattgaccataatcaaagataatagtaagagtcaggtatttttgaaaatgaactctttgcaaactgatgataccgccatttattactgcgctaaacattactattatggcggttcctatgcgatggactattggggccaaggaacgtccgtaacggtttccagtgggggtggcgaagcagctgctaaagaggcagccgcaaaggaagcagctgcaaaggcaggaggcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccagtgcatcaacaaagggaccaagcgtgtttccactggccccctcaagcaagagcacctccggagggacagccgctctgggatgtctggtgaaagactacttccccgagcctgtgactgtctcttggaatagtggcgctctgacctccggggtgcacacatttccagcagtcctgcagtcctctggactgtattctctgagttcagtggtcaccgtgcccagctcctctctgggcactcagacctacatctgcaatgtcaaccataagcctagtaacacaaaagtggataagaaagtcgaaccaaagagctgtgacaagacccacaccggtggcggagggagcgaagttcagttgcagcagtcagggcctgaactcgtgaagccgggtgcctctatgaagatctcttgcaaggcaagcggctattcctttactggctacaccatgaattgggtaaaacaatcccacggcaagaacttggagtggatgggcttgattaacccatacaagggagtgtctacttacaaccagaagtttaaagacaaagccaccttgacagttgacaagagttccagcacagcgtacatggaactcctgtcccttactagcgaagacagcgctgtgtactattgcgcccgatctgggtactacggcgattccgattggtacttcgatgtctggggacaagggactacgcttacagtattctca

SEQ ID NO: 92 - protein, 726 aa, artificial sequence
  IgFD-1(anti-CD19/CD3) chain-1
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGEAAAKEAAAKEAAAKAGGDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGGGSEVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 93 - DNA, 1332 bp, artificial sequence
  IgFD-1(anti-CD19/CD3) chain-2
gacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtggtggcggagggagcgatatacagatgacccagactacgagcagtttgtctgccagcctgggggatagagtcactattagctgccgggccagccaggatattagaaattatctcaattggtaccaacagaagccagatgggacagtgaaattgctgatatactacacctctcggcttcattcaggggtaccaagcaaattctctggcagtggcagcggaacggattattctcttacgatatcaaatctggaacaggaggacattgcgacgtatttctgccagcaggggaatactctgccatggaccttcgccggggggactaagctcgaaataaaa

SEQ ID NO: 94 - protein, 444 aa, artificial sequence
  IgFD-1(anti-CD19/CD3) chain-2
DKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGGGSDIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 95 - DNA, 2223 bp, artificial sequence
  IgFD-2(anti-CD19/CD3) chain-1
gatattcagatgacacagactactagctctctgagcgcaagtttgggcgaccgcgtcacaatctcttgtcgagctagtcaagacattagtaagtacctgaattggtaccaacagaaaccagacggtacagtgaagctgctcatttaccacacaagcagactccacagcggcgtgccttcccggttcagtggctccgggagcggtaccgattatagccttacgatctccaacctcgaacaggaagacatcgctacatacttctgtcagcaaggtaacaccctcccctacacgttcgggggaggcacaaagttggagattaccggaggcggcggttctggtggcggtgggagcggaggcggtgggagcgaagtcaaactccaggaatcaggccctggccttgtcgcccctagtcaaagtctgagcgttacgtgcactgtctcaggtgtttcacttcctgactatggggtatcatggataagacagcctccacgaaaaggtcttgagtggctcggagtcatatggggtagtgagactacttactataacagcgccctgaaatcaagattgaccataatcaaagataatagtaagagtcaggtatttttgaaaatgaactctttgcaaactgatgataccgccatttattactgcgctaaacattactattatggcggttcctatgcgatggactattggggccaaggaacgtccgtaacggtttccagtgggggtggcgaagcagctgctaaagaggcagccgcaaaggaagcagctgcaaaggcaggaggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccagtgcatcaacaaagggaccaagcgtgtttccactggccccctcaagcaagagcacctccggagggacagccgctctgggatgtctggtgaaagactacttccccgagcctgtgactgtctcttggaatagtggcgctctgacctccggggtgcacacatttccagcagtcctgcagtcctctggactgtattctctgagttcagtggtcaccgtgcccagctcctctctgggcactcagacctacatctgcaatgtcaaccataagcctagtaacacaaaagtggataagaaagtcgaaccaaagagctgtgacaagacccacaccggaggcagcgggggaagcggcggcggagggtctggaggagggggcagtgaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctct

SEQ ID NO: 96 - protein, 741 aa, artificial sequence
  IgFD-2(anti-CD19/CD3) chain-1
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGEAAAKEAAAKEAAAKAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSGGSGGGGSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSS

SEQ ID NO: 97 - DNA, 1377 bp, artificial sequence
  IgFD-2(anti-CD19/CD3) chain-2
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtggaggcagcgggggaagcggcggcggagggtctggaggagggggcagtcaagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaa

SEQ ID NO: 98 - protein, 459 aa, artificial sequence
  IgFD-2(anti-CD19/CD3) chain-2
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSGGGGSGGGGSQAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLK

SEQ ID NO: 99 - DNA, 2178 bp, artificial sequence
  IgFD-3(anti-CD19/CD3) chain-1
gatattcagatgacacagactactagctctctgagcgcaagtttgggcgaccgcgtcacaatctcttgtcgagctagtcaagacattagtaagtacctgaattggtaccaacagaaaccagacggtacagtgaagctgctcatttaccacacaagcagactccacagcggcgtgccttcccggttcagtggctccgggagcggtaccgattatagccttacgatctccaacctcgaacaggaagacatcgctacatacttctgtcagcaaggtaacaccctcccctacacgttcgggggaggcacaaagttggagattaccggaggcggcggttctggtggcggtgggagcggaggcggtgggagcgaagtcaaactccaggaatcaggccctggccttgtcgcccctagtcaaagtctgagcgttacgtgcactgtctcaggtgtttcacttcctgactatggggtatcatggataagacagcctccacgaaaaggtcttgagtggctcggagtcatatggggtagtgagactacttactataacagcgccctgaaatcaagattgaccataatcaaagataatagtaagagtcaggtatttttgaaaatgaactctttgcaaactgatgataccgccatttattactgcgctaaacattactattatggcggttcctatgcgatggactattggggccaaggaacgtccgtaacggtttccagtgggggtggcgaagcagctgctaaagaggcagccgcaaaggaagcagctgcaaaggcaggaggcgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatccagtgcatcaacaaagggaccaagcgtgtttccactggccccctcaagcaagagcacctccggagggacagccgctctgggatgtctggtgaaagactacttccccgagcctgtgactgtctcttggaatagtggcgctctgacctccggggtgcacacatttccagcagtcctgcagtcctctggactgtattctctgagttcagtggtcaccgtgcccagctcctctctgggcactcagacctacatctgcaatgtcaaccataagcctagtaacacaaaagtggataagaaagtcgaaccaaagagctgtgacaagacccacaccggaggcagcgggggaagcggcggcggagggtctggaggagggggcagtcaagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaa

SEQ ID NO: 100 - protein, 726 aa, artificial sequence
  IgFD-3(anti-CD19/CD3) chain-1
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGGGEAAAKEAAAKEAAAKAGGDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTGGSGGSGGGGSGGGGSQAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLK

SEQ ID NO: 101 - DNA, 1422 bp, artificial sequence
  IgFD-3(anti-CD19/CD3) chain-2
gacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacgccagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtggaggcagcgggggaagcggcggcggagggtctggaggagggggcagtgaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctct

SEQ ID NO: 102 - protein, 474 aa, artificial sequence
  IgFD-3(anti-CD19/CD3) chain-2
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGSGGGGSGGGGSEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSS

SEQ ID NO: 103 - DNA, 1737 bp, artificial sequence
  IgFD-32(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgctagtacgaaaggtccgcaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 104 - protein, 579 aa, artificial sequence
  IgFD-32(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - DNA, 1695 bp, artificial sequence
  IgFD-32(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacagccgaaggccgcaccttctgtgacactgttcccacctgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaatgataa

SEQ ID NO: 106 - protein, 563 aa, artificial sequence
  IgFD-32(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKQPKAAPSVTLFPPDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 107 - DNA, 1737 bp, artificial sequence
  IgFD-33(anti-CD3/EGFR) chain-1
caggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgctagtacgaaaggtccggaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 108 - protein, 579 aa, artificial sequence
  IgFD-33(anti-CD3/EGFR) chain-1
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - DNA, 1686 bp, artificial sequence
  IgFD-33(anti-CD3/EGFR) chain-2
gacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagactgttgctgcaccgtcagtgttcatcttccctccacaagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 110 - protein, 562 aa, artificial sequence
  IgFD-33(anti-CD3/EGFR) chain-2
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKTVAAPSVFIFPPQAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 111 - DNA, 2049 bp, artificial sequence
  IgFD-34(anti-CD3/EGFR) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcatcaacaaagggaccaagcgtgtttccactggccccctcaagcaagagcacctccggagggacagccgctctgggatgtctggtgaaagactacttccccgagcctgtgactgtctcttggaatagtggcgctctgacctccggggtgcacacatttccagcagtcctgcagtcctctggactgtattctctgagttcagtggtcaccgtgcccagctcctctctgggcactcagacctacatctgcaatgtcaaccataagcctagtaacacaaaagtggataagaaagtcgaaccaaagagctgtcccaacctgctgggcggcccccaggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 112 - protein, 683 aa, artificial sequence
  IgFD-34(anti-CD3/EGFR) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCPNLLGGPQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - DNA, 2010 bp, artificial sequence
  IgFD-34(anti-CD3/EGFR) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgtggcggcagcggcggcggcggcagcggcggcggcggcagcgacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 114 - protein, 670 aa, artificial sequence
  IgFD-34(anti-CD3/EGFR) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGGGSGGGGSDIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 115 - DNA, 2046 bp, artificial sequence
  IgFD-35(anti-CD3/EGFR) chain-1
caggtgcagctgcaggagagcggccccggcctggtgaagcccagcgagaccctgagcctgacctgcaccgtgagcggcggcagcgtgagcagcggcgactactactggacctggattcgccagagccccggcaagggcctggagtggatcggccacatctactacagcggcaacaccaactacaaccccagcctgaagagccgcctgaccatcagcatcgacaccagcaagacccagttcagcctgaagctgagcagcgtgaccgccgccgacaccgccatctactactgcgtgcgcgaccgcgtgaccggcgccttcgacatctggggccagggcaccatggtgactgtgtctagcgcatcaacaaagggaccaagcgtgtttccactggccccctcaagcaagagcacctccggagggacagccgctctgggatgtctggtgaaagactacttccccgagcctgtgactgtctcttggaatagtggcgctctgacctccggggtgcacacatttccagcagtcctgcagtcctctggactgtattctctgagttcagtggtcaccgtgcccagctcctctctgggcactcagacctacatctgcaatgtcaaccataagcctagtaacacaaaagtggataagaaagtcgaaccaaagagctgtgctagtacgaaaggtccggaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 116 - protein, 682 aa, artificial sequence
  IgFD-35(anti-CD3/EGFR) chain-1
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCASTKGPEVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - DNA, 2010 bp, artificial sequence
  IgFD-35(anti-CD3/EGFR) chain-2
gacatccagatgacccagagccccagcagcctgagcgccagcgtgggcgaccgcgtgaccatcacctgccaggccagccaggacatcagcaactacctgaactggtaccagcagaagcccggcaaggcccccaagctgctgatctacgacgccagcaacctggagaccggcgtgcccagccgcttcagcggcagcggcagcggcaccgacttcaccttcaccatcagcagcctgcagcccgaggacatcgccacctacttctgccagcacttcgaccacctgcccctggccttcggcggcggcaccaaggtggagatcaagcgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtggcggcagcggcggcggcggcagcggcggcggcggcagccaagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgaactgtggctgcaccatctgtcttcatcttcccgccatctgatgagcagttgaaatctggaactgcctctgtcgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgtcctcgcccgtcacaaagagcttcaacaggggagagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 118 - protein, 670 aa, artificial sequence
  IgFD-35(anti-CD3/EGFR) chain-2
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECGGSGGGGSGGGGSQAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 119 - DNA, 1329 bp, artificial sequence
  IgFD-36(anti-CD3) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 120 - protein, 443 aa, artificial sequence
  IgFD-36(anti-CD3) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 121 - DNA, 1848 bp, artificial sequence
  IgFD-37(anti-CD3/MICA) chain-1
gaggtgcagctggtggagtctgggggaggcttggtccagcctggggggtccctgcgtctctcctgtgcagcctctggattcaccttcaatacctacgccatgaactgggtccgccaggctccgggaaagggtttggaatgggttgctcgcattcgcagtaaatataataattatgcaacatattatgccgattcagtgaaagaccgtttcaccatctcccgcgatgattcaaagaacacgctctatctgcaaatgaacagcctgcgcgctgaggacacggccgtgtattactgcgtgcgtcatggcaactttggtaattcttatgtgagctggttcgcctactggggccagggaaccctggtcaccgtcagctctgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgaacccaaatcttgcgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtacaccctgcctccatgccgggatgagctgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaggcggaagcggagcaaagctcgccgcactgaaagccaagctggccgctctgaagggaggtggcgggagcttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcgcaccgtg

SEQ ID NO: 122 - protein, 616 aa, artificial sequence
  IgFD-37(anti-CD3/MICA) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGKGGSGAKLAALKAKLAALKGGGGSFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 123 - DNA, 1815 bp, artificial sequence
  IgFD-37(anti-CD3/MICA) chain-2
caagctgttgtgacccaagaaccgagtctgaccgtgtctccgggcggcaccgttacgctgacctgtcgctcctctaccggcgcagtcaccacgagcaactatgcaaattgggtgcagcaaaaaccggatcatctgtttcgtggcctgattggcggtacgaacaaacgtgcgccggggaccccggcacgtttttcgggcagcctgctgggcgataaagcagcactgaccatcagtggtgcgcagccggaagatgaagcagaatattactgcgctctgtggtatagcaacctgtgggtctttggcggtggcacgaaactgaccgttctgaaacgcacagtggcagcccccagcgtcttcatttttcccccttccgatgaacagctgaagtccggcactgcttctgtggtctgtctgctgaacaatttctatcccagagaggccaaggtgcagtggaaagtggacaacgctctgcagtccggcaacagccaggagagtgtgaccgaacaggatagtaaggacagcacatattctctgtctagtaccctgacactgagtaaggcagattacgagaagcacaaagtgtatgcctgcgaagtcactcatcagggactgtcaagccccgtgaccaagagcttcaaccggggcgagtgtgacaaaactcacacatgcccaccgtgcccagcacctccagtcgccggaccgtcagtcttcctcttccctccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaaggcctcccaagctccatcgagaaaaccatctccaaagccaaagggcagccccgagaaccacaggtgtgcaccctgcctccatcccgggatgagctgaccaagaaccaggtcagcctgtcctgcgcggtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccactacacgcagaagagcctctccctgtctccgggtaaaggcggaagcggagcaaagctcgccgcactgaaagccaagctggccgctctgaagggaggtggcgggagcttcctgaacagcctgttcaaccaggaggtgcagatccccctgaccgagagctactgcggcccctgccccaagaactggatctgctacaagaacaactgctaccagttcttcgacgagagcaagaactggtacgagagccaggccagctgcatgagccagaacgccagcctgctgaaggtgtacagcaaggaggaccaggacctgctgaagctggtgaagagctaccactggatgggcctggtgcacatccccaccaacggcagctggcagtgggaggacggcagcatcctgagccccaacctgctgaccatcatcgagatgcagaagggcgactgcgccctgtacgccagcagcttcaagggctacatcgagaactgcagcacccccaacacctacatctgcatgcagcgcaccgtg

SEQ ID NO: 124 - protein, 605 aa, artificial sequence
  IgFD-37(anti-CD3/MICA) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGSGAKLAALKAKLAALKGGGGSFLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 125 - protein, 228 aa, artificial sequence
  Fab1H(anti-CD3 VH-1-CH1)
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 126 - protein, 217 aa, artificial sequence
  Fab1L(anti-CD3 VL-1-CL)
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 127 - protein, 222 aa, artificial sequence
  Fab2H(anti-EGFR VH-CH1)
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 128 - protein, 214 aa, artificial sequence
  Fab2L(anti-EGFR VL-CL)
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 129 - protein, 225 aa, artificial sequence
  Fab3H(anti-CD3 VH-3-CH1)
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 130 - protein, 214 aa, artificial sequence
  Fab3L(anti-CD3 VL-3-CL)
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 228 aa, artificial sequence
  Fab4H(anti-CD3 VH-4-CH1)
EVQLVESGGGLVQPGGSLKLSCAASGFTFNKYAMNWVRQAPGKGLEWVARIRSKYNNYATYYADSVKDRFTISRDDSKNTAYLQMNNLKTEDTAVYYCVRHGNFGNSYISYWAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 132 - protein, 216 aa, artificial sequence
  Fab4L(anti-CD3 VL-4-CL)
QTVVTQEPSLTVSPGGTVTLTCGSSTGAVTSGNYPNWVQQKPGQAPRGLIGGTKFLAPGTPARFSGSLLGGKAALTLSGVQPEDEAEYYCVLWYSNRWVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 125 aa, artificial sequence
  Fv1(anti-CD3 Fv) chain-1
EVQLVESGGGLVQPGGSLRLSCAASGFTFNTYAMNWVRQAPGKGLEWVARIRSKYNNYATYYADSVKDRFTISRDDSKNTLYLQMNSLRAEDTAVYYCVRHGNFGNSYVSWFAYWGQGTLVTVSS

SEQ ID NO: 134 - protein, 110 aa, artificial sequence
  Fv1(anti-CD3 Fv) chain-2
QAVVTQEPSLTVSPGGTVTLTCRSSTGAVTTSNYANWVQQKPDHLFRGLIGGTNKRAPGTPARFSGSLLGDKAALTISGAQPEDEAEYYCALWYSNLWVFGGGTKLTVLK

SEQ ID NO: 135 - protein, 119 aa, artificial sequence [sequence omitted]

SEQ ID NO: 136 - protein, 107 aa, artificial sequence [sequence omitted]

SEQ ID NO: 137 - protein, 122 aa, artificial sequence
  Fv3(anti-CD3 -3 Fv) chain-1
EVQLQQSGPELVKPGASMKISCKASGYSFTGYTMNWVKQSHGKNLEWMGLINPYKGVSTYNQKFKDKATLTVDKSSSTAYMELLSLTSEDSAVYYCARSGYYGDSDWYFDVWGQGTTLTVFS

SEQ ID NO: 138 - protein, 107 aa, artificial sequence
  Fv3(anti-CD3 -3 Fv) chain-2
DIQMTQTTSSLSASLGDRVTISCRASQDIRNYLNWYQQKPDGTVKLLIYYTSRLHSGVPSKFSGSGSGTDYSLTISNLEQEDIATYFCQQGNTLPWTFAGGTKLEIK

SEQ ID NO: 139 - protein, 242 aa, artificial sequence
  scfv5(anti-CD19 scfv)
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 140 - protein, 255 aa, artificial sequence
  scfv6(anti-BCMA scfv)
AAQAAELVMTQSPSTLSASVGDRVTITCRASQGISSWLAWYQQKPGKAPKLLIYAASSLQSGVPSRFSGSGSGTDFTLTISRLQPEDFATYYCQQSYSTPYTFGQGTKLEIKGGSSRSSSSGGGGSGGGGQVQLVQSGAEVKEPGASVKVSCKASGYAFSKHFIHWVRQAPGQGLEWLAMINPTDDNTHSAQMFQDRVTVTKDMSTSMVYMELSSLRSDDTAVYYCARDRTSNVGGYFPPLFDFWGQGTLVTVSS

SEQ ID NO: 141 - protein, 239 aa, artificial sequence
  scfv7(anti-CLL-1 scfv)
EVQLVESGGGLVKPGGSLRLSCAVSGFSFSGNSMSWVRQAPGKGLEWVSYIDRDGSHIFYTDSVRGRFTISRDDAKNSLYLQMNSLRTEDTAVYYCVAEIGARDFLHWGQGTLVTVSSGGGGSGGGGSGGGGSDIQMTQSPSSLSASVGDRVTITCRASQSISSYLNWYQQKPGKAPKLLIYGASSLQSGVPSRFSGSGSGTDFTLTISSLQPEDFATYYCQQYNSYWTFGQGTKVDIK

SEQ ID NO: 142 - protein, 241 aa, artificial sequence
  scfv2(anti-EGFR scfv)
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKGGGGSGGGGSGGGGSQVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSS

SEQ ID NO: 143 - protein, 227 aa, artificial sequence
  FcV1
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 144 - protein, 226 aa, artificial sequence
  FcV2
DKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 227 aa, artificial sequence
  FcV3
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPCREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 146 - protein, 227 aa, artificial sequence
  FcV4
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVCTLPPSREEMTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 147 - protein, 226 aa, artificial sequence
  FcV5
DKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 226 aa, artificial sequence
  FcV6
DKTHTCPPCPAPPVAGPSVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 149 - protein, 227 aa, artificial sequence
  FcWT
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 150 - protein, 54 aa, artificial sequence
  EGF4
SNSDPKCPLSHEGYCLNDGVCMYIGTLDRYACNCVVGYVGERCQYRDLKWLALR

SEQ ID NO: 151 - protein, 139 aa, artificial sequence
  NKG2D
FLNSLFNQEVQIPLTESYCGPCPKNWICYKNNCYQFFDESKNWYESQASCMSQNASLLKVYSKEDQDLLKLVKSYHWMGLVHIPTNGSWQWEDGSILSPNLLTIIEMQKGDCALYASSFKGYIENCSTPNTYICMQRTV

SEQ ID NO: 152 - protein, 5 aa, artificial sequence
  Linker
GGGGS

SEQ ID NO: 153 - protein, 7 aa, artificial sequence
  Linker
GGGGSGS

SEQ ID NO: 154 - protein, 8 aa, artificial sequence
  Linker
GGGGSGGS

SEQ ID NO: 155 - protein, 10 aa, artificial sequence
  Linker
GGGGSGGGGS

SEQ ID NO: 156 - protein, 15 aa, artificial sequence
  Linker
GGGGSGGGGSGGGGS

SEQ ID NO: 157 - protein, 16 aa, artificial sequence
  Linker
AKTTPKLEEGEFSEAR

SEQ ID NO: 158 - protein, 17 aa, artificial sequence
  Linker
AKTTPKLEEGEFSEARV

SEQ ID NO: 159 - protein, 9 aa, artificial sequence
  Linker
AKTTPKLGG

SEQ ID NO: 160 - protein, 10 aa, artificial sequence
  Linker
SAKTTPKLGG

SEQ ID NO: 161 - protein, 10 aa, artificial sequence
  Linker
SAKTTPKLGG

SEQ ID NO: 162 - protein, 6 aa, artificial sequence
  Linker
SAKTTP

SEQ ID NO: 163 - protein, 10 aa, artificial sequence
  Linker
SAKTTPKLGG

SEQ ID NO: 164 - protein, 6 aa, artificial sequence
  Linker
RADAAP

SEQ ID NO: 165 - protein, 9 aa, artificial sequence
  Linker
RADAAPTVS

SEQ ID NO: 166 - protein, 12 aa, artificial sequence
  Linker
RADAAAAGGPGS

SEQ ID NO: 167 - protein, 27 aa, artificial sequence
  Linker
RADAAAAGGGGSGGGGSGGGGSGGGGS

SEQ ID NO: 168 - protein, 18 aa, artificial sequence
  Linker
SAKTTPKLEEGEFSEARV

SEQ ID NO: 169 - protein, 5 aa, artificial sequence
  Linker
ADAAP

SEQ ID NO: 170 - protein, 12 aa, artificial sequence
  Linker
ADAAPTVSIFPP

SEQ ID NO: 171 - protein, 5 aa, artificial sequence
  Linker
TVAAP

SEQ ID NO: 172 - protein, 12 aa, artificial sequence
  Linker
TVAAPSVFIFPP

SEQ ID NO: 173 - protein, 6 aa, artificial sequence
  Linker
QPKAAP

SEQ ID NO: 174 - protein, 13 aa, artificial sequence
  Linker
QPKAAPSVTLFPP

SEQ ID NO: 175 - protein, 6 aa, artificial sequence
  Linker
AKTTPP

SEQ ID NO: 176 - protein, 13 aa, artificial sequence
  Linker
AKTTPPSVTPLAP

SEQ ID NO: 177 - protein, 6 aa, artificial sequence
  Linker
AKTTAP

SEQ ID NO: 178 - protein, 13 aa, artificial sequence
  Linker
AKTTAPSVYPLAP

SEQ ID NO: 179 - protein, 6 aa, artificial sequence
  Linker
ASTKGP

SEQ ID NO: 180 - protein, 13 aa, artificial sequence
  Linker
ASTKGPSVFPLAP

SEQ ID NO: 181 - protein, 15 aa, artificial sequence
  Linker
GENKVEYAPALMALS

SEQ ID NO: 182 - protein, 15 aa, artificial sequence
  Linker
GPAKELTPLKEAKVS

SEQ ID NO: 183 - protein, 15 aa, artificial sequence
  Linker
GHEAAAVMQVQYPAS

SEQ ID NO: 184 - protein, 23 aa, artificial sequence
  Linker
GGSGAKLAALKAKLAALKGGGGS

SEQ ID NO: 185 - protein, 23 aa, artificial sequence
  Linker
GGGGSELAALEAELAALEAGGSG

SEQ ID NO: 186 - protein, 16 aa, artificial sequence
  linker
SGGGGSGGGGSGGGGS

SEQ ID NO: 187 - protein, 15 aa, artificial sequence
  linker
GGSGGSGGGGSGGGG

SEQ ID NO: 188 - protein, 16 aa, artificial sequence
  linker
GGSGGSGGGGSGGGGS

SEQ ID NO: 189 - protein, 23 aa, artificial sequence
  linker
APATSLQSGQLGFQCGELCSASA

SEQ ID NO: 190 - protein, 7 aa, artificial sequence
  linker
PNLLGGP

SEQ ID NO: 191 - protein, 21 aa, artificial sequence
  linker
GGGEAAAKEAAAKEAAAKAGG

SEQ ID NO: 192 - protein, 13 aa, Artificial Sequence
  Linker
ASTKGPSVFPLAP

(19) United States
(12) Patent Application Publication
(10) Pub. No.: US 2022/0089722 A1
(43) Pub. Date: Mar. 24, 2022

(54) HETERODIMERIC FUSION PROTEIN

(71) Applicant: NANTONG YICHEN BIOPHARMA. CO. LTD., Nantong (CN)

(72) Inventors: Feng WANG, Shanghai (CN); Huayang ZHENG, Shanghai (CN); Yuhan ZHANG, Shanghai (CN)

(21) Appl. No.: 17/419,314

(22) PCT Filed: Dec. 28, 2019
(86) PCT No.: PCT/CN2019/129591

§ 371 Date: Jun. 29, 2021

(30) Foreign Application Priority Data
Dec. 29, 2018 (CN) 201811641942.2

(51) Int. Cl. C07K 16/28; A61P 35/00

(52) U.S. Cl. CPC C07K 16/2803; A61K 2039/505; C07K 16/2863; C07K 16/2833; C07K 16/2878; C07K 16/2851; A61P 35/00; C07K 2317/622; C07K 2319/33; C07K 2319/30; C07K 2317/73; C07K 2317/31; C07K 2317/92; C07K 2317/94; C07K 2317/55; C07K 16/2809

(57) ABSTRACT

Disclosed are a heterodimeric fusion protein and its preparation and application The heavy chain and the light chain of the first antigen binding domain Fab are fused to N-terminus of two Fc chains, respectively, and on this basis, the second antigen binding domain scfv, Fab2, Fv, nanobody or a biologically active peptide is fused to N-terminus of Fab or C-terminus of the Fc. The heterodimeric fusion protein obtained has the following characteristics: each domain in fusion proteins maintained its antigen binding capacity, functional conformation and pharmaceutical properties; at least one antigen binding domain can bind to antigen monovalently; the heterodimeric fusion proteins have a prolonged half-life due to the Fc fragment.

### CROSS-REFERENCES TO RELATED APPLICATION

The present application is a National Stage of International Patent Application No: PCT/CN2019/129591 filed on Dec. 28, 2019, which claims the benefit of the priority of the Chinese patent application with the application No. 201811641942.2, filed to the China National Intellectual Property Administration on Dec. 29, 2018, the entire content of which is incorporated in this application by reference.

### SEQUENCE LISTING

The present application contains a Sequence Listing which has been submitted electronically in ASCII format and is hereby incorporated by reference in its entirety. Said ASCII copy contains 41 new sequences from SEQ ID NO: 152 to SEQ ID NO: 192 described in Specification of this file, but not numbered. The original sequences of SEQ ID NO: 1 to SEQ ID NO: 151 are identical to the sequence listing filed in the corresponding international application No. PCT/CN2019/129591 filed on Dec. 28, 2019.

### TECHNICAL FIELD

The present disclosure belongs to the field of biophamaceuticals, and particularly, relates to a heterodimeric fusion protein based on an immunoglobulin (Ig) fragment, a preparation method therefor, and an application thereof.

### BACKGROUND

Since the first therapeutic antibody muromonab-CD3 used for treating organ grafting-related acute rejection was approved by Food and Drug Administration (FDA) in 1986, there are more than 80 therapeutic monoclonal antibodies approved by FDA, and 2-3 monoclonal antibodies have been approved every year on average up to May 2018. In recent years, the approval of therapeutic monoclonal antibodies has achieved a sustained growth. There were 10 therapeutic monoclonal antibodies approved in 2015, 10 approved in 2016, and in 2017, the number of therapeutic monoclonal antibodies approved by FDA has broke the record, reaching up to 17. It is estimated that market shares of the therapeutic monoclonal antibodies in 2020 will exceed 125 billion dollars (Expert Opin Ther Pat. 2018; 28(3):251-276). Even though we have achieved a great success in the market of monoclonal antibodies, the defects of monoclonal antibody therapy cannot be ignored. Conventional antibodies only bind to a single epitope of a single target and thus, has limited efficacy to some extent. Pharmacological researches reveal that most of the complex diseases are related to various kinds of signal pathways associated with diseases, for example, tumor necrosis factor (TNF), interleukin 6 and other multiple pro inflammatory cytokines; meanwhile, autoimmune/inflammatory diseases are mediated; moreover, the proliferation of tumor cells is always caused by the abnormal up-regulation of multiple growth factor receptors. The blocking of a single signal pathway usually has limited efficacy and easily produces drug tolerance.

Therefore, to develop a bispecific antibody capable of binding two different epitopes from the same or different antigens simultaneously becomes an important topic in the field of research and development of a novel antibody structure for a long time. Up to now, 3 bispecific antibodies have been approved for marketing. The first bispecific antibody is Catumaxomab (Removab®) (anti-EpCAM×anti-CD3) produced by Frenesius and Trion by means of the hybridization and fusion of hybridoma cells from mice and rats; and has been approved by European Medicines Agency (EMA) for treating malignant ascites caused by Epithelial cell adhesion molecule (EpCAM) positive cancer in 2009. In December 2014, the bispecific targeting (anti-CD3×anti-CD19) antibody drug Blinatumomab (Blincyto®) developed by Amgen was approved by FDA for treating Ph-relapsed or refractory acute B cell lymphoblastic leukemia (ALL). Blinatumomab has enjoyed all examination and approval preferential provided by FDA, including examination and approval priority, accelerated approval, accreditation of breakthrough therapies and orphan drugs. In 2017, Emicizumab (Hemlibra®) (anti-factor X×anti-factor IX) of Chugai and Roche was approved by FDA for the treatment of hemophilia A. Statistically, more than 60 bispecific antibodies are in the stage of preclinical study now, and more than 30 bispecific antibodies are in different clinical trial stages. According to Bispecific antibody therapeutics market (3^{rd} edition), 2017-2030) in 2014, it is estimated that up to December 2024, market shares of the bispecific antibody in each year will be up to 5.8 billion dollars; therefore, the bispecific antibody has an enormous market demand.

Based on the molecular structure, the bispecific antibodies which are in the stage of preclinical study and clinical study at present may be divided into two categories: an immunoglobulin-like (IgG-like) bispecific antibody and a non-immunoglobulin-like (non-IgG-like) bispecific antibody based on antibody fragments. The non-IgG-like bispecific antibody represented by BiTE technology platform has a small molecular weight, good tissue penetration, but has very short in vivo half-life for the shortage of Fc fragments. For example, the in vivo half-life of Blincyto is only 2 hi and patients have poor compliance since it requires continuous parenteral administration in clinical practic; in addition, its poor stability easily produces amorphous aggregation. The IgG-like bispecific antibody contains Fc fragments to retain the effector functions, such as, Fc-mediated antibody-dependent cell-mediated cytotoxicity (ADCC), complement-dependent cytotoxicity (CDC), and antibody-dependent cellular phagocytosis (ADCP). Moreover, Fc may be used to purify IgG-like bispecific antibodies, thus facilitating the improvement of solubility and greatly improving the stability of such bispecific antibodies; due to large molecular weight and the FcRn-mediated recirculation, the Ig-like bispecific antibody has a prolonged half-life. The Ig-like bispecific antibody represented by Roche's CrossMab technology attempted to solve the mispairing problems of light chain-light chain pairing, heavy chain-heavy chain pairing, and heavy chain-light chain of the two antibody fragments by the interchange of VH and VL, CH1 and CL or VH-CH1 and VL-CL, and knobs-into-holes on Fc. And now, multiple BsAbs developed by utilizing this technology are in the clinical trial stages. However, the interchange of domains impair the binding and of the non-conventional Fab to some extent, and the pharmacokinetic properties are not optimal. As for the homologous 2+2 bispecific antibody represented by DVD-Ig, both arms of the Fc thereof can individually bind to two antigens to greatly improve the affinity of BsAb; but as a homodimer, the homologous 2+2 bispecific antibody easily “auto”-activates T cells. Thereby, such BsAbs are not applicable to recruit T cells to kill tumor cells. Therefore, the preparation of BsAbs with excellent pharmacokinetic properties, relatively manufactural process, and affording optimal antigen-binding activities is essential for clinical drug development.

### SUMMARY

The present disclosure provides such a heterodimeric fusion protein; where a Fab heavy chain (FabH) and a light chain (FabL) constituting a first antigen binding domain Fab are fused (directly or by a linker) to N-terminal of two single Fc chains, respectively, so that the structure formed has stable folding similar to that of antibody. Based on this, the present disclosure further provides such a heterodimeric fusion protein, where a second antigen binding domain such as scfv, Fab2, Fv, a nanobody or a biologically active peptide is fused to N-terminal of the Fab heavy chain and/or light chain or C-terminal of any one of the Fc chains. The first antigen binding domain and the second antigen binding domain of the heterodimeric fusion protein may individually retain their own functional conformation and pharmaceutical properties. At least one antigen binding domain can bind to an antigen monovalently. Moreover, the heterodimeric fusion protein of the present disclosure has a prolonged half-life due to the Fc fragment.

Based on this, on the one hand, the present disclosure provides a heterodimeric fusion protein based on an Ig fragment; and the heterodimeric fusion protein includes:

a) a first polypeptide chain, including a Fab heavy chain and a first Fc schain, where the Fab heavy chain is fused to the N-terminal of the first single chain Fc directly or via a linker;
b) a second polypeptide chain, including a Fab light chain and a second Fc chain, where the Fab light chain is fused to the N-terminal of the second Fc chain directly or via a linker;
where, the Fab heavy chain of the first polypeptide chain and the Fab light chain of the second polypeptide chain form the first antigen binding domain, and the first and the second Fc chains form an Fc dimerization domain (FIG. 1A).

In some embodiments, the linker is GGSGAKLAALKAKLAALKGGGGS(SEQ ID NO. 184). In some embodiments, the linker is GGGGSELAALEAELAALEAGGSG(SEQ ID NO. 185). In some embodiments, the Fab heavy chain of the first polypeptide chain is fused to N-terminal of the first Fc chain via a linker GGSGAKLAALKAKLAALKGGGGS(SEQ ID NO. 184); and the Fab light chain of the second polypeptide chain is fused to N-terminal of the second Fc chain via a linker GGGGSELAALEAELAALEAGGSG(SEQ ID NO. 185). In some embodiments, the Fab heavy chain of the first polypeptide chain is fused to N-terminal of the first Fc chain via GGGGSELAALEAELAALEAGGSG(SEQ ID NO. 185); and the Fab light chain of the second polypeptide chain is fused to N terminal of the second Fc chain via a linker GGSGAKLAALKAKLAALKGGGGS (SEQ ID NO. 184).

The first Fc chain and the second Fc chain are preferably derived from the same isotype family (e.g. IgG1) or from different isotype families (e.g. IgG1 and IgG3) as long as they can pair to form a dimer. In some embodiments, the first Fc chain and the second Fc chain are derived from IgG, and more specifically, derived from IgG1. The first Fc chain and the second Fc chain can pair into a dimer by an interchain disulfide bond and intermolecular interaction. In some embodiments, the first Fc chain and the second Fc chain are wild-type Fc. In some embodiments, the wild-type Fc has an amino acid sequence as shown in SEQ ID NO. 147. In some embodiments, the first Fc chain and the second Fc chain are Fc variants. In some embodiments, the Fc variant is free of glycosylation. In some embodiments, the Fc variant contains amino acid substitution on N297 glycosylation site. In some embodiments, the Fc variant containing N297 deglycosylaiton modification has an amino acid sequence as shown in SEQ ID NO. 143. In some embodiments, the Fc variant contains one or more amino acids replacement which reduce Fc binding to an Fc receptor and/or effector function thereof. In some embodiments, the amino acid substitutions in the Fc variant contains one or more of E233P, L234V, L235A, delG236, A327G, A330S, and A331S. In some embodiments, the Fc variant containing one or more amino acid substitutions which reduce Fc binding to an Fc receptor and/or effector function thereof has an amino acid sequence as shown in SEQ ID NO. 144. In some embodiments, one of the first Fc variant and the second Fc variant further contains amino acid substitutions S354C and T366W; and the other variant Fc further contains amino acid substitutions Y349C, T366S, L368A and Y407V. In some embodiments, the first Fc variant has an amino acid sequence as shown in SEQ ID NO. 145; and the second variant Fc has an amino acid sequence as shown in SEQ ID NO. 146. In some embodiments, the first Fc variant has an amino acid sequence as shown in SEQ ID NO. 146; and the second Fc variant has an amino acid sequence as shown in SEQ ID NO. 145. In some embodiments, the first Fc variant has an amino acid sequence as shown in SEQ ID NO. 147; and the second Fc variant has an amino acid sequence as shown in SEQ ID NO. 148. In some embodiments, the first Fc variant has an amino acid sequence as shown in SEQ ID NO. 148; and the second Fc variant has an amino acid sequence as shown in SEQ ID NO. 147.

In some embodiments, the heterodimeric fusion protein further contains a second antigen binding domain.

In some embodiments, the second antigen binding domain of the heterodimeric fusion protein is a single chain Fv (scfv).

In some embodiments, scfv constituting the second antigen binding domain is fused to N-terminal of the first polypeptide chain directly or by a linker (FIG. 1B).

In some embodiments, the first antigen binding domain Fab of the heterodimeric fusion protein binds to CD3. In some embodiments, the first antigen binding domain Fab binding to CD3 has a Fab heavy chain as shown in SEQ ID NO. 125 and a Fab light chain as shown in SEQ ID NO. 126. In some embodiments, the second antigen binding domain scfv of the heterodimeric fusion protein binds to EGFR. In some embodiments, the second antigen binding domain scfv binding to EGFR has an amino acid sequence as shown in SEQ ID NO. 142. In some embodiments, the heterodimeric fusion protein binds to CD3 and EGFR. In some embodiments, the first antigen binding domain of the heterodimeric fusion protein binding to CD3 and EGFR has a Fab heavy chain as shown in SEQ ID NO. 125 and a Fab light chain as shown in SEQ ID NO. 126; and the second antigen binding domain thereof has an amino acid sequence as shown in SEQ ID NO. 142. In some embodiments, the heterodimeric fusion protein binding to CD3 and EGFR has a first polypeptide chain containing an amino acid sequence as shown in SEQ ID NO. 18 and a second polypeptide chain containing an amino acid sequence as shown in SEQ ID NO. 12.

In some embodiments, scfv constituting the second antigen binding domain is fused to N-terminal of the second polypeptide chain directly or by a linker (FIG. 1C).

In some embodiments, the first antigen binding domain Fab of the heterodimeric fusion protein binds to CD3. In some embodiments, the first antigen binding domain Fab binding to CD3 has a FabH as shown in SEQ ID NO. 125 and a FabL as shown in SEQ ID NO. 126. In some embodiments, the second antigen binding domain scfv of the heterodimeric fusion protein binds to EGFR. In some embodiments, the second antigen binding domain scfv binding to EGFR has an amino acid sequence as shown in SEQ ID NO. 142. In some embodiments, the heterodimeric fusion protein binds to CD3 and EGFR. In some embodiments, the first antigen binding domain of the heterodimeric fusion protein binding to CD3 and EGFR has a Fab heavy chain as shown in SEQ ID NO. 125 and a Fab light chain as shown in SEQ ID NO. 126; and the second antigen binding domain thereof has an amino acid sequence as shown in SEQ ID NO. 142. In some embodiments, the heterodimeric fusion protein binding to CD3 and EGFR has a first polypeptide chain having an amino acid sequence as shown in SEQ ID NO.30 and a second polypeptide chain having an amino acid sequence as shown in SEQ ID NO.58.

In some embodiments, scfv constituting the second antigen binding domain is fused to C-terminal of the first polypeptide chain directly or by a linker (FIG. 1D).

In some embodiments, the first antigen binding domain Fab of the heterodimeric fusion protein binds to CD3. In some embodiments, the first antigen binding domain Fab binding to CD3 has a Fab heavy chain as shown in SEQ ID NO. 125 and a Fab light chain as shown in SEQ ID NO. 126. In some embodiments, the first antigen binding domain Fab binding to CD3 has a Fab heavy chain as shown in SEQ ID NO. 129 and a Fab light chain as shown in SEQ ID NO. 130. In some embodiments, the first antigen binding domain Fab binding to CD3 has a Fab heavy chain as shown in SEQ ID NO. 131 and a Fab light chain as shown in SEQ ID NO. 132. In some embodiments, scfv constituting the second antigen binding domain of the heterodimeric fusion protein binds to CD19. In some embodiments, scfv constituting the second antigen binding domain binding to CD19 has an amino acid sequence as shown in SEQ ID NO. 139. In some embodiments, scfv constituting the second antigen binding domain of the heterodimeric fusion protein binds to EGFR. In some embodiments, scfv constituting the second antigen binding domain binding to EGFR has an amino acid sequence as shown in SEQ ID NO. 142. In some embodiments, the heterodimeric fusion protein binds to CD3 and CD19. In some embodiments, the first antigen binding domain of the heterodimeric fusion protein binding to CD3 and CD19 has a Fab heavy chain as shown in SEQ ID NO. 125 and a Fab light chain as shown in SEQ ID NO. 126; and the second antigen binding domain thereof has an amino acid sequence as shown in SEQ ID NO. 139. In some embodiments, the heterodimeric fusion protein binding to CD3 and CD19 has a first polypeptide chain having an amino acid sequence as shown in SEQ ID NO.2 and a second polypeptide chain having an amino acid sequence as shown in SEQ ID NO.4. In some embodiments, the first antigen binding domain of the heterodimeric fusion protein binding to CD3 and CD19 has a Fab heavy chain as shown in SEQ ID NO. 129 and a Fab light chain as shown in SEQ ID NO. 130; and the second antigen binding domain thereof has an amino acid sequence as shown in SEQ ID NO. 139. In some embodiments, the heterodimeric fusion protein binding to CD3 and CD19 has a first polypeptide chain having an amino acid sequence as shown in SEQ ID NO. 6 and a second polypeptide chain having an amino acid sequence as shown in SEQ ID NO. 8. In some embodiments, the heterodimeric fusion protein binds to CD3 and EGFR. In some embodiments, the first antigen binding domain of the heterodimeric fusion protein binding to CD3 and EGFR has a Fab heavy chain as shown in SEQ ID NO. 131 and a Fab light chain as shown in SEQ ID NO. 132; and the second antigen binding domain thereof has an amino acid sequence as shown in SEQ ID NO. 142. In some embodiments, the heterodimeric fusion protein binding to CD3 and EGFR has a first polypeptide chain having an amino acid sequence as shown in SEQ ID NO. 14 and a second polypeptide chain having an amino acid sequence as shown in SEQ ID NO. 16.

In some embodiments, scfv constituting the second antigen binding domain is fused to C-terminal of the second polypeptide chain directly or by a linker (FIG. 1E).

In some embodiments, the first antigen binding domain Fab of the heterodimeric fusion protein binds to CD3. In some embodiments, the first antigen binding domain Fab binding to CD3 has a Fab heavy chain as shown in SEQ ID NO. 125 and a Fab light chain as shown in SEQ ID NO. 126. In some embodiments, the first antigen binding domain Fab binding to CD3 has a Fab heavy chain as shown in SEQ ID NO. 129 and a Fab light chain as shown in SEQ ID NO. 130. In some embodiments, the first antigen binding domain Fab binding to CD3 has a Fab heavy chain as shown in SEQ ID NO. 131 and a Fab light chain as shown in SEQ ID NO. 132. In some embodiments, scfv constituting the second antigen binding domain of the heterodimeric fusion protein binds to CD19. In some embodiments, scfv constituting the second antigen binding domain binding to CD19 has an amino acid sequence as shown in SEQ ID NO. 139. In some embodiments, scfv constituting the second antigen binding domain of the heterodimeric fusion protein binds to BCMA. In some embodiments, scfv constituting the second antigen binding domain binding to BCMA has an amino acid sequence as shown in SEQ ID NO. 140. In some embodiments, the second antigen binding domain scfv of the heterodimeric fusion protein binds to CLL-1. In some embodiments, scfv constituting the second antigen binding domain binding to CLL-1 has an amino acid sequence as shown in SEQ ID NO. 141. In some embodiments, scfv constituting the second antigen binding domain of the heterodimeric fusion protein binds to EGFR. In some embodiments, scfv constituting the second antigen binding domain binding to EGFR has an amino acid sequence as shown in SEQ ID NO. 142. In some embodiments, the heterodimeric fusion protein binds to CD3 and CD19. In some embodiments, the first antigen binding domain of the heterodimeric fusion protein binding to CD3 and CD19 has a Fab heavy chain as shown in SEQ ID NO. 125 and a Fab light chain as shown in SEQ ID NO. 126; and the second antigen binding domain thereof has an amino acid sequence as shown in SEQ ID NO. 139. In some embodiments, the first antigen binding domain of the heterodimeric fusion protein binding to CD3 and CD19 has a Fab heavy chain as shown in SEQ ID NO. 129 and a Fab light chain as shown in SEQ ID NO. 130; and the second antigen binding domain thereof has an amino acid sequence as shown in SEQ ID NO. 139. In some embodiments, the heterodimeric fusion protein binding to CD3 and CD19 has a first polypeptide chain having an amino acid sequence as shown in SEQ ID NO. 20 and a second polypeptide chain having an amino acid sequence as shown in SEQ ID NO. 22. In some embodiments, the heterodimeric fusion protein binding to CD3 and CD19 has a first polypeptide chain having an amino acid sequence as shown in SEQ ID NO. 24 and a second polypeptide chain having an amino acid sequence as shown in SEQ ID NO. 26. In some embodiments, the heterodimeric fusion protein binds to CD3 and BCMA. In some embodiments, the first antigen binding domain of the heterodimeric fusion protein binding to CD3 and EGFR has an Fab heavy chain as shown in SEQ ID NO. 125 and an Fab light chain as shown in SEQ ID NO. 126; and the second antigen binding domain thereof has an amino acid sequence as shown in SEQ ID NO. 140. In some embodiments, the heterodimeric fusion protein binding to CD3 and BCMA has a first polypeptide chain having an amino acid sequence as shown in SEQ ID NO.30 and a second polypeptide chain having an amino acid sequence as shown in SEQ ID NO.40. In some embodiments, the heterodimeric fusion protein binds to CD3 and CLL-1. In some embodiments, the first antigen binding domain of the heterodimeric fusion protein binding to CD3 and CLL-1 has a Fab heavy chain as shown in SEQ ID NO. 125 and a Fab light chain as shown in SEQ ID NO. 126; and the second antigen binding domain thereof has an amino acid sequence as shown in SEQ ID NO. 141. In some embodiments, the heterodimeric fusion protein binding to CD3 and CLL-1 has a first polypeptide chain containing an amino acid sequence as shown in SEQ ID NO.30 and a second polypeptide chain containing an amino acid sequence as shown in SEQ ID NO.42. In some embodiments, the heterodimeric fusion protein binds to CD3 and EGFR. In some embodiments, the first antigen binding domain of the heterodimeric fusion protein binding to CD3 and EGFR has a Fab heavy chain as shown in SEQ ID NO. 125 and a Fab light chain as shown in SEQ ID NO. 126; and the second antigen binding domain thereof has an amino acid sequence as shown in SEQ ID NO. 142. In some embodiments, the heterodimeric fusion protein binding to CD3 and EGFR has a first polypeptide chain having an amino acid sequence as shown in SEQ ID NO.24 and a second polypeptide chain having an amino acid sequence as shown in SEQ ID NO.28. In some embodiments, the heterodimeric fusion protein binding to CD3 and EGFR has a first polypeptide chain containing an amino acid sequence as shown in SEQ ID NO.30 and a second polypeptide chain containing an amino acid sequence as shown in SEQ ID NO.32. In some embodiments, the heterodimeric fusion protein binding to CD3 and EGFR has a first polypeptide chain having an amino acid sequence as shown in SEQ ID NO.54 and a second polypeptide chain having an amino acid sequence as shown in SEQ ID NO. 56. In some embodiments, the heterodimeric fusion protein binding to CD3 and EGFR has a first polypeptide chain having an amino acid sequence as shown in SEQ ID NO.44 and a second polypeptide chain having an amino acid sequence as shown in SEQ ID NO.46. In some embodiments, the heterodimeric fusion protein binding to CD3 and EGFR has a first polypeptide chain having an amino acid sequence as shown in SEQ ID NO.44 and a second polypeptide chain having an amino acid sequence as shown in SEQ ID NO. 52. In some embodiments, the heterodimeric fusion protein binding to CD3 and EGFR has a first polypeptide chain having an amino acid sequence as shown in SEQ ID NO.48 and a second polypeptide chain having an amino acid sequence as shown in SEQ ID NO.50. In some embodiments, the first antigen binding domain of the heterodimeric fusion protein binding to CD3 and EGFR has a Fab heavy chain as shown in SEQ ID NO. 131 and a Fab light chain as shown in SEQ ID NO. 132; and the second antigen binding domain thereof has an amino acid sequence as shown in SEQ ID NO. 142. In some embodiments, the heterodimeric fusion protein binding to CD3 and EGFR has a first polypeptide chain having an amino acid sequence as shown in SEQ ID NO.36 and a second polypeptide chain having an amino acid sequence as shown in SEQ ID NO.38.

In some embodiments, the second antigen binding domain of the heterodimeric fusion protein is a biologically active peptide.

In some embodiments, the biologically active peptide is fused to N-terminal of the first polypeptide chain directly or by a linker (FIG. 1B).

In some embodiments, the biologically active peptide is fused to N-terminal of the second polypeptide chain directly or by a linker (FIG. 1C).

In some embodiments, the biologically active peptide is fused to C terminal of the first polypeptide chain directly or by a linker (FIG. 1D).

In some embodiments, the biologically active peptide is EGF4. In some embodiments, the biologically active peptide EGF4 has an amino acid sequence as shown in SEQ ID NO. 150. In some embodiments, the first antigen binding domain Fab of the heterodimeric fusion protein binds to CD3. In some embodiments, the first antigen binding domain Fab binding to CD3 has a Fab heavy chain as shown in SEQ ID NO. 125 and a Fab light chain as shown in SEQ ID NO. 126. In some embodiments, the heterodimeric fusion protein binds to CD3 and EGFR. In some embodiments, the first antigen binding domain of the heterodimeric fusion protein binding to CD3 and EGFR has a Fab heavy chain as shown in SEQ ID NO. 125 and a Fab light chain as shown in SEQ ID NO. 126; and the biologically active peptide of the second antigen binding domain thereof has an amino acid sequence as shown in SEQ ID NO. 150. In some embodiments, the heterodimeric fusion protein binding to CD3 and EGFR has a first polypeptide chain having an amino acid sequence as shown in SEQ ID NO. 10 and a second polypeptide chain having an amino acid sequence as shown in SEQ ID NO. 12.

In some embodiments, the biologically active peptide is fused to C-terminal of the second polypeptide chain directly or a linker (FIG. 1E).

In some embodiments, the biologically active peptide is EGF4. In some embodiments, the biologically active peptide EGF4 has an amino acid sequence as shown in SEQ ID NO. 150. In some embodiments, the first antigen binding domain Fab of the heterodimeric fusion protein binds to CD3. In some embodiments, the first antigen binding domain Fab binding to CD3 has a Fab heavy chain as shown in SEQ ID NO. 125 and a Fab light chain as shown in SEQ ID NO. 126. In some embodiments, the first antigen binding domain of the heterodimeric fusion protein has a Fab heavy chain as shown in SEQ ID NO. 125 and a Fab light chain as shown in SEQ ID NO. 126; and the second antigen binding domain thereof has an amino acid sequence as shown in SEQ ID NO. 150. In some embodiments, the heterodimeric fusion protein has a first polypeptide chain having an amino acid sequence as shown in SEQ ID NO.30 and a second polypeptide chain having an amino acid sequence as shown in SEQ ID NO.34. In some embodiments, the second antigen binding domain of the heterodimeric fusion protein consists of a first biologically active peptide and a second biologically active peptide.

In some embodiments, the first biologically active peptide and the second biologically active peptide are fused to N-terminal of the first polypeptide chain and the second polypeptide chain directly or via a linker, respectively.

In some embodiments, the first biologically active peptide and the second biologically active peptide are fused to C-terminal of the first polypeptide chain and the second polypeptide chain directly or via a linker, respectively (FIG. 1F).

In some embodiments, the first biologically active peptide and the second biologically active peptide are different. In some embodiments, the first biologically active peptide and the second biologically active peptide are the same.

In some embodiments, the first biologically active peptide and the second biologically active peptide are NKG2D. In some embodiments, the biologically active peptide NKG2D has an amino acid sequence as shown in SEQ ID NO.151. In some embodiments, the first antigen binding domain Fab of the heterodimeric fusion protein binds to CD3. In some embodiments, the first antigen binding domain Fab binding to CD3 has a Fab heavy chain as shown in SEQ ID NO. 125 and a Fab light chain as shown in SEQ ID NO. 126. In some embodiments, the heterodimeric fusion protein binds to CD3 and MIC-A. In some embodiments, the first antigen binding domain of the heterodimeric fusion protein binding to CD3 and MIC-A has a Fab heavy chain as shown in SEQ ID NO. 125 and a Fab light chain as shown in SEQ ID NO. 126; and the second antigen binding domain thereof has an amino acid sequence as shown in SEQ ID NO.151. In some embodiments, the heterodimeric fusion protein binding to CD3 and MIC-A has a first polypeptide chain having an amino acid sequence as shown in SEQ ID NO. 122 and a second polypeptide chain having an amino acid sequence as shown in SEQ ID NO. 124.

In some embodiments, the second antigen binding domain of the heterodimeric fusion protein is an Fv.

In some embodiments, the heavy chain variable region of Fv constituting the second antigen binding domain is fused to C-terminal of the first polypeptide chain directly or via a linker; and the light chain variable region of Fv constituting the second antigen binding domain is fused to C-terminal of the second polypeptide chain directly or via a linker (FIG. 1G).

In some embodiments, the first antigen binding domain Fab of the heterodimeric fusion protein binds to CD3. In some embodiments, the first antigen binding domain binding to CD3 has a Fab heavy chain as shown in SEQ ID NO. 125, and a Fab light chain as shown in SEQ ID NO. 126. In some embodiments, Fv constituting the second antigen binding domain of the heterodimeric fusion protein binds to EGFR. In some embodiments, Fv constituting the second antigen binding domain binding to EGFR has a heavy chain variable region as shown in SEQ ID NO. 135 and a light chain variable region as shown in SEQ ID NO. 136. In some embodiments, the heterodimeric fusion protein binds to CD3 and EGFR. In some embodiments, the first antigen binding domain of the heterodimeric fusion protein binding to CD3 and EGFR has a Fab heavy chain as shown in SEQ ID NO. 125 and a Fab light chain as shown in SEQ ID NO. 126; and the second antigen binding domain thereof has a heavy chain variable region as shown in SEQ ID NO. 135 and a light chain variable region as shown in SEQ ID NO. 136. In some embodiments, the heterodimeric fusion protein binding to CD3 and EGFR has a first polypeptide chain containing an amino acid sequence as shown in SEQ ID NO.60 and a second polypeptide chain containing an amino acid sequence as shown in SEQ ID NO.62. In some embodiments, the heterodimeric fusion protein binding to CD3 and EGFR has a first polypeptide chain having an amino acid sequence as shown in SEQ ID NO.64 and a second polypeptide chain having an amino acid sequence as shown in SEQ ID NO.66. In some embodiments, the heterodimeric fusion protein binding to CD3 and EGFR has a first polypeptide chain having an amino acid sequence as shown in SEQ ID NO.68 and a second polypeptide chain having an amino acid sequence as shown in SEQ ID NO.70.

In some embodiments, the heavy chain variable region of the second antigen binding domain Fv is connected to a C terminal end of the second polypeptide chain directly or via a linker; and the light chain variable region of the second antigen binding domain Fv is connected to a C terminal end of the first polypeptide chain directly or via a linker (FIG. 1H).

In some embodiments, the first antigen binding domain Fab of the heterodimeric fusion protein binds to CD3. In some embodiments, the first antigen binding domain binding to CD3 has a Fab heavy chain as shown in SEQ ID NO. 125, and a Fab light chain as shown in SEQ ID NO. 126. In some embodiments, the second antigen binding domain of the heterodimeric fusion protein binds to EGFR. In some embodiments, Fv constituting the second antigen binding domain binding to EGFR has a heavy chain variable region as shown in SEQ ID NO. 135 and a light chain variable region as shown in SEQ ID NO. 136. In some embodiments, the heterodimeric fusion protein binds to CD3 and EGFR. In some embodiments, the first antigen binding domain of the heterodimeric fusion protein binding to CD3 and EGFR has a Fab heavy chain as shown in SEQ ID NO. 125 and a Fab light chain as shown in SEQ ID NO. 126; and the second antigen binding domain thereof has a heavy chain variable region as shown in SEQ ID NO. 135 and a light chain variable region as shown in SEQ ID NO. 136. In some embodiments, the heterodimeric fusion protein binding to CD3 and EGFR has a first polypeptide chain having an amino acid sequence as shown in SEQ ID NO.72, and a second polypeptide chain having an amino acid sequence as shown in SEQ ID NO.74. In some embodiments, the heterodimeric fusion protein binding to CD3 and EGFR has a first polypeptide chain having an amino acid sequence as shown in SEQ ID NO.76 and a second polypeptide chain having an amino acid sequence as shown in SEQ ID NO.78. In some embodiments, the heterodimeric fusion protein binding to CD3 and EGFR has a first polypeptide chain having an amino acid sequence as shown in SEQ ID NO.80 and a second polypeptide chain having an amino acid sequence as shown in SEQ ID NO. 82.

In some embodiments, the heavy chain variable region of Fv constituting the second antigen binding domain is fused to N-terminal of the second polypeptide chain directly or via a linker; and the light chain variable region of Fv constituting the second antigen binding domain is fused to N-terminal of the first polypeptide chain directly or via a linker (FIG. 1J).

In some embodiments, the heavy chain variable region of Fv constituting the second antigen binding domain is fused to N-terminal of the first polypeptide chain directly or via a linker; and the light chain variable region of the second antigen binding domain Fv is fused to N-terminal of the second polypeptide chain directly or via a linker (FIG. 1I).

In some embodiments, the first antigen binding domain of the heterodimeric fusion protein binds to EGFR. In some embodiments, the first antigen binding domain binding to EGFR has a Fab heavy chain as shown in SEQ ID NO. 127, and a Fab light chain as shown in SEQ ID NO. 128. In some embodiments, the second antigen binding domain of the heterodimeric fusion protein binds CD3. In some embodiments, Fv constituting the second antigen binding domain binding to CD3 has a heavy chain variable region as shown in SEQ ID NO. 133 and a light chain variable region as shown in SEQ ID NO. 134. In some embodiments, the heterodimeric fusion protein binds to CD3 and EGFR. In some embodiments, the first antigen binding domain of the heterodimeric fusion protein binding to CD3 and EGFR has a Fab heavy chain as shown in SEQ ID NO. 127 and a Fab light chain as shown in SEQ ID NO. 128; and the second antigen binding domain thereof has a heavy chain variable region as shown in SEQ ID NO. 133 and a light chain variable region as shown in SEQ ID NO. 134. In some embodiments, the heterodimeric fusion protein binding to CD3 and EGFR has a first polypeptide chain having an amino acid sequence as shown in SEQ ID NO. 104 and a second polypeptide chain having an amino acid sequence as shown in SEQ ID NO. 106.

In some embodiments, the first antigen binding domain Fab of the heterodimeric fusion protein binds to CD3. In some embodiments, the first antigen binding domain Fab binding to CD3 has a Fab heavy chain as shown in SEQ ID NO. 125 and a Fab light chain as shown in SEQ ID NO. 126. In some embodiments, Fv constituting the second antigen binding domain of the heterodimeric fusion protein binds to EGFR. In some embodiments, the second antigen binding domain binding to EGFR has a heavy chain variable region as shown in SEQ ID NO. 135 and a light chain variable region as shown in SEQ ID NO. 136. In some embodiments, the heterodimeric fusion protein binds to CD3 and EGFR. In some embodiments, the first antigen binding domain of the heterodimer binding to CD3 and EGFR has a Fab heavy chain as shown in SEQ ID NO. 125 and a Fab light chain as shown in SEQ ID NO. 126; and the second antigen binding domain Fv thereof has a heavy chain variable region as shown in SEQ ID NO. 135 and a light chain variable region as shown in SEQ ID NO. 136. In some embodiments, the heterodimeric fusion protein binding to CD3 and EGFR has a first polypeptide chain having an amino acid sequence as shown in SEQ ID NO. 108 and a second polypeptide chain having an amino acid sequence as shown in SEQ ID NO.110.

In some embodiments, the second antigen binding domain of the heterodimeric fusion protein is Fab2.

In some embodiments, the heavy chain of the second antigen binding domain Fab2 is fused to C-terminal of the first polypeptide chain directly or via a linker; and the light chain of the Fab2 is fused to C-terminal of the second polypeptide chain directly or via a linker (FIG. 1K).

In some embodiments, the first antigen binding domain Fab of the heterodimeric fusion protein binds to CD3. In some embodiments, the first antigen binding domain Fab binding to CD3 has a Fab heavy chain as shown in SEQ ID NO. 125 and a Fab light chain as shown in SEQ ID NO. 126. In some embodiments, Fab2 constituting the second antigen binding domain of the heterodimer binds to EGFR. In some embodiments, Fab2 constituting the second antigen binding domain binding to EGFR has a Fab heavy chain variable region as shown in SEQ ID NO. 127 and a Fab light chain variable region as shown in SEQ ID NO. 128. In some embodiments, the heterodimeric fusion protein binds to CD3 and EGFR. In some embodiments, the first antigen binding domain Fab of the heterodimeric fusion protein binding to CD3 and EGFR has a Fab heavy chain as shown in SEQ ID NO. 125 and a Fab light chain as shown in SEQ ID NO. 126; and the second antigen binding domain Fab2 thereof has a Fab heavy chain as shown in SEQ ID NO. 127 and a Fab light chain as shown in SEQ ID NO. 128. In some embodiments, the heterodimeric fusion protein binding to CD3 and EGFR has a first polypeptide chain having an amino acid sequence as shown in SEQ ID NO.84 and a second polypeptide chain having an amino acid sequence as shown in SEQ ID NO.86.

In some embodiments, the heavy chain of the second antigen binding domain Fab2 is fused to C-terminal of the second polypeptide chain directly or via a linker; and the light chain of the Fab2 is fused to C-terminal of the first polypeptide chain directly or via a linker (FIG. 1N).

In some embodiments, the first antigen binding domain of the heterodimeric fusion protein binds to CD3. In some embodiments, the first antigen binding domain Fab binding to CD3 has a Fab heavy chain as shown in SEQ ID NO. 125 and a Fab light chain as shown in SEQ ID NO. 126. In some embodiments, the second antigen binding domain Fab2 of the heterodimer binds to EGFR. In some embodiments, the second antigen binding domain Fab2 binding to EGFR ha an Fab heavy chain as shown in SEQ ID NO. 127 and a Fab light chain as shown in SEQ ID NO. 128. In some embodiments, the heterodimeric fusion protein binds to CD3 and EGFR. In some embodiments, the first antigen binding domain Fab of the heterodimeric fusion protein binding to CD3 and EGFR has a Fab heavy chain as shown in SEQ ID NO. 125 and a Fab light chain as shown in SEQ ID NO. 126; and the second antigen binding domain Fab2 thereof has a Fab heavy chain as shown in SEQ ID NO. 127 and a Fab light chain as shown in SEQ ID NO. 128. In some embodiments, the heterodimeric fusion protein binding to CD3 and EGFR has a first polypeptide chain having an amino acid sequence as shown in SEQ ID NO.88 and a second polypeptide chain having an amino acid sequence as shown in SEQ ID NO.90.

In some embodiments, the heavy chain of the second antigen binding domain Fab2 is fused to N-terminal of the second polypeptide chain directly or via a linker; and the light chain of the Fab2 is fused to N-terminal of the first polypeptide chain directly or via a linker (FIG. 1M).

In some embodiments, the heavy chain of the second antigen binding domain Fab2 is fused to N-terminal of the first polypeptide chain directly or via a linker; and the light chain of the Fab2 is fused to N-terminal of the second polypeptide chain directly or via a linker (FIG. 1L).

In some embodiments, the first antigen binding domain Fab of the heterodimeric fusion protein binds to EGFR. In some embodiments, the first antigen binding domain Fab binding to EGFR has a Fab heavy chain as shown in SEQ ID NO. 127 and a Fab light chain as shown in SEQ ID NO. 128. In some embodiments, the second antigen binding domain Fab2 of the heterodimeric fusion protein binds CD3. In some embodiments, the second antigen binding domain Fab2 binding to CD3 has a Fab heavy chain as shown in SEQ ID NO. 125 and a Fab light chain as shown in SEQ ID NO. 126. In some embodiments, the heterodimer binds to EGFR and CD3. In some embodiments, the first antigen binding domain Fab of the heterodimeric fusion protein binding to EGFR and CD3 has a Fab heavy chain as shown in SEQ ID NO. 127 and a Fab light chain as shown in SEQ ID NO. 128; and the second antigen binding domain Fab2 thereof has a Fab heavy chain as shown in SEQ ID NO. 125 and a Fab light chain as shown in SEQ ID NO. 126. In some embodiments, the heterodimeric fusion protein binding to EGFR and CD3 has a first polypeptide chain having an amino acid sequence as shown in SEQ ID NO. 112 and a second polypeptide chain having an amino acid sequence as shown in SEQ ID NO. 114.

In some embodiments, the first antigen binding domain Fab of the heterodimeric fusion protein binds to CD3. In some embodiments, the first antigen binding domain binding to CD3 has a Fab heavy chain as shown in SEQ ID NO. 125, and a Fab light chain as shown in SEQ ID NO. 126. In some embodiments, the second antigen binding domain Fab2 of the heterodimeric fusion protein binds to EGFR. In some embodiments, the second antigen binding domain Fab2 binding to EGFR has a Fab heavy chain as shown in SEQ ID NO. 127 and a Fab light chain as shown in SEQ ID NO. 128. In some embodiments, the heterodimeric fusion protein binds to CD3 and EGFR. In some embodiments, the first antigen binding domain Fab of the heterodimeric fusion protein binding to CD3 and EGFR has a Fab heavy chain as shown in SEQ ID NO. 125 and a Fab light chain as shown in SEQ ID NO. 126; and the second antigen binding domain Fab2 thereof has a Fab heavy chain as shown in SEQ ID NO. 127 and a Fab light chain as shown in SEQ ID NO. 128. In some embodiments, the heterodimeric fusion protein binding to CD3 and EGFR has a first polypeptide chain having an amino acid sequence as shown in SEQ ID NO. 116 and a second polypeptide chain having an amino acid sequence as shown in SEQ ID NO. 118.

In some embodiments, the second antigen binding domain of the heterodimeric fusion protein is a nanobody.

In some embodiments, the second antigen binding domain, nanobody, is fused to N-terminal of the first polypeptide chain directly or a linker. In some embodiments, the second antigen binding domain, nanobody, is fused to N-terminal of the second polypeptide chain directly or via a linker.

In some embodiments, the second antigen binding domain, nanobody, is fused to C-terminal of the first polypeptide chain directly or via a linker. In some embodiments, the second antigen binding domain, nanobody, is fused to C-terminal of the second polypeptide chain directly or via a linker.

On the other hand, the present disclosure provides a heterodimeric fusion protein based on an Ig fragment; and the heterodimer includes:

a) a first polypeptide chain, including: Fc, (L1)n, CH1, L2, and VH from N-terminal to C-terminal, sequentially;
b) a second polypeptide chain, including: Fc, (L3)n, CL, L4, and VL from N-terminal to C-terminal, sequentially;
where, n is 0 or 1; L1, L2, L3 and L4 are linkers; VH and VL form a first antigen binding domain Fv.

In some embodiments, the heterodimeric fusion protein further includes a second antigen binding domain.

In some embodiments, the second antigen binding domain is a biologically active peptide.

In some embodiments, the biologically active peptide is fused to N-terminal of the first polypeptide chain or the second polypeptide chain directly or via a linker (FIGS. 1P and 1S).

In some embodiments, the biologically active peptide is fused to C-terminal of the first polypeptide chain or the second polypeptide chain directly or via a linker (FIGS. 1T and 1U).

In some embodiments, the second antigen binding domain is Fab2.

In some embodiments, the heavy chain of the second antigen binding domain Fab2 is fused to N-terminal of the first polypeptide chain directly or via a linker; and the light chain of the Fab2 is fused to N-terminal of the second polypeptide chain directly or via a linker.

In some embodiments, the heavy chain of the second antigen binding domain Fab2 is fused to N-terminal of the second polypeptide chain directly or via a linker; and the light chain of the Fab2 is fused to N-terminal of the first polypeptide chain directly or via a linker.

In some embodiments, the second antigen binding domain of the heterodimeric fusion protein is an Fv.

In some embodiments, the heavy chain variable region of the second antigen binding domain Fv is fused to N-terminal of the first polypeptide chain directly or via a linker; and the light chain variable region of the second antigen binding domain Fv is fused to N-terminal of the second polypeptide chain directly or via a linker.

In some embodiments, the heavy chain variable region of the second antigen binding domain Fv is fused to N-terminal of the second polypeptide chain directly or via a linker; and the light chain variable region of the second antigen binding domain Fv is fused to N-terminal of the first polypeptide chain directly or via a linker.

In some embodiments, the second antigen binding domain is a nanobody.

In some embodiments, the second antigen binding domain nanobody, is fused to N-terminal of the first polypeptide chain directly or via a linker. In some embodiments, the second antigen binding domain nanobody, is fused to N-terminal of the second polypeptide chain directly or via a linker.

In some embodiments, the second antigen binding domain is scfv. In some embodiments, the second antigen binding domain scfv is fused to C-terminal of the first polypeptide chain or the second polypeptide chain directly or via a linker (FIGS. 1T and 1U).

In some embodiments, the second antigen binding domain scfv is fused to N-terminal of the second polypeptide chain directly or via a linker (FIG. 1S).

In some embodiments, the second antigen binding domain scfv is fused to N-terminal of the first polypeptide chain directly or via a linker (FIG. 1P).

In some embodiments, the first antigen binding domain Fv of the heterodimer binds to CD3. In some embodiments, the first antigen binding domain Fv binding to CD3 has a VH as shown in SEQ ID NO. 133 and a VL as shown in SEQ ID NO. 134. In some embodiments, the first antigen binding domain Fv binding to CD3 has a VH as shown in SEQ ID NO. 137 and a VL as shown in SEQ ID NO. 138. In some embodiments, the second antigen binding domain scfv binds to CD19. In some embodiments, the second antigen binding domain scfv binding to CD19 has an amino acid sequence as shown in SEQ ID NO. 139. In some embodiments, the heterodimeric fusion protein binds to CD3 and CD19. In some embodiments, the first antigen binding domain Fv of the heterodimeric fusion protein binding to CD3 and CD19 has a VH as shown in SEQ ID NO. 133 and a VL as shown in SEQ ID NO. 134; and the second antigen binding domain scfv thereof has an amino acid sequence as shown in SEQ ID NO. 139. In some embodiments, the heterodimeric fusion protein binding to CD3 and EGFR has a first polypeptide chain having an amino acid sequence as shown in SEQ ID NO.96 and a second polypeptide chain having an amino acid sequence as shown in SEQ ID NO.98. In some embodiments, the first antigen binding domain Fv of the heterodimeric fusion protein binding to CD3 and CD19 has a VH as shown in SEQ ID NO. 137 and a VL as shown in SEQ ID NO. 138; and the second antigen binding domain scfv thereof has an amino acid sequence as shown in SEQ ID NO. 139. In some embodiments, the heterodimeric fusion protein binding to CD3 and EGFR has a first polypeptide chain having an amino acid sequence as shown in SEQ ID NO.92 and a second polypeptide chain having an amino acid sequence as shown in SEQ ID NO.94.

On the one hand, the present disclosure provides a heterodimeric fusion protein based on an Ig fragment; and the heterodimer includes:

a) a first polypeptide chain, including: Fc, (L1)n, CL, L2, and VH from N-terminal to C-terminal sequentially;
b) a second polypeptide chain, including: Fc, (L3)n, CH1, L2, and VL from N-terminal to C-terminal, sequentially;
where, n is 0 or 1; L1, L2, L3 and L4 are linkers; VH and VL form a first antigen binding domain Fv.

In some embodiments, the heterodimeric fusion protein further includes a second antigen binding domain.

In some embodiments, the second antigen binding domain is a biologically active peptide.

In some embodiments, the biologically active peptide is fused to N-terminal of the first polypeptide chain or second polypeptide chain directly or via a linker (FIGS. 1Q and 1R).

In some embodiments, the biologically active peptide is fused to C-terminal of the first polypeptide chain or the second polypeptide chain directly or via a linker (FIGS. 1V and 1W).

In some embodiments, the second antigen binding domain is an Fab2.

In some embodiments, the heavy chain of the second antigen binding domain Fab2 is fused to N-terminal of the first polypeptide chain directly or via a linker; and the light chain of the Fab2 is fused to N-terminal end of the second polypeptide chain directly or via a linker.

In some embodiments, the heavy chain of the second antigen binding domain Fab2 is fused to N-terminal of the second polypeptide chain directly or via a linker; and the light chain of the Fab2 is fused to N-terminal of the first polypeptide chain directly or via a linker.

In some embodiments, the second antigen binding domain of the heterodimeric fusion protein is an Fv.

In some embodiments, the heavy chain variable region of the second antigen binding domain Fv is fused to N-terminal of the first polypeptide chain directly or via a linker; and the light chain variable region of the second antigen binding domain Fv is fused to N-terminal of the second polypeptide chain directly or via a linker.

In some embodiments, the heavy chain variable region of the second antigen binding domain Fv is fused to N-terminal of the second polypeptide chain directly or via a linker; and the light chain variable region of the second antigen binding domain Fv is fused to N-terminal of the first polypeptide chain directly or via a linker.

In some embodiments, the second antigen binding domain is a nanobody.

In some embodiments, the second antigen binding domain nanobody, is fused to N-terminal of the first polypeptide chain directly or via a linker. In some embodiments, the second antigen binding domain nanobody, is fused to N-terminal of the second polypeptide chain directly or via a linker.

In some embodiments, the second antigen binding domain is scfv. In some embodiments, the second antigen binding domain scfv is fused to C-terminal of the first polypeptide chain or the second polypeptide chain directly or via a linker (FIGS. 1V and 1W).

In some embodiments, the second antigen binding domain scfv is fused to N-terminal of the second polypeptide chain directly or via a linker (FIG. 1Q).

In some embodiments, the second antigen binding domain scfv is fused to N-terminal of the first polypeptide chain directly or via a linker (FIG. 1R).

In some embodiments, the first antigen binding domain of the heterodimer binds to CD3. In some embodiments, the first antigen binding domain Fv binding to CD3 has a VH as shown in SEQ ID NO. 133 and a VL as shown in SEQ ID NO. 134. In some embodiments, the second antigen binding domain scfv binds to CD19. In some embodiments, the second antigen binding domain scfv binding to CD19 has an amino acid sequence as shown in SEQ ID NO. 139. In some embodiments, the heterodimeric fusion protein binds to CD3 CD19. In some embodiments, the first antigen binding domain Fv of the heterodimeric fusion protein binding to CD3 and CD19 has a VH as shown in SEQ ID NO. 133 and a VL as shown in SEQ ID NO. 134; and the second antigen binding domain scfv thereof has an amino acid sequence as shown in SEQ ID NO. 139. In some embodiments, the heterodimeric fusion protein binding to CD3 and CD19 has a first polypeptide chain having an amino acid sequence as shown in SEQ ID NO. 100 and a second polypeptide chain having an amino acid sequence as shown in SEQ ID NO. 102.

In the present disclosure, the heavy chain and a light chain of the first antigen binding domain Fab are fused (directly or via a linker) to N-terminal of each Fc, respectively and on this basis, the second antigen binding domain scfv, Fab2, Fv, nanobody or a biologically active peptide is fused to N-terminal of the Fab or C-terminal of the Fc, which effectively solves the mispairing problems of the heavy chain-heavy chain, light chain-light chain, and heavy chain-light chain between two antigen binding domains.

The heterodimeric fusion protein provided by the present disclosure includes at least one linker. In some embodiments, the linker is optionally selected from the followings: GGGGSGGGGSGGGGS(SEQ ID NO. 156), SGGGGSGGGGSGGGGS(SEQ ID NO. 157), GGSGGSGGGGSGGGG(SEQ ID NO. 187), GGSGGSGGGGSGGGGS(SEQ ID NO. 188), GGSGAKLAALKAKLAALKGGGGS(SEQ ID NO. 184), GGGGSELAALEAELAALEAGGSG(SEQ ID NO. 185), APATSLQSGQLGFQCGELCSASA(SEQ ID NO. 189), ASTKGP(SEQ ID NO.180), TVAAPSVFIFPP(SEQ ID NO. 172), PNLLGGP(SEQ ID NO. 190), GGGGS(SEQ ID NO. 152), and GGGEAAAKEAAAKEAAAKAGG(SEQ ID NO. 197). In some embodiments, the linker is the same. In some embodiments, the linker is different.

On one hand, the present disclosure further provides a polynucleotide encoding the heterodimeric fusion protein of the present disclosure.

On one hand, the present disclosure further relates to an expression vector, including the polynucleotide of the present disclosure.

On one hand, the present disclosure further relates to a host cell, including the expression vector of the present disclosure.

On one hand, the present disclosure further relates to a pharmaceutical composition, including the heterodimeric fusion protein of the present disclosure.

On one hand, the present disclosure further relates to a method for treating a cancer and an autoimmune disease of a subject in need thereof. In some embodiments, the method includes a step of administering an effective amount of the heterodimeric fusion protein provided herein, or a pharmaceutical composition of the heterodimeric fusion protein provided herein and a pharmaceutically acceptable carrier to a subject.

In some embodiments, the present disclosure further provides a method for treating B-cell leukemia of a subject in need thereof; and the method includes a step of administering the heterodimeric fusion protein provided herein to the subject, where the heterodimeric fusion protein may bind to CD3 and CD19. In some embodiments, the heterodimeric fusion protein contains an amino acid sequence selected from: SEQ ID No:2 and SEQ ID No:4; SEQ ID No:6 and SEQ ID No:8; SEQ ID No:20 and SEQ ID No:22; SEQ ID No:24 and SEQ ID No:26; SEQ ID No:92 and SEQ ID No:94; SEQ ID No:96 and SEQ ID No:98; SEQ ID No: 100 and SEQ ID No: 102. In some embodiments, the B-cell leukemia is selected from: Hodgkin's lymphoma, non-Hodgkin lymphoma (NHL), precursor B-cell lymphoblastic leukemia/lymphoma, mature B cell sarcoma, B-cell chronic lymphocytic leukemia/small lymphocytic lymphoma, B-cell prolymphocytic leukemia, lymphoplasmacytoid lymphoma, mantle cell lymphoma, follicular lymphoma, cutaneous follicular center lymphoma, marginal zone lymphomas, hairy cell leukemia, diffuse large B cell lymphoma, Burkitt's lymphoma, plasmacytoma, plasma-cell myeloma, posttransplant lymphoproliferative disorders, Waldenstrom's macroglobulinemia and anaplastic large cell lymphoma.

In some embodiments, the present disclosure further relates to a method for treating a cancer of a subject in need thereof; and in some embodiments, the cancer is selected from a group consisting of the following: melanoma (for example, metastatic malignant melanoma), renal carcinoma (for example, clear cell carcinoma), prostatic cancer (for example, hormone-refractory prostate cancer), pancreatic cancer, mastocarcinoma, colon cancer, lung cancer (for example, non-small cell lung cancer), esophagus cancer, squamous cell carcinoma of the head and neck, liver cancer, ovarian cancer, cervical cancer, thyroid cancer, glioblastoma, glioma, leukemia, lymphoma and other neoplastic malignant diseases.

In some embodiments, the present disclosure further relates to a method for treating EGFR highly-expressed lung cancer and colon cancer of a subject in need thereof; and the method includes a step of administering the heterodimeric fusion protein provided herein to the subject, where the heterodimeric fusion protein may bind to CD3 and EGFR. In some embodiments, the heterodimeric fusion protein contains the following amino acid sequences as shown in SEQ ID No: 10 and SEQ ID No: 12; SEQ ID No: 14 and SEQ ID No: 16; SEQ ID No: 18 and SEQ ID No: 12; SEQ ID No:24 and SEQ ID No:28; SEQ ID No:30 and SEQ ID No:32; SEQ ID No:30 and SEQ ID No:34; SEQ ID No:36 and SEQ ID No:38; SEQ ID No:44 and SEQ ID No:46; SEQ ID No:48 and SEQ ID No:50; SEQ ID No:44 and SEQ ID No:52; SEQ ID No:54 and SEQ ID No:56; SEQ ID No:30 and SEQ ID No:58; SEQ ID No:60 and SEQ ID No:62; SEQ ID No:64 and SEQ ID No:66; SEQ ID No:68 and SEQ ID No:70; SEQ ID No:72 SEQ ID No:74; SEQ ID No:76 and SEQ ID No:78; SEQ ID No:80 and SEQ ID No:82; SEQ ID No:84 and SEQ ID No:86; SEQ ID No:88 and SEQ ID No:90; SEQ ID No: 104 and SEQ ID No: 106; SEQ ID No:108 and SEQ ID No:110; SEQ ID No:112 and SEQ ID No:114; SEQ ID No:116 and SEQ ID No: 118.

In some embodiments, the present disclosure provides a method for treating multiple myeloma of a subject in need thereof; and the method includes a step of administering the heterodimeric fusion protein provided herein to the subject, where the heterodimeric fusion protein may bind to CD3 and BCMA. In some embodiments, the heterodimeric fusion protein has amino acid sequences as shown in SEQ ID NO.30 and SEQ ID No:40.

In some embodiments, the present disclosure provides a method for treating acute myelogenous leukemia of a subject in need thereof; and the method includes a step of administering the heterodimeric fusion protein provided herein to the subject, where the heterodimeric fusion protein may bind to CD3 and CLL-1. In some embodiments, the heterodimeric fusion protein has amino acid sequences as shown in SEQ ID NO.30 and SEQ ID No:42.

In some embodiments, the present disclosure provides a method for treating virus infection of a subject in need thereof; and the method includes a step of administering the heterodimeric fusion protein provided herein to the subject, where the heterodimeric fusion protein may bind to CD3 and MICA. In some embodiments, the heterodimeric fusion protein has amino acid sequences as shown in SEQ ID NO. 122 and SEQ ID No: 124.

### BRIEF DESCRIPTION OF THE DRAWINGS

FIG. 1A is a schematic diagram showing the basic framework of the heterodimeric fusion protein, where the chain comprising the FabH is the first polypeptide chain, and the chain comprising the FabL is the second polypeptide chain. FIG. 1B-FIG. 1F show a schematic diagram of a heterodimer where the first antigen binding domain is Fab, and the second antigen binding domain is scfv or a biologically active peptide. FIG. 1B shows a heterodimeric fusion protein in which the second antigen binding domain is fused to N-terminal of the first polypeptide chain; FIG. 1C shows a heterodimeric fusion protein where the second antigen binding domain is fused to N-terminal of the second polypeptide chain; FIG. 1D shows a heterodimeric fusion protein where the second antigen binding domain is fused to C-terminal of the first polypeptide chain; FIG. 1E shows a heterodimeric fusion protein where the second antigen binding domain is fused to C-terminal of the second polypeptide chain; and FIG. 1F shows a heterodimeric fusion protein where the second antigen binding domain is fused to C-terminal of the first polypeptide chain and the second polypeptide chain, respectively. FIG. 1G-FIG. 1J show schematic diagrams of heterodimeric fusion proteins where the first antigen binding domain is a Fab, and the second antigen binding domain is a Fv. FIG. 1G shows a heterodimeric fusion protein where VH and VL of the second antigen binding domain Fv are fused to C-terminal of the first polypeptide chain and second polypeptide chain, respectively; FIG. 1H shows a heterodimeric fusion protein where VL and VH of the second antigen binding domain Fv are fused to C-terminal of the first polypeptide chain and second polypeptide chain, respectively; FIG. 1I shows a heterodimeric fusion protein where VH and VL of the second antigen binding domain Fv are fused to N-terminal of the first polypeptide chain and second polypeptide chain, respectively; and FIG. 1J shows a heterodimeric fusion protein where VL and VH of the second antigen binding domain Fv are fused to N-terminal of the first polypeptide chain and second polypeptide chain, respectively.

FIG. 1K-FIG. 1N show schematic diagrams of heterodimeric fusion proteins where the first antigen binding domain is a Fab, and the second antigen binding domain is a Fab2. FIG. 1K shows a heterodimeric fusion protein where Fab2H and Fab2L of the second antigen binding domain Fab2 are fused to C-terminal of the first polypeptide chain and second polypeptide chain, respectively; FIG. 1L shows a heterodimeric fusion protein where Fab2L and Fab2H of the second antigen binding domain Fab2 are to C-terminal of the first polypeptide chain and second polypeptide chain, respectively; FIG. 1M shows a heterodimeric fusion protein where Fab2H and Fab2L of the second antigen binding domain Fab2 are fused to N-terminal of the first polypeptide chain and second polypeptide chain, respectively; and FIG. 1N shows a heterodimeric fusion protein where Fab2L and Fab2H of the second antigen binding domain Fab2 are fused to N-terminal of the first polypeptide chain and second polypeptide chains, respectively. FIG. 1O is a schematic diagram showing that VH and VL of Fab in the first antigen binding domain and second antigen binding domain are interchanged on two polypeptide chains. FIG. 1P-FIG. 1X show schematic diagrams of heterodimeric fusion proteins where the first antigen binding domain is Fv, and the second antigen binding domain is scfv or a biologically active peptide. FIG. 1P and FIG. 1Q show heterodimeric fusion proteins where the scfv or biologically active peptide of the second antigen binding domain is fused to N-terminal of the first polypeptide chain; FIG. 1R and FIG. 1S show heterodimeric fusion proteins where the scfv or biologically active peptide of the second antigen binding domain is fused to N terminal of the second polypeptide chain; FIG. 1T and FIG. 1U show heterodimeric fusion proteins where the scfv or biologically active peptide of the second antigen binding domain is fused to C-terminal of the first polypeptide chain; FIG. 1V and FIG. 1W show heterodimeric fusion proteins where the scfv or biologically active peptide of the second antigen binding domain is fused to C-terminal of the second polypeptide chain; and FIG. 1X shows a heterodimeric fusion protein where the biologically active peptide is fused to N-terminal of the first polypeptide chain and second polypeptide chain, respectively.

FIG. 2 shows SDS-PAGE of exemplary heterodimeric fusion proteins purified by Protein A or CH1 resin. “M” denotes the protein marker; denotes sample loading without beta-mercaptothanol; “+” denotes sample loading with beta-mercaptothanol; 1:IgFD-24; 2:IgFD-11; 3:IgFD-25; 4:IgFD-26; 5:IgFD-31; 6:IgFD-27; 7:IgFD-30; 7(CH1): IgFD-30(purified by CH1 resin); 8:IgFD-29; 8(CH1):IgFD-29(purified by CH1 resin); 9:IgFD-28; 9(CH1): IgFD-28(purified by CH1 resin).

FIG. 3-1 shows size exclusion chromatogram of the exemplary heterodimeric fusion proteins. FIG. 3-2 shows ion exchange chromatogram of the exemplary heterodimeric antibodies.

FIG. 4 shows that the binding of anti-CD3/CD19 heterodimeric fusion protein IgFD-6/IgFD-7 at different concentrations to NALM-6 cells as detected by flow cytometry(FCM).

FIG. 5 shows the killing effects of PBMCs on Nalm-6 cells mediated by anti-CD3/CD19 heterodimeric fusion protein IgFD-6/IgFD-7 as detected by flow cytometry.

FIG. 6 shows the binding of different anti-CD3/anti-EGFR heterodimeric fusion proteins to human EGFR as detected by ELISA.

FIG. 7 shows the binding of different anti-CD3/anti-EGFR heterodimeric fusion proteins to F98-EGFR cells as detected by flow cytometry. ProA denotes a fusion protein purified by protein A Resin; and CH1 denotes a fusion protein purified by CH1 resin.

FIG. 8 shows the binding of anti-CD3/anti-EGFR heterodimeric fusion proteins IgFD-8/IgFD-18 and IgFD-19 to PBMC-T cells as detected by flow cytometry.

FIG. 9 shows the binding of different anti-CD3/anti-EGFR heterodimeric fusion proteins to Jurkat T cells; wherein, “ProA” denotes a fusion protein purified by protein A Resin; and “CH1” denotes a fusion protein purified by CH1 resin.

FIG. 10 shows the killing effect of PMBCs on F98-EGFR cells mediated by different anti-CD3/anti-EGFR heterodimeric fusion proteins. “Size” denotes the protein was purified by gel chromatography, and “Monos” denotes the protein was purified by ion exchange chromatography.

FIG. 11 shows the killing effect of PBMCs on MM1.R cells mediated by anti-CD3/BCMA heterodimeric fusion protein IgFD-22 as detected by LDH assay.

FIG. 12 shows the binding of the anti-CD3/MICA heterodimeric fusion protein IgFD-37 to human MICA(IgFD-36 serves as a control) as detected by ELISA.

FIG. 13 shows the binding of the anti-CD3/MICA heterodimeric fusion protein IgFD37 to MICA positive cells; (A) PANC-1 cells; (B) BXPC-3 cells; and (C) K562 cells.

FIG. 14 shows the killing effect of PBMCs on K562 cells (A) and PANC-1 cells (B) medidated by the anti-CD3/MICA heterodimeric fusion protein IgFD-37 as detected by LDH assay.

FIG. 15 shows the killing effect of PMBCs on HL-60 cells mediated by the anti-CD3/CLL-1 heterodimeric fusion protein IgFD-23 as detected by flow cytometry.

FIG. 16 shows the pharmacokinetic analysis of IgFD-25 and IgFD-33 in rats after intraperitoneal administration.

FIG. 17 shows the tumor growth inhibition of IgFD-33 in an A431 lung cancer mouse model.

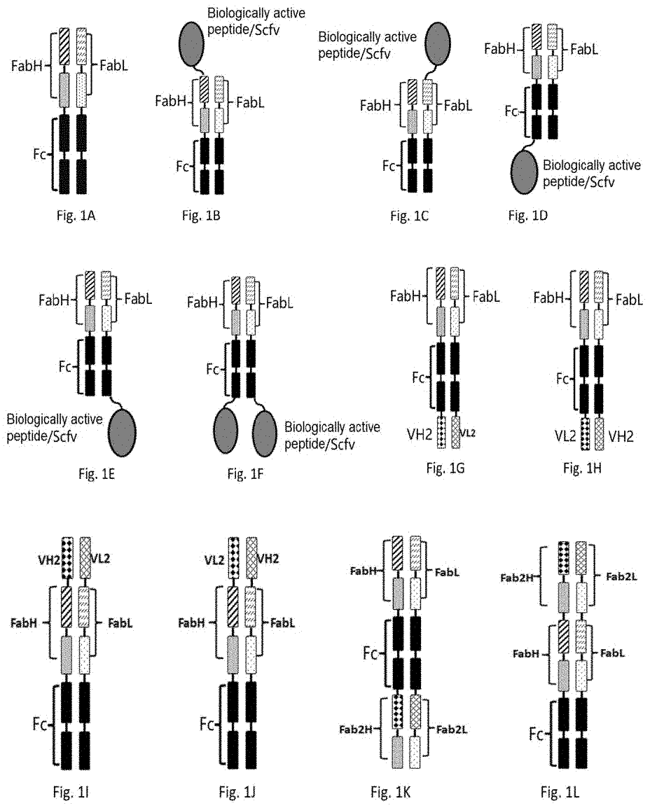

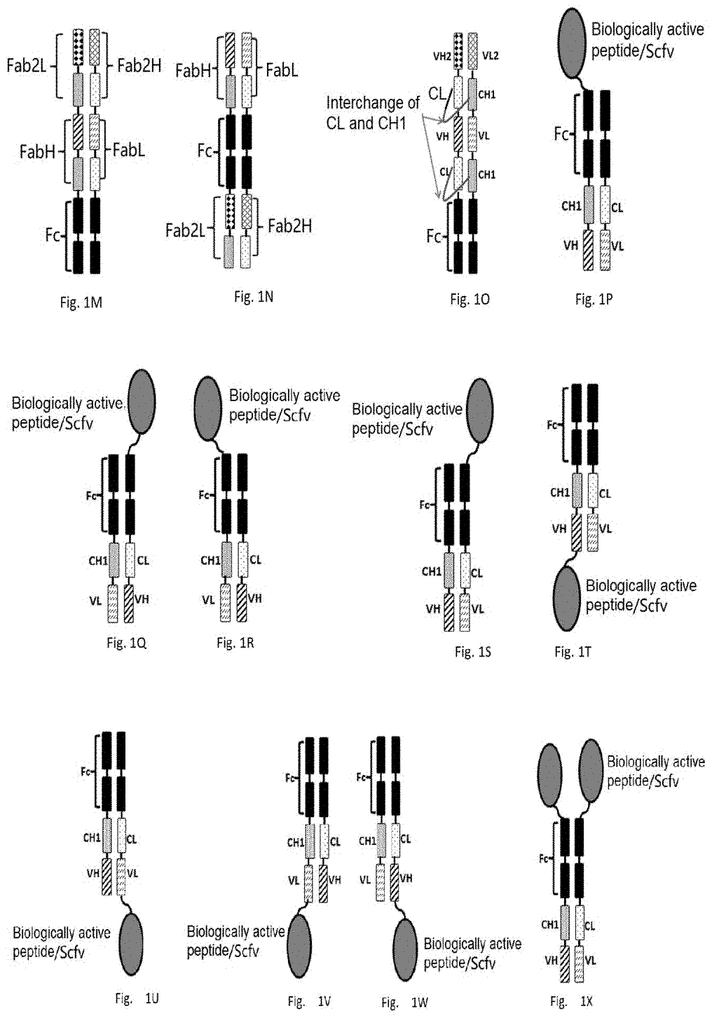

### DETAILED DESCRIPTION OF THE EMBODIMENTS

To understand the present application more comprehensively, several definitions are set forth below. Such definitions are intended to cover grammatical equivalents. The contents of all patent and literatures mentioned herein, including all sequences disclosed in these patents and disclosures, are hereby incorporated by reference.

As used herein, “heterodimeric fusion protein” means an antibody composed of two different polypeptide chains containing Fc or a fusion protein based on the antibody; where Fc of one polypeptide chain and Fc of another polypeptide chain form an Fc dimer; and two polypeptide chains form at least one antigen binding domain.

As used herein, “antigen binding domain” means a portion that specifically binds to an antigenic determinant in an antigen binding molecule. More specifically, the term “antigen binding domain” refers to a portion of an antibody which specifically binds to the partial or the whole antigen. Where the antigen is very large, the antigen binding molecule may merely bind to a specific portion of the antigen, which is called epitope. The antigen binding domain may be provided by, for example, one or more variable domains (also called a variable region). The antigen binding domain may be derived from any animal species, such as, rodent species (such as rabbit, rat or hamster) and human. Non-limiting examples of the antigen binding domain include: single-chain antibodies, Fab, F(ab′)2, Fd fragments, Fv, single chain Fv (scFv) molecules, dAb fragments, and the smallest recognition unit composed of amino acid residues that mimics the hypervariable region of an antibody. In some embodiments, the antigen binding domain is a Fab. In some embodiments, the antigen binding domain is a Fv. In some embodiments, the antigen binding domain is scfv. In some embodiments, the antigen binding domain is a biologically active peptide.

As used herein, the terms “antigen”, “antigenic determinant”, and “epitope” have the same meaning, referring to the site where the antigen polypeptide binds to the antigen binding domain to form an antigen binding domain-antigen complex (e.g., configuration formed by a segment of continuous amino acid or different regions of discontinuous amino acids). “Antigen” may be found on, for example, the surface of tumor cells, the surface of virus infected cells, the surface of other sick cells, the surface of immune cells, free substances and/or extracellular matrix (ECM) in serum. Unless otherwise specified, the protein called by antigen herein may be derived from any vertebrate, including mammal, such as, primates (e.g., human), rodent species (e.g., mouse and rat). When the specific protein is mentioned herein, the term covers “full-length” unprocessed protein, and any form of protein produced by intracellular processing. The term also covers natural protein variants, for example, splice variants or allelic mutants. In some embodiments, the antigen is a human protein. Exemplary human protein capable of being used as an antigen includes but not limited to: BCMA, CLL-1, EpCAM, CD19, CCR5, EGFR, HER2, HER3, HER4, EGF4, PSMA, CEA, MUC-1(Mucin), MUC-2, MUC-3, MUC-4, MUC-5_{AC}, MUC-5_{B}, MUC7, βhCG, Lewis-Y, CD20, CD33, CD30, CD16A, B7-H3, CD123, gpA33, P-Cadherin, GPC3, CLEC12A, CD32B, TROP-2, ganglioside GD3, 9-O-Acetyl-GD3, GM2. Globo H, fucosyl GM1, Poly SA, GD2, Carboanhydrase IX (MN/CA IX), CD44v6, Sonic Hedgehog (Shh), Wue-1, Plasma Cell Antigen, (membrane-bound) IgE, Melanoma Chondroitin Sulfate Proteoglycan (MCSP), CCR8, TNF-alpha precursor, STEAP, mesothelin, A33, Prostate Stem Cell Antigen (PSCA), Ly-6, desmoglein 4, E-cadherin neoepitope, Fetal Acetylcholine Receptor, CD25, CA19-9 marker, CA-125 marker and Muellerian Inhibitory Substance (MIS) Receptor type II, sTn (sialylated Tn antigen; TAG-72), FAP (fibroblast activation antigen), endosialin, EGFRvIII, LG, SAS, CD63, 2B4(CD244), α4β1 integrin, β2 integrin (e.g., CD11a-CD18, CD11b-CD18, CD11c-CD18), CD2(LFA2, OX34), CD16, CD27(TNFRSF7), CD38, CD96, CD100, CD160, CD137, CEACAM1(CD66), CRTAM, CS1(CD319), DNAM-1(CD226), GITR(TNFRSF18), KIR active forms (e.g., KIR2DSK KIR2DS4, KIR-S), NKG2C, NKG2D, NKG2E, natural cytotoxicity receptors (e.g., NKp30, NKp44, NKp46, NKp80), NTB-A and PEN-5, CD2(LFA2, OX34), CD3, CD5, CD27(TNFRSF7), CD28, CD30(TNFRSF8), CD40L, CD84(SLAMF5), CD137(4-1BB), CD226, CD229(Ly9, SLAMF3), CD244(2B4, SLAMF4), CD319(CRACC, BLAME), CD352(Ly108, NTBA, SLAMF6), CRTAM(CD355), DR3(TNFRSF25), GITR(CD357), HVEM(CD270), ICOS, LIGHT, LTβR(TNFRSF3), OX40(CD134), SLAM(CD150, SLAMF1), TCRα, TCRβ, TCRβγ and TIM1(HAVCR, KIM1), and the like.

Examples of tumor cell surface antigens provided by U.S. Pat. No. 7,235,641, NK cell surface antigen and more information provided in Miller, Hematology, 2013, 2013(1):247-253; Mentlik et al, Frontiers in Immunology, 2013, 4:481(1-12); Stein et al. Antibodies, 2012, 1:88-123; Pegram et al., Immunology and Cell Biology, 2011, 89:216-224; and Vivi er et al., Nature Immunology, 2008, 9:503-510; and T cell surface antigen and more information provided in Stein et al., Antibodies, 2012, 1:88-123; Chen and Flies, Nature Reviews Immunology, 2013, 13:227-242; and Pardo 11, Nature Reviews Cancer, 2012, 12:252-264, are all incorporated herein by reference.

As used herein, “Fc” is used to define the C-terminal domain of an Ig heavy chain that contains at least a part of the constant region. It means a polypeptide comprising the constant region of an antibody (exclusive of the first constant region Ig domain) and in some cases a hinge. [Jones et al, “Nature 321:522-525(1986); Riechmann et al., Nature 332:323-329(1988); and Presta, Curr. Op. Struct. BIOL. 2:529-596(1992)]. Therefore, Fc refers to the last two constant regions of human IgA, IgD and IgG, and the last three constant regions of IgE and IgM; and N-terminal of the flexible hinge of these domains. As for IgA and IgM, Fc may include the J chain. For IgG, the Fc domain contains the Ig domains Cγ2 and Cγ3 (Cγ2 and Cγ3), and the lower hinge region located between Cγ1(Cγ1) and Cγ2(Cγ2).

As used herein, “Fc variant” refers to an Fc sequence which is different from the wild Fc sequence due to at least one amino acid modification, such as substitution, deletion or insertion, but still retains the ability to pair with the corresponding Fc single chain to form an Fc dimer. In some embodiments, the amino acid modification of the “Fc variant” changes the effector function activity relative to the activity of the parent Fc region. For example, in one embodiment, the variant Fc region may have changed (i.e., increased or reduced) antibody dependent cellular cytotoxicity (ADCC), complement dependent cytotoxicity (CDC), phagocytosis, oposonization, or cell binding. In other embodiments, the Fc amino acid modification may change (i.e., increase or decrease) the affinity of the variant Fc region to FcγR relative to the wild Fc. For example, the variant Fc may have the altered affinity to FcγRI, FcγRII, and FcγRIII. In some embodiments, the variant Fc has E233P, L234V, del235L, G236A, A327G, A330S, A331S, E356D, and M358L amino acid modifications. In some embodiments, “variant Fc” is a deglycosylated Fc, that is, an Fc containing N297A amino acid modification. In other embodiments, the variant Fc further contains S354C, T366W amino acid modification, or S354C, T366W, Y349C, T366S, L368A, Y407V amino acid modification.

“Wild-type” or “WT” means an amino acid sequence or nucleotide sequence found in nature, including allelic variations herein. The WT protein has a non-intentionally modified amino acid sequence or nucleotide sequence.

As used herein, “Variant” means a polypeptide sequence which is different from its parent polypeptide sequence due to at least one amino acid modification. A variant polypeptide may refer to the polypeptide itself, a composition containing the polypeptide or an amino acid sequence encoding thereof. Preferably, compared with the parent polypeptide, the variant polypeptide has at least one amino acid modification; for example, compared with the parent polypeptide, there are about 1 to 10 amino acid modifications, preferably, about 1 to 5 amino acid modifications. Preferably, the variant polypeptide sequence and the parent polypeptide sequence herein have at least about 80% homology, more preferably at least about 90% homology, and most preferably, at least about 95% homology.

“Amino acid modification” herein means amino acid substitutions, insertions and/or deletions in the polypeptide sequence. Unless otherwise specified, the amino acid modification is generally directed to DNA-encoded amino acids, for example, the 20 amino acids having codons in DNA and RNA.

“Amino acid substitution”, or “substitution” herein means that the amino acid at a specific site in the parent polypeptide sequence is substituted by a different amino acid. To be exact, in some embodiments, the substitution is directed to non-naturally-occurring amino acids at a specific site, and these amino acids are not naturally-occurring in organisms or in any organism. For example, substitution E272Y refers to a variant polypeptide where glutamic acid at position 272 is substituted by tyrosine, and in this case, the E272Y-containing Fc is Fc variant. To be exact, the alteration of nucleic acid coding sequence while coding the same amino acid (e.g. CGG (encoding arginine) to CGA (still encoding arginine) to increase the expression level) is not “amino acid substitution”; that is, a new gene encoding the same protein is produced, but if the protein has the same amino acid, it is not an amino acid substitution. Preferably compared with the parent or natural Fc, the variant Fc of the present disclosure has conservative substitution; that is, substitution with amino acid residue having the same or similar properties. Preferably, compared with the parent or natural Fc, the variant Fc of the present disclosure has conservative substitutions of not more than 40, 30, 20, 10, or 5 as long as it can still retain the ability to pair with the corresponding Fc single chain to form Fc dimer. “Effector function” refers to biological activities attributing to the Fc region of an antibody, which differ depending on the antibody isotype. Examples of antibody effector function include: C1q binding and CDC; Fc receptor binding, ADCC, down-regulation of a cell surface receptor (e.g., B cell receptor), and B cell activation.

“ADCC” refers to cell-mediated response where the nonspecific cytotoxic cells expressing FcR (e.g., natural killer (NK) cells, neutrophil and macrophage) recognize the antibody on a target cell, and then cause the pyrolysis of the target cell. The main cells (NK cell) mediating ADCC only expresses FcγRIII, while monocyte expresses FcγRI, FcγRII, and FcγRIII. FcR expression on hematopoietic cells is summarized in table 3 of page 464 in Ravetch and Kinet, Annu. Rev. Immunol 9 (1991) 457-492.

“CDC” refers to a mechanism of inducing cell death, where the Fc effector molecular domain (one or more) of an antibody that binds to the target activates a series of enzymatic reaction, resulting in the formation of holes in the target cell membrane. Typically, an antigen-antibody complex, such as, an antigen-antibody complex on an antibody-coated target cell binds to and activates the complement C1q, which in turn activates the complement cascade, leading to the death of the target cell. The complement activation may further cause the deposition of the complement on the surface of target cells, thus facilitating ADCC by binding to complement receptor (e.g., CR3) on leukocytes.

As used herein, “Fab heavy chain” or “FabH” means polypeptide in an Ig domain containing VH and CH1; “Fab light chain” or “FabL” means polypeptide in an Ig domain containing VL and CL, namely, an Ig light chain. In some embodiments of the present disclosure, CH1 and CL in Fab can exchange positions, that is, FabH includes VH and CL, and FabL includes VL-CH1.

As used herein, “Fab” means polypeptide containing VH, CH1, VL and CL Ig domains. Fab may refer to an isolated domain, or the region in case of a full-length antibody, antibody fragment or Fab fusion protein. As is known to a person skilled in the art, Fab usually consists of two chains, Fab heavy chain and Fab light chain. In some embodiments of the present disclosure, CH1 and CL in Fab can exchange positions; therefore, the Fab in which CH1 and CL exchange positions are included in the present disclosure as well.

As used herein, “Fv” means a non-fused dimer containing a VL and a VH. “Single chain Fv” or “scfv” means polypeptide containing VL and VH domains of a single antibody, where VL and VH are on the same polypeptide.

“linkers” used herein contains one or more amino acids providing any flexible and/or rigid reasonable sequence. Preferably, the linkers is selected from a group consisting of the followings: GGGGSGGGGSGGGGS (SEQ ID NO.156), SGGGGSGGGGSGGGGS(SEQ ID NO.186), GGSGGSGGGGSGGGG(SEQ ID NO. 187), GGSGGSGGGGSGGGGS(SEQ ID NO.188), GGSGAKLAALKAKLAALKGGGGS(SEQ ID NO. 184), GGGGSELAALEAELAALEAGGSG(SEQ ID NO.185), APATSLQSGQLGFQCGELCSASA(SEQ ID NO.189), ASTKGP(SEQ ID NO.179), TVAAPSVFIFPP(SEQ ID NO. 172), PNLLGGP(SEQ ID NO. 190), GGGGS(SEQ ID NO. 152), GGGEAAAKEAAAKEAAAKAGG(SEQ ID NO. 191), G, GS, SG, GGS, GSG, SGG, GGG, GGGS, SGGG, GGGGSGS(SEQ ID NO. 153), GGGGSGGS(SEQ ID NO. 154), GGGGSGGGGS(SEQ ID NO.155), AKTTPKLEEGEFSEAR(SEQ ID NO. 157), AKTTPKLEEGEFSEARV(SEQ ID NO.158), AKTTPKLGG(SEQ ID NO. 159), SAKTTPKLGG(SEQ ID NO. 160), SAKTTP(SEQ ID NO. 162), SAKTTPKLGG(SEQ ID NO. 161), RADAAP(SEQ ID NO. 164), RADAAPTVS(SEQ ID NO. 165), RADAAAAGGPGS(SEQ ID NO. 166), RADAAAA(G4S)4, SAKTTP(SEQ ID NO. 162), SAKTTPKLGG(SEQ ID NO. 161), SAKTTPKLEEGEFSEARV(SEQ ID NO. 168), ADAAP(SEQ ID NO. 169), ADAAPTVSIFPP(SEQ ID NO. 170), TVAAP(SEQ ID NO. 171), QPKAAP(SEQ ID NO. 173), QPKAAPSVTLFPP(SEQ ID NO. 174), AKTTPP(SEQ ID NO. 175), AKTTPPSVTPLAP SEQ ID NO. 176), AKTTAP SEQ ID NO. 177), AKTTAPSV YPLAP(SEQ ID NO. 178), ASTKGPSVFPLAP(SEQ ID NO.192), GENKVEYAPALMALS(SEQ ID NO.181), GPAKELTPLKEAKVS(SEQ ID NO. 182) [symbol] GHEAAAVMQVQYPAS(SEQ ID NO. 183). The linker may also be a peptide linker that is cleavable in vivo, protease (e.g., MMP) sensitive linker, a disulfide bond-based linker that can be cleaved by reduction as mentioned above, and the like (FusionProtein Technologies for Biopharmaceuticals: Applications and Challenges, edited by Stefan R. Schmidt) or any cleavable linker known in the art.

The “biologically active peptide” in the present disclosure not only includes proteins exhibiting physiological functions in vivo after binding to an antigen binding domain, but also includes polypeptides which participate in antigen binding only but have no physiological functions. Examples of the biologically active peptide capable of being in the present disclosure may include receptors, ligand proteins, hormones, cytokines, interleukins, interleukin binding proteins, enzymes, growth factors, transcriptional regulation factors, blood coagulation factors, vaccines, structural proteins, cell surface antigens, receptor antagonists and derivatives thereof.

### EXAMPLES

For the purpose of understanding the present disclosure clearly, the above disclosure will be specifically described by reference to illustration and examples. Moreover, based on the teachings of the present disclosure, it is apparent to a person skilled in the art to make some changes and modifications to the present disclosure within the spirit and scope of the claims attached herein. The examples below are only provided in a manner that does not restrict it. A person skilled in the art will easily recognize multiple non-key parameters, which may be change or modified to obtain basically similar results.

For all constant region positions discussed in the present disclosure, the numbering rule shall be in accordance with the EU index as in Rabat (Rabat, et al. 1991, Sequences of proteins of Immunological Interest, 5th Ed., United States Public Health Service, National Institutes of Health, Bethesda, incorporated by reference herein in its entirety). A person skilled in the art of antibodies will understand that this convention consisting of non-sequential numbering in the specific regions of an Ig sequence can be standardized with reference to the conservative positions in the Ig family. Therefore, the position of any given Ig defined by the EU index does not necessarily corresponds to its sequential sequence.

### Example 1 Construction of Eukaryotic Expression Vectors of Heterodimeric Fusion Proteins

### 1.1 Gene Synthesis

anti-CD19 VH, anti-CD19 VL, anti-CD3 VH-1, anti-CD3 VL-1, anti-CD3 VH-3, anti-CD3 VL-3, anti-EGFR VH, anti-EGFR VL, biologically active peptide EGF4, biologically active peptide NKG2D, anti-CD3 VH-4, anti-CD3 VL-4, anti-BCMA VH, anti-BCMA VL, anti-CLL-1 VH, anti-CLL-1 VL, a light chain CL of an antibody and a heavy chain CH1 gene of an antibody (synthesized by IDT) were synthesized.

1.2 scfv Construction

The above gene segments were amplified by PCR, and the amplified anti-CD3 VH-1 and anti-CD3 VL-1, anti-EGFR VH and anti-EGFR VL, anti-CD3 VL-3 and anti-CD3 VH-3, anti-CD19 VH and anti-CD19 VL, anti-BCMA VH and anti-BCMA VL, anti-CLL-1 VH and anti-CLL-1 VL were individually linked via a linker through overlap PCR to obtain scfv1(anti-CD3 scfv-1), scfv2(anti-EGFR scfv), scfv3(anti-CD3 scfv-3), scfv5(anti-CD19 scfv), scfv6(anti-BCMA scfv), and scfv7(anti-CLL-1 scfv) fragments, and then sequencing was performed for verification.

### 1.3 Fab Fragment Construction

Gene segments synthesized in 1.1 were amplified by PCR respectively, and the PCR products anti-CD3 VL-1, anti-EGFR VL, anti-CD3 VL-3 and anti-CD3 VL-4 were individually linked with CL′ to obtain anti-CD3 VL-1-CL(Fab1L), anti-EGFRVL-CL(Fab2L), anti-CD3 VL-3-CL(Fab3L), and anti-CD3 VL-4-CL(Fab4L), and then sequencing was performed for verification.

Gene segments synthesized in 1.1 were amplified by PCR respectively, and the amplified anti-CD3 VH-1, anti-EGFR VH, anti-CD3 VH-3, and anti-CD3 VH-4 were individually connected with CH1 via overlap PCR to obtain anti-CD3 VH-1-CH1(Fab1H), anti-EGFR VH-CH1(Fab2H), anti-CD3 VH-3-CH1(Fab3H), and anti-CD3 VH-4-CH1(Fab4H), and then sequencing was performed for verification.

The above sequencing-verified FabH and FabL were further connected via in-frame and cloned into a pFuse-hIgG1-Fc2 vector (InvivoGen, Calif.), respectively, and the Fc fragment on the vector had the following mutations: N297A or E233P, L234V, L235A, delG236, A327G, A330S, A331S. In some embodiments, Fc further includes S354C, T366W mutation or Y349C, T366S, L368A, Y407V mutation. According to demands, scfv or biologically active peptide or VL or Fab was connected to the above vector via a linker, and all the sequences constructed were subjected to sequencing for verification. Nucleotide and amino acid sequences of each construct were shown in Sequence Listing: Seq No. 1-Seq. No. 142.

TABLE 1
Construct and serial number

| | | Nucleotide | Protein |
|---|---|---|---|
| | | sequence | sequence |
| Construct | | Seq ID No: | Seq ID No: |
| IgFD-1(anti-CD19/CD3) | Chain-1 | 91 | 92 |
| | Chain-2 | 93 | 94 |
| IgFD-2(anti-CD19/CD3) | Chain-1 | 95 | 96 |
| | Chain-2 | 97 | 98 |
| IgFD-3(anti-CD19/CD3) | Chain-1 | 99 | 100 |
| | Chain-2 | 101 | 102 |
| IgFD-4(anti-CD19/CD3) | Chain-1 | 1 | 2 |
| | Chain-2 | 3 | 4 |
| IgFD-5(anti-CD19/CD3) | Chain-1 | 19 | 20 |
| | Chain-2 | 21 | 22 |
| IgFD-6(anti-CD19/CD3) | Chain-1 | 5 | 6 |
| | Chain-2 | 7 | 8 |
| IgFD-7(anti-CD19/CD3) | Chain-1 | 23 | 24 |
| | Chain-2 | 25 | 26 |
| IgFD-8(anti-CD19/CD3) | Chain-1 | 23 | 24 |
| | Chain-2 | 27 | 28 |
| IgFD-9(anti-CD3/EGFR) | Chain-1 | 59 | 60 |
| | Chain-2 | 61 | 62 |
| IgFD-10(anti-CD3/EGFR) | Chain-1 | 71 | 72 |
| | Chain-2 | 73 | 74 |
| IgFD-11(anti-CD3/EGFR) | Chain-1 | 29 | 30 |
| | Chain-2 | 31 | 32 |
| IgFD-12(anti-CD3/EGFR) | Chain-1 | 63 | 64 |
| | Chain-2 | 65 | 66 |
| IgFD-13(anti-CD3/EGFR) | Chain-1 | 75 | 76 |
| | Chain-2 | 77 | 78 |
| IgFD-14(anti-CD3/EGFR) | Chain-1 | 83 | 84 |
| | Chain-2 | 85 | 86 |
| IgFD-15(anti-CD3/EGFR) | Chain-1 | 87 | 88 |
| | Chain-2 | 89 | 90 |
| IgFD-16(anti-CD3/EGFR) | Chain-1 | 29 | 30 |
| | Chain-2 | 33 | 34 |
| IgFD-17(anti-CD3/EGFR) | Chain-1 | 9 | 10 |
| | Chain-2 | 11 | 12 |
| IgFD-18(anti-CD3/EGFR) | Chain-1 | 17 | 18 |
| | Chain-2 | 11 | 12 |
| IgFD-19(anti-CD3/EGFR) | Chain-1 | 29 | 30 |
| | Chain-2 | 57 | 58 |
| IgFD-20(anti-CD3/EGFR) | Chain-1 | 35 | 36 |
| | Chain-2 | 37 | 38 |
| IgFD-21(anti-CD3/EGFR) | Chain-1 | 13 | 14 |
| | Chain-2 | 15 | 16 |
| IgFD-22(anti-CD3/BCMA) | Chain-1 | 29 | 30 |
| | Chain-2 | 39 | 40 |
| IgFD-23(anti-CD3/CLL-1) | Chain-1 | 29 | 30 |
| | Chain-2 | 41 | 42 |
| IgFD-25(anti-CD3/EGFR) | Chain-1 | 43 | 44 |
| | Chain-2 | 45 | 46 |
| IgFD-26(anti-CD3/EGFR) | Chain-1 | 47 | 48 |
| | Chain-2 | 49 | 50 |
| IgFD-28(anti-CD3/EGFR) | Chain-1 | 43 | 44 |
| | Chain-2 | 51 | 52 |
| IgFD-29(anti-CD3/EGFR) | Chain-1 | 67 | 68 |
| | Chain-2 | 69 | 70 |
| IgFD-30(anti-CD3/EGFR) | Chain-1 | 79 | 80 |
| | Chain-2 | 81 | 82 |
| IgFD-31(anti-CD3/EGFR) | Chain-1 | 53 | 54 |
| | Chain-2 | 55 | 56 |
| IgFD-32(anti-CD3/EGFR) | Chain-1 | 103 | 104 |
| | Chain-2 | 105 | 106 |
| IgFD-33(anti-CD3/EGFR) | Chain-1 | 107 | 108 |
| | Chain-2 | 109 | 110 |
| IgFD-34(anti-CD3/EGFR) | Chain-1 | 111 | 112 |
| | Chain-2 | 113 | 114 |
| IgFD-35(anti-CD3/EGFR) | Chain-1 | 115 | 116 |
| | Chain-2 | 117 | 118 |
| IgFD-36(anti-CD3) | Chain-1 | 43 | 44 |
| | Chain-2 | 119 | 120 |
| IgFD-37(anti-CD3/MICA) | Chain-1 | 121 | 122 |
| | Chain-2 | 123 | 124 |
| Fab1H(anti-CD3 VH-1-CH1) | / | / | 125 |
| Fab1L(anti-CD3 VL-1-CL) | / | / | 126 |
| Fab2H(anti-EGFR VH-CH1) | / | / | 127 |
| Fab2L(anti-EGFR VL-CL) | / | / | 128 |
| Fab3H(anti-CD3 VH-3-CH1) | / | / | 129 |
| Fab3L(anti-CD3 VL-3-CL) | / | / | 130 |
| Fab4H(anti-CD3 VH-4-CH1) | / | / | 131 |
| Fab4L(anti-CD3 VL-4-CL) | / | / | 132 |
| Fv1(anti-CD3 Fv) | Chain-1 | / | 133 |
| | Chain-2 | / | 134 |
| Fv2(anti-EGFR Fv) | Chain-1 | / | 135 |
| | Chain-2 | / | 136 |
| Fv3(anti-CD3 -3 Fv) | Chain-1 | / | 137 |
| | Chain-2 | / | 138 |
| scfv5(anti-CD19 scfv) | / | / | 139 |
| scfv6(anti-BCMA scfv) | / | / | 140 |
| scfv7(anti-CLL-1 scfv) | / | / | 141 |
| scfv2(anti-EGFR scfv) | / | / | 142 |
| FcV1 | / | / | 143 |
| FcV2 | / | / | 144 |
| FcV3 | / | / | 145 |
| FcV4 | / | / | 146 |
| FcV5 | / | / | 147 |
| FcV6 | / | / | 148 |
| FcWT | / | / | 149 |
| EGF4 | / | / | 150 |
| NKG2D | / | / | 151 |

### Example 2 Expression, Purification and Size Exclusion Chromatography of the Exemplary Heterodimeric Fusion Proteins

Heterodimeric fusion proteins were expressed through transient transfection of FreeStyle HEK 293 cells (ThermoFisher) with expression vectors, according to the manufacturer's protocol. Briefly, 28 ml FreeStyle HEK293 (3×10⁷ cell/ml) were seeded in a 125 ml shaking flask, plasmids of two chains diluted by 1 ml Opti-MEM (Invitrogen) were added to 1 ml Opti-MEM containing 60 μl 293 Fectin(Invitrogen, Inc). After the plasmids were incubated for 30 min at room temperature, the plasmid-293fectin mixture was added to the cell suspension. Cells were shaken at 125 rpm, 37° C., 5% CO₂. Cell culture supernatant was collected 48 h and 96 h after transfection, respectively. The heterodimeric fusion proteins was purified by CH1 Select resin(Thermo Fisher Scientific, IL), Protein G and/or Protein A Resin (Genscript) according to the instructions of manufacturers. The composition and purity of the purified heterodimeric fusion proteins were analyzed under reducing conditions and non-reducing conditions by SDS-PAGE. Protein concentration was measured by A280 and BCA (Pierce, Rockford, Ill.).

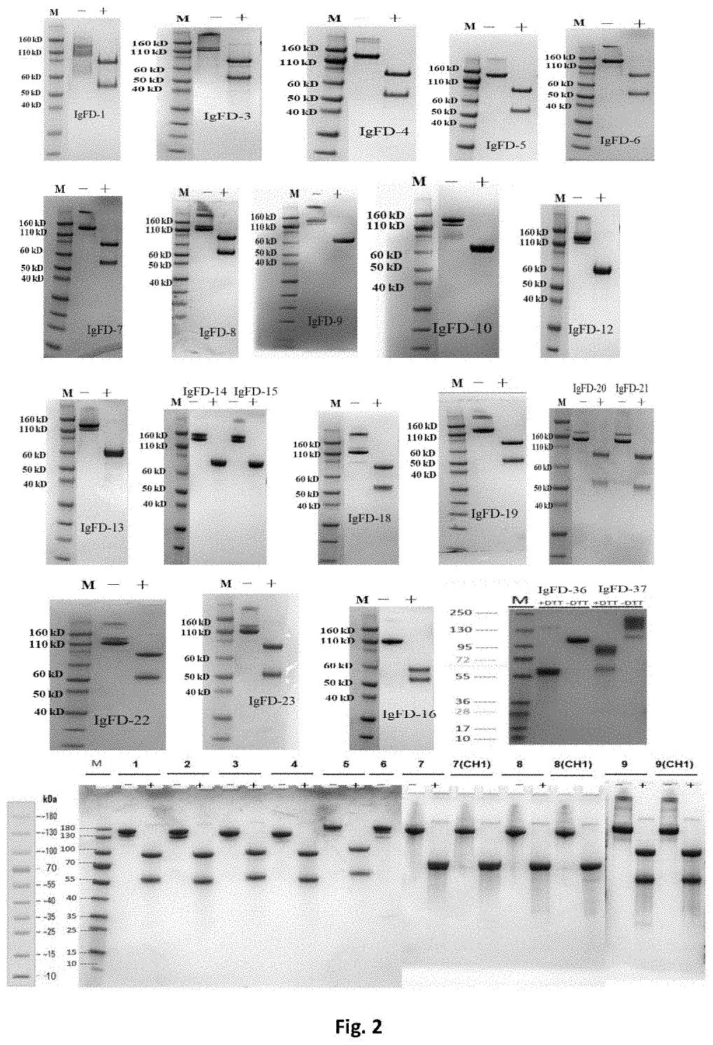

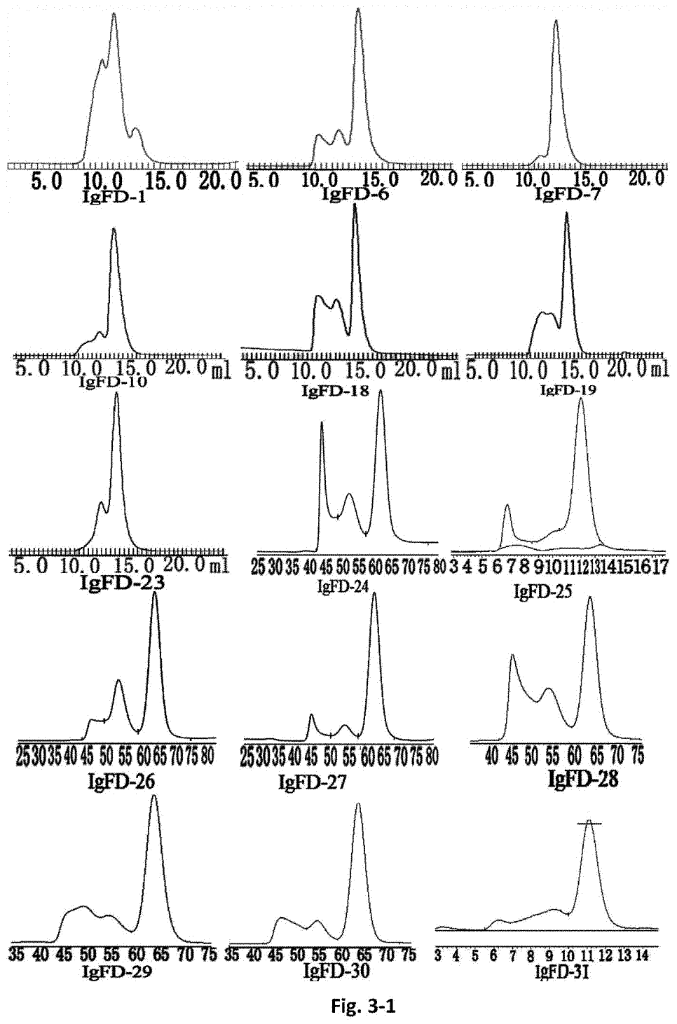

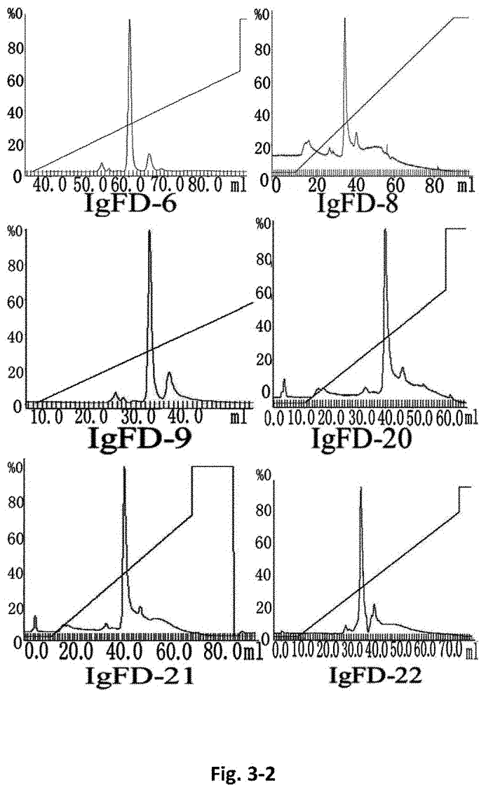

The heterodimeric fusion proteins purified by CH1 resin, Protein and/or Protein A resin were subjected to GE's AKTA chromatography column; and the chromatographic column used was: Superdex 200 Increase 10/300 GL size exclusion chromatography column and/or Mono S 5/50 GL ion exchange chromatographic column. The solution used for the size exclusion chromatography was PBS buffer solution (0.010 M phosphate buffer, 0.0027M KCl, 0.14 M NaCl, pH 7.4); and the solution for ion exchange chromatography was Buffer A:20 mM NaOAc, pH=5 and Buffer B:20 mM NaOAc, 1 M NaCl, pH=5. It can be seen from SDS-PAGE of FIG. 2, chromatogram of FIG. 3, and protein expression results of Table 2 that the expression of different heterodimeric fusion proteins had considerable purity, and the expression level was equivalent to that of the conventional mAb, indicating that these heterodimeric antibodies can be highly expressed in mammalian cells.

TABLE 2
Analysis on protein expression

| Protein | Expression level (mg/L) |
|---|---|
| IgFD-6 | 10.7 |
| IgFD-21 | 3.75 |
| IgFD-22 | 4.58 |
| IgFD-23 | 8.3 |
| IgFD-9 | 30 |
| IgFD-10 | 25.3 |
| IgFD-33 | 18 |

### Example 3 Mass Spectrometry of Heterodimeric Antibodies

Samples purified by size exclusion chromatography were incubated with PNGase F(NEB) for 8 h at 37° C., and 10 mM DTT was added for processing, then the sample was injected into a 300SB-C8, 2.1×50 mm column of HPLC-Q-TOF-MS (Agilent, USA) for MS analysis. The result was shown in Table 3; the theoretically predicted molecular weight of two chains of the different heterodimeric fusion proteins was basically consistent with the molecular weight measured by mass spectrometric detection.

TABLE 3
Analysis on protein mass spectrometry

| | Chain-1 | | Chain-2 | | Whole protein | |
|---|---|---|---|---|---|---|
| | Theoretical | Detected | Theoretical | Detected | Theoretical | Detected |
| | Mass (D) | Mass (D) | Mass (D) | Mass (D) | Mass (D) | Mass (D) |
| IgFD-1 | 78322.68 | 78372.98 | 49136.05 | 4913.77 | / | / |
| IgFD-4 | 76515.64 | 76507.13 | 48848.77 | 48868.93 | / | / |
| IgFD-5 | 49444.77 | 49436.06 | 75919.64 | 75911.44 | / | / |
| IgFD-6 | 76886.98 | 76877.355 | 48631.67 | 48608.096 | / | / |
| IgFD-7 | 49816.11 | 49810.185 | 75702.54 | 75676.992 | / | / |
| IgFD-8 | 49816.11 | 49808.9 | 75769.02 | 75244.7 | / | / |
| IgFD-9 | 63914.7 | 63906.4 | 61385.68 | 61359.6 | / | / |
| IgFD-10 | 62570.13 | 62560.7 | 62730.26 | 62704.1 | / | / |
| IgFD-14 | 75197.52 | 75185.7 | 73274.97 | 73230.8 | 148440.49 | 148406.6 |
| IgFD-15 | 74459.41 | 74446.7 | 74013.08 | 73982.9 | 148440.49 | 148423.6 |
| IgFD-16 | 49958.37 | 49950 | 55979.98 | 55685.5 | 105916.48 | 105622.8 |
| IgFD-18 | 76467.55 | 76455.2 | 48773.93 | 48748.8 | 125209.48 | 125197.1 |
| IgFD-19 | 49958.37 | 49949.50 | 75283.1 | 75270.6 | 125215.47 | 125214.09 |
| IgFD-20 | 76611.76 | 76875.7 | 48430.59 | 48501.5 | / | / |
| IgFD-21 | 49974.41 | 50132.2 | 75067.93 | 75237.6 | / | / |
| IgFD-22 | 49958.37 | 49950.1 | 77112.31 | 77083.2 | / | / |
| IgFD-23 | 49958.37 | 49950.0 | 75319.2 | 75289.7 | / | / |
| IgFD-25 | 49917.32 | 49911.87 | 75088.89 | 75063.08 | / | / |
| IgFD-26 | 50059.59 | 50051.62 | 75231.16 | 75202.31 | 125290.75 | 125245.79 |

### Example 4 In Vitro Activity Studies

### 4.1 Analysis of In Vitro Activity of the Anti-CD3/CD19 Heterodimeric Fusion Proteins

### (1) Analysis of the Binding of the Anti-CD3/CD19 Heterodimeric Fusion Proteins to NALM-6 Cells by Flow Cytometry.

NALM6 cells (a RPMI1640 medium containing 10% FBS) were cultured, and 2×10⁵ cells were taken and washed by pre-cooled PBS for 3 times, and blocked by 2% FBS (in PBS), and then incubated with purified IgFD-6 or IgFD-7 at different concentrations (200 nM, 40 nM, 8 nM, 1.6 nM) for 2 h at 4° C. (cells were mixed gently during incubation). Unbounded antibodies were washed by 2% FBS (soluble to PBS), and then the remaining cells were incubated with FITC anti-human IgG Fc(KPL, Inc., MD) for 1 h at 4° C., washed by 2% FBS (soluble to PBS), and subjected for flow cytometry analysis.

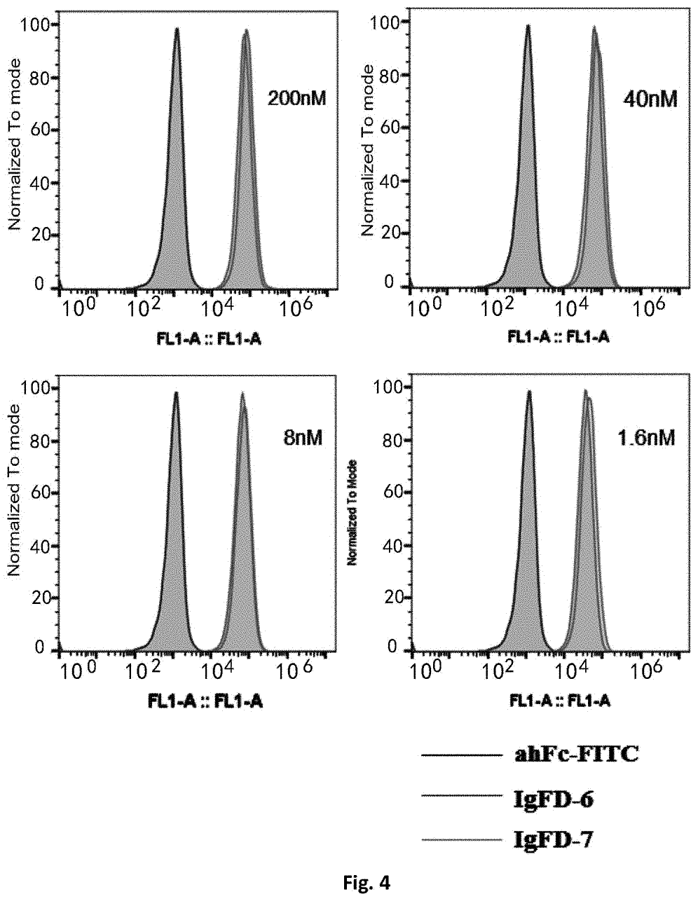

The result was shown in FIG. 4. Compared with the control (PBS+FITC anti-human IgG Fc), IgFD-6 and IgFD-7 at different concentrations had stronger binding to CD19+NALM6 cells, and the higher the concentration was, the stronger the binding capacity was.

### (2) Specific Killing Effect of PBMCs on NALM-6 Cells Medidated by Anti-CD3/Anti-CD19 Heterodimeric Fusion Proteins

Peripheral blood of healthy volunteers was collected, and Ficoll-Hypaque(GE Healthcare) was used to separate peripheral blood monouclear cells (PBMCs) via gradient centrifugation. Purified PBMCs were resuspended in RPMI 1640/10% FBS complete medium and incubated with immobilized anti-CD3 (Clone OKT3, eBiosciences), and 2 μg mL anti-CD28 antibodies (Clone CD28.2, eBiosciences) at 37° C. for 48 h. The activated T cells were then expanded for 10d with 20 U/ml IL2(R&D Systems).

NALM6 cells (a RPMI1640 medium containing 10% FBS) were cultured, and labeled by Green fluorescent cell linker mini kit (Sigma). Then 10⁴ labeled NALM6 cells were incubated with the above activated T cells at a ratio of 1:5 (NALM6 cells were 10⁴, and T cells were 5*10⁴). Gradiently diluted IgFD-6 or IgFD-7 w was incubated with the cell n mixture for 24 h at 37° C., and 1%7-AAD was added for analysis by an FCM. Cells having positive green fluorescence/negative 7-AAD were vital NALM6 cells.

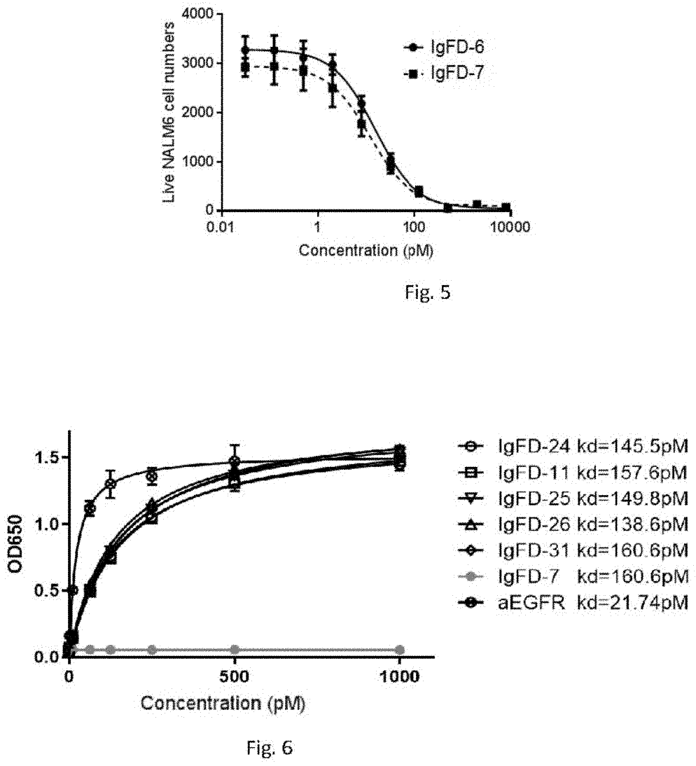

The result was shown in FIG. 5. IgFD-6 and IgFD-7 can effectively mediate the killing effect of T cells in PBMCs on Nalm6 cells.

### 4.2 Analysis of the In Vitro Activity of an Anti-CD3/EGFR Heterodimer

### (1) Analysis of the Binding of the Anti-CD3/EGFR Heterodimer to Human EGFR by ELISA

hEGFR-6-his (SinoBiological) (100 ng/well) was coated in a 96-well plate, and incubated overnight at 4° C. After blocked by PBST (0.5% Tween-20 in PBS) containing 2% skim milk powder for 1 h at room temperature, the gradiently diluted heterodimeric antibodies IgFD-11, IgFD-24, IgFD-25, IgFD-26, IgFD-31, and anti-EGFR were added for incubation for 2 h at room temperature, respectively. After washing with PBST containing 2% skim milk powder for 4-5 times, an Anti-Human IgG (FC) Antibody-HRP (KPL) secondary antibody was added for incubation for 1 h at room temperature, and then washed by PBST containing 2% skim milk powder for 4-5 times, reading was performed at 650 nm after color development with a TMB colour reagent (BioLegend, Cat. 421101). As shown in FIG. 6, the anti-CD3/anti-EGFR heterodimer of different fusion forms had stronger binding to human EGFR.

### (2) Analysis of the Binding of the Anti-CD3/Anti-EGFR Heterodimer to F98-EGFR Cells by Flow Cytometry

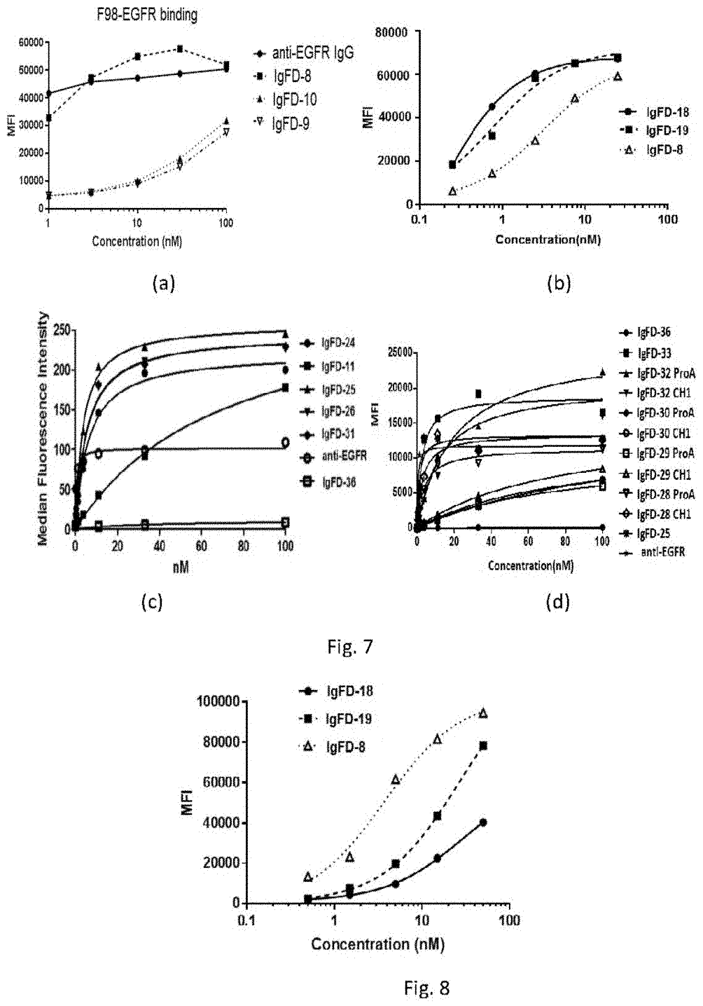

F98-EGFR cells (a DMEM containing 10% FBS, 200 μg/ml G418) were cultured. 2×10⁵ cells were washed by precooled PBS for 3 times, and blocked by 2% FBS (soluble to PBS). IgFD-8, IgFD-9, IgFD-10, IgFD-11, IgFD-18, IgFD-19, IgFD-25, IgFD-26 or IgFD-31 (aEGFR expressed in a laboratory as a control) was added and incubated for 2 h at 4° C. (cells were gently mixed during incubation), unbounded antibodies were washed by 2% FBS (soluble to PBS), and then the remaining cells were incubated by FITC anti-human IgGFc(KPL, Inc., MD) for 1 h at 4° C., and washed by 2% FBS (soluble to PBS) for flow cytometry analysis. As shown in FIG. 7 and Table 4, the anti-CD3/anti-EGFR heterodimers in different forms of fusion could bind to EGFR on F98-EGFR cells, and there was no significant difference in the binding capacity of different heterodimers to F98-EGFR.

(3) Analysis of the Binding of the Anti-CD3/Anti-EGFR Heterodimers to T Cells from PBMCs

Peripheral blood of healthy volunteers was collected, and Ficoll-Hypaque(GE Healthcare) was used to separate peripheral blood monouclear cells (PBMCs) via gradient centrifugation. The purified PBMCs were resuspended in RPMI 1640/10% FBS complete medium. PBMCs were incubated with immobilized anti-CD3 (Clone OKT3, eBiosciences), and 2 μg mL anti-CD28 (Clone CloneCD28.2, eBiosciences) at 37° C. for 48 h. The activated T cells were then expanded for 10d with 20 U/ml IL2(R&D Systems). 2×10⁵ cells were washed by precooled PBS for 3 times, and blocked by 2% FBS (in PBS), and then incubated with IgFD-8, IgFD-18 or IgFD-19 at different concentrations (25 nM, 7.5 nM, 2.5 nM, 0.75 nM or 0.25 nM) for 1 h at 4° C. (cells were gently mixed during incubation). Unbounded antibodies were washed by 2% FBS (in PBS), and the cells were incubated with FITC anti-human IgGFc (KPL, Inc., MD) for 1 h at 4° C. (cells were gently mixed during incubation). The cells were subjected for flow cytometry analysis after the unbounded antibodies were washed by 2% FBS (in PBS).

The result was shown in FIG. 8. The binding capacity of the heterodimer in different forms of first ion to PBMC-T cells was: IgFD-8>IgFD-19>IgFD-18; and the binding capacity thereof to F98-EGFR cells was: IgFD-18>IgFD-19>IgFD-8 (FIG. 6); indicating that the affinity of scfv (anti-EGFRscfv and anti-CD3 scfv) to antigen was stronger than that of the Fab (anti-EGFRFab and anti-CD3 Fab).

### (4) Analysis of the Binding of the Anti-CD3/EGFR Heterodimers to Jurkat Cells

Jurkat cells (a medium containing 10% FBS) were cultured. 2×10⁵ cells were washed by precooled PBS for 3 times, and blocked by 2% FBS (soluble to PBS). Purified IgFD-11, IgFD-8, IgFD-25, IgFD-26, IgFD-31 or IgFD-36 (aEGFR expressed in a laboratory as a control) were added and incubated for 2 h at 4° C. (cells were gently mixed during incubation), followed by 2% FBS(in PBS) wash. The cells were incubated by FITC anti-human IgGFc (KPL, Inc., MD) for 1 h at 4° C. and subjected to flow cytometry analysis after washing with 2% FBS(in PBS).

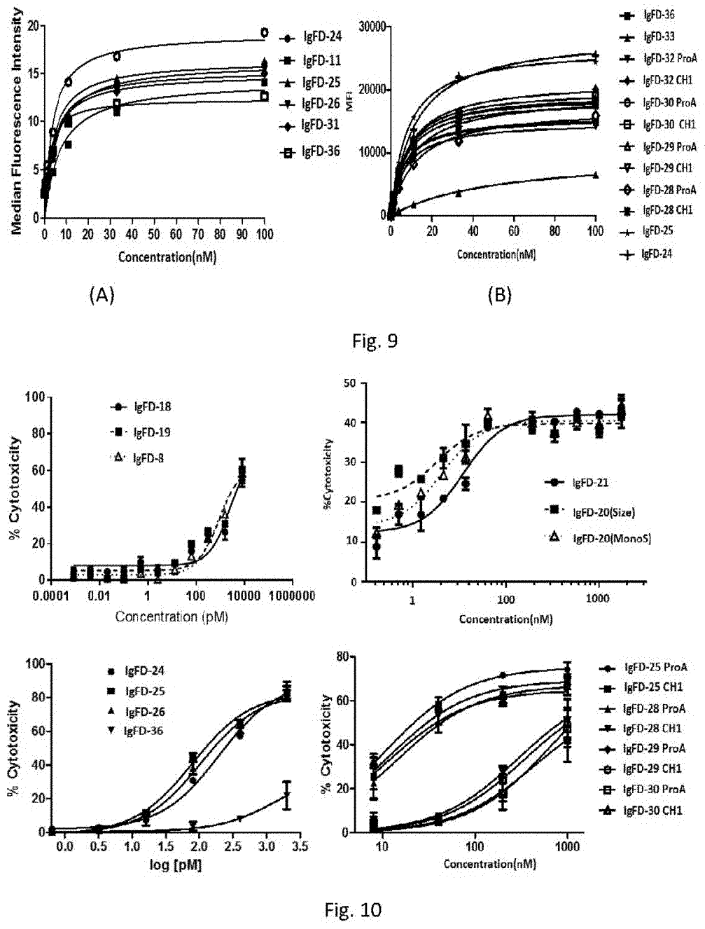

The result was shown in FIG. 9. The anti-CD3/EGFR heterodimer in different forms of fusion could bind to Jurkat T cells well.

### (5) Analysis of the Specific Killing Effect of PBMCs on F98-EGFR Cells Mediated by the Anti-CD3/EGFR Heterodimers Detected by LDH Assay.

Peripheral blood of healthy volunteers was collected, and Ficoll-Hypaque(GE Healthcare) was used to separate peripheral blood monouclear cells (PBMCs) via gradient centrifugation. Isolated PBMCs were resuspended in RPMI 1640/10% FBS complete medium and then incubated with immobilized anti-CD3 (Clone OKT3, eBiosciences), and 2 μg mL anti-CD28 (Clone CD28.2, eBiosciences) at 37° C. for 48 h. The activated T cells were then expanded for 10d with 20 U/ml IL2(R&D Systems).

F98-EGFR cells (a DMEM medium containing 10% FBS, 200 μg/ml G418) were cultured, and 10⁴ were incubated with the above activated T cells at a ratio of 1:5 (F98-EGFR cells were 10⁴, and T cells were 5*10⁴). Gradiently diluted IgFD-8, IgFD-18, IgFD-19, IgFD-21, IgFD-20, IgFD-25, IgFD-26, IgFD-28, IgFD-29 or IgFD-30 were added for incubation for 24 h at 37° C. LDH levels cin culture supernatant was detected by Cytotox-96 nonradioactive cytotoxicity assay kit (Promega). OD values at 490 nm were read by SpectraMax 250. Cytotoxicity (represented by %) was calculated as follows:

% Cytotoxicity=(Experimental−Effector Spontaneous−Target Spontaneous)/(Target Maximum−Target Spontaneous)×100

where, Target Maximum was the LDH content in supernatant where only F98-EGFR cells were lysed.
Target Spontaneous was the LDH content in supernatant where there were only F98-EGFR cells.
Effector Spontaneous was the LDH content in supernatant where there were only effector cells (T cells).

The result was shown in FIG. 10. The anti-EGFR&anti-CD3 in different forms of fusion could effectively recruit T cells in PBMCs to produce corresponding killing effect on F98-EGFR cells.

### 4.3 Analysis of the Killing Effect of PBMC on MM1.R Cells Mediated by the Anti-CD3/BCMA Heterodimer

Peripheral blood of healthy volunteers was collected, and Ficoll-Hypaque(GE Healthcare) was used to separate peripheral blood monouclear cells (PBMCs) via gradient centrifugation. Purified PBMCs were resuspended on a RPMI 1640/10% FBS complete medium and incubated with immobilized anti-CD3 (Clone OKT3, eBiosciences), and 2 μg/mL anti-CD28 (Clone CloneCD28.2, eBiosciences) at 37° C. for 48 h. The activated T cells were then expanded for 10d with 20 U/ml IL2(R&D Systems).

### (1) LDH Assay:

MM1.R cells (a RPMI1640 medium containing 10% FBS) were cultured, and 10⁴ were incubated with the above activated T cells at a ratio of 1:5 (MM1.R cells were 10⁴, and T cells were 5*10⁴). Gradiently diluted IgFD-22 was added and incubated for 24 h at 37° C.

LDH level in culture supernatant was detected by Cytotox-96 nonradioactive cytotoxicity assay kit (Promega). OD values at 490 nm were read by SpectraMax 250. Cytotoxicity (represented by %) was calculated as follows:

% Cytotoxicity=(experimental−Effector Spontaneous−Target Spontaneous)/(Target Maximum−Target Spontaneous)×100

where, Target Maximum was the LDH content in supernatant where only MM1.R cells were lysed.
Effector Spontaneous was the LDH content in supernatant where there were only MM1.R cells.
Effector Spontaneous was the LDH content in supernatant where there were only effector cells (T cells).

### (2) Flow Cytometry Analysis

MM1.R cells (a RPMI 1640 medium containing 10% FBS) were cultured, and labeled by Green fluorescent cell linker mini kit (Sigma). 10⁴ labeled MM1.R cells were incubated with the above activated T cells at a ratio of 1:5 (MM1.R cells were 10⁴, and T cells were 5*10⁴). Gradiently diluted IgFD-22 was added for incubation for 24 h at 37° C., and then 1% 7-AAD was added and subjected for analysis by flow cytometry. Cells having positive green fluorescence/negative 7-AAD were vital MM1.R cells.

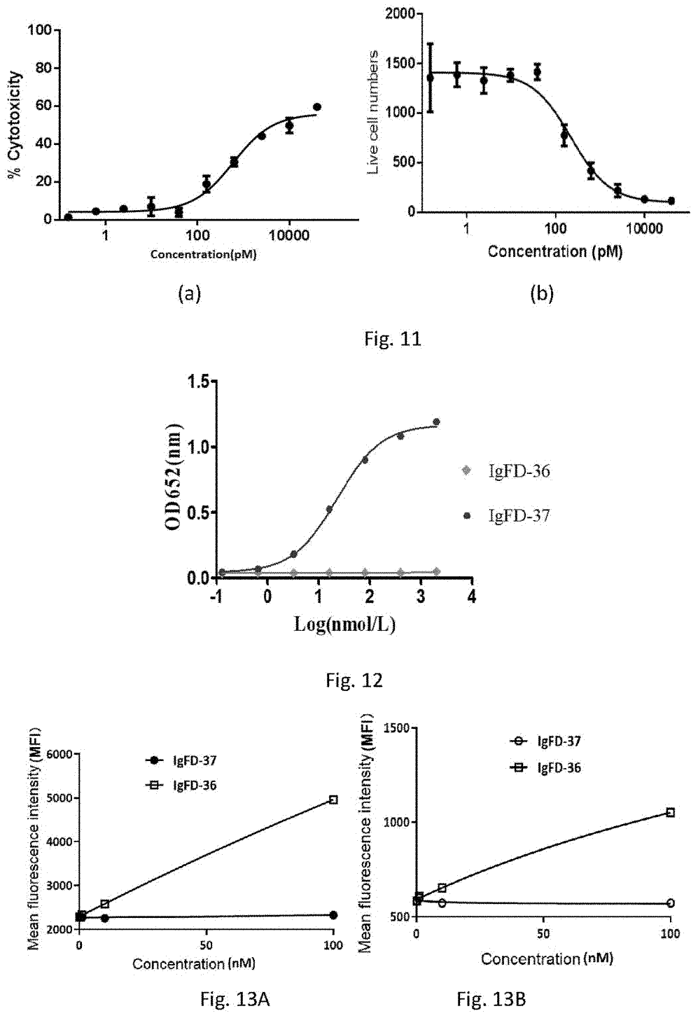

LDH assay and flow cytometry results were shown in FIG. 11 (A) and FIG. 10 (B). IgFD-22 could effectively mediate the killing effect of PMBCs on MM1.R cells.

4.4 Evaluation of the In Vitro Activity of the anti-CD3/MICA Heterodimer

### (1) Analysis of the Binding of the Anti-CD3/MICA Heterodimer to Human MICA by ELISA

MICA (Sino Biological Inc.) (100 ng/well) was coated in a 96-well plate, and incubated overnight at 4° C. After blocking by PBST (0.5% Tween-20 in PBS) containing 2% skim milk powder for 1 h at room temperature, the gradiently diluted heterodimeric antibodies IgFD-36 and IgFD-37 were added for incubation for 1 h at 37° C., and washed by PBST containing 2% skim milk powder for 3 times. Afterwards, an anti-human Fc-HRP secondary antibody (KPL 5200-0279, 1:1000) was added for incubation for 1 h at room temperature, and then washed by PBST containing 2% skim milk powder for 5 times. Readings at 652 nm were performed with TMB color reagent (BioLegend, Cat. 421101). As shown in FIG. 12, the IgFD-37 heterodimer had stronger binding capacity to human MICA.

### (2) Analysis of the Binding of the Anti-CD3/MICA Targeting Heterodimer to PANC-1, BXPC-3, and K562 by FCM

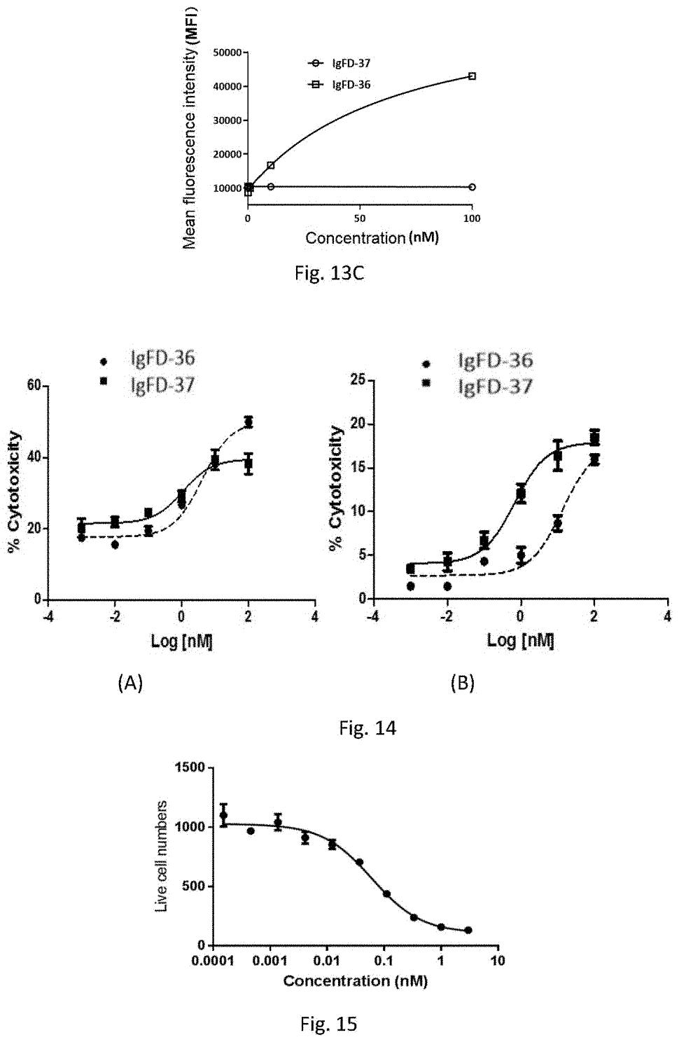

PANC-1 (a DMEM medium containing 10% FBS), BXPC-3 (a RPMI1640 medium containing 10% FBS), and K562 cells (a RPMI1640 medium containing 10% FBS) were cultured, respectively. 2×10⁵ cells were washed by pre-cooled PBS for 3 times, and blocked by 2% FBS (soluble to PBS), and then incubated with purified IgFD-36 or IgFD-37 for 2 h at 4° C. (cells were gently mixed during incubation) followed by washed by 2% FBS (in PBS). Then cells were incubated with anti-human kappa light chain-FITC (Biolegend, 316506) for 1 h at 4° C. and subjected to flow cytometry analysis after washing by 2% FBS (in PBS). As shown in FIG. 13 and Table 4, the heterodimer targeting CD3 and MICA could bind to NKG2DL subunit MICA on PANC-K BXPC-3 or K562 cells.

### (3) Analysis of the Specific Killing Effect of PBMCs on Cells Expressing MICA by the Anti-CD3/MICA Heterodimer

Peripheral blood of healthy volunteers was collected, and Ficoll-Hypaque(GE Healthcare) was used to separate peripheral blood monouclear cells (PBMCs) via gradient centrifugation. Purifieded, PBMCs were resuspended in RPMI 1640/10% FBS complete medium and incubated with immobilized anti-CD3 (Clone OKT3, eBiosciences), and 2 μg/mL anti-CD28 (Clone CD28.2, eBiosciences) at 37° C. for 48 h. The activated T cells were then expanded for 10d with 20 U/ml IL2(R&D Systems).

K562/PANC-1 cells were cultured, and 10⁴ were incubated with the above activated T cells at a ratio of 1:5 (K562 or PANC-1 cells were 10⁴, and T cells were 5*10⁴). Afterwards, the gradiently diluted IgFD-36 and IgFD-37 were added for incubation for 24 h at 37° C. LDH content in culture supernatant was detected by a Cytotox-96 nonradioactive cytotoxicity assay kit (Promega). OD values at 490 nm were read by SpectraMax 250. Cytotoxicity (represented by %) was calculated as follows:

% Cytotoxicity=(Experimental−Effector Spontaneous−Target Spontaneous)/(Target Maximum−Target Spontaneous)×100

where, Target Maximum was the LDH content in supernatant where only K562 or PANC-1 cells were lysed.
Target Spontaneous was the LDH content in supernatant where there were only K562 or PANC-1 cells.
Effector Spontaneous was the LDH content in supernatant where there were only effector cells (T cells).

The result was shown in FIG. 14. The heterodimer targeting CD3 and MICA could effectively recruit T cells in PBMCs to produce the corresponding killing effect on MICA positive cells.

### 4.5 Analysis of the Killing Effect of PMBCs on HL-60 Cells Medidated by the Anti-CD3CLL-1 Heterodimer

Peripheral blood of healthy volunteers was collected, and Ficoll-Hypaque(GE Healthcare) was used to separate peripheral blood monouclear cells (PBMCs) via gradient centrifugation. Purified PBMCs were resuspended in RPMI 1640/10% FBS complete medium, and incubated with immobilized anti-CD3 (Clone OKT3, eBiosciences), and 2 μg/mL anti-CD28 (Clone CD28.2, eBiosciences) at 37° C. for 48 h. The activated T cells were then expanded for 10d with 20 U/ml IL2(R&D Systems)

HL-60 cells (a RPMI1640 medium containing 10% FBS) were cultured, and labeled by Green fluorescent cell linker mini kit (Sigma). 10⁴ labeled HL-60 cells were incubated with the above activated T cells at a ratio of 1:5 (HL-60 cells were 10⁴, and T cells were 5*10⁴). Gradiently diluted IgFD-23 was added for incubation for 24 h at 37° C., and then 1%-AAD 7-AAD was added and subjected for FCM. Cells having positive green fluorescence/negative 7-AAD were vital NALM6 cells.

The result was shown in FIG. 15. IgFD-23 can effectively recruit T cells in PBMCs to produce the specific killing effect on HL-60 cells.

TABLE 4
Binding of different heterodimers to cells and promotion
for the killing effect of PBMC on target cells

| | | | Promotion for the specific |
|---|---|---|---|
| | | Binding | killing effect of PBMCs |
| | | to cells | on target cells by |
| Cell | Construct | (Kd, nM) | heterodimers (EC50, pM) |
| Nalm-6 | IgFD-6 | / | 14.95 ± 1.90 |
| | IgFD-7 | / | 11.72 ± 2.12 |
| F98-EGFR | IgFD-8 | 3.118 | 766.2 |
| | IgFD-18 | 0.168 | 2600 |
| | IgFD-19 | 0.771 | 975 |
| | IgFD-21 | / | 12.19 |
| | IgFD-20(Size) | / | 3.34 |
| | IgFD-20(MonoS) | / | 4.23 |
| | IgFD-24 | 6.154 | 198.7 |
| | IgFD-25 | 1.12 | 11 |
| | IgFD-26 | 5.282 | 105.4 |
| | IgFD-28(ProA) | 3.806 | 14.74 |
| | IgFD-28(CH1) | 2.858 | 11.39 |
| | IgFD-29(ProA) | 77.52 | 292.9 |
| | IgFD-29(CH1) | 77.97 | 338 |
| | IgFD-30(ProA) | 130.7 | 651 |
| | IgFD-30(CH1) | 86.34 | 435.3 |
| | IgFD-31 | 5.473 | / |
| | IgFD-32(ProA) | 19.61 | |
| | IgFD-32(CH1) | 12.82 | |
| | IgFD-33 | 2.422 | |
| | IgFD-36 | / | 1496 |
| | Anti-EGFR | 0.3546 | / |
| Jurkat T | IgFD-24 | 11.49 | / |
| | IgFD-25 | 7.723 | |
| | IgFD-26 | 4.228 | |
| | IgFD-28(ProA) | 12.16 | |
| | IgFD-28(CH1) | 10.39 | |
| | IgFD-29(ProA) | 7.416 | |
| | IgFD-29 (CH1) | 6.816 | |
| | IgFD-30(ProA) | 6.742 | |
| | IgFD-30 (CH1) | 7.351 | |
| | IgFD-31 | 4.162 | |
| | IgFD-32(ProA) | 7.135 | |
| | IgFD-32 (CH1) | 8.447 | |
| | IgFD-33 (ProA) | 50.71 | |
| | IgFD-36 (CH1) | 6.118 | |
| PBMC-T | IgFD-8 | 3.741 | / |
| | IgFD-18 | 29.13 | |
| | IgFD-19 | 26.57 | |
| MM1.R | IgFD-22 | / | 593.2 ± 105.79(by LDH) |
| | | | 222.1 ± 60.78(by flow |
| | | | cytometry) |
| HL-60 | IgFD-23 | / | 60.88 ± 7.44 |
| K562 | IgFD-36 | / | 4155 |
| | IgFD-37 | / | 1088 |
| PANC-1 | IgFD-36 | / | 13270 |
| | IgFD-37 | / | 676 |

### Example 5 Measurement of Thermostability

Samples IgFD-6 and IgFD-7 were mixed with freshly prepared thermal shift dye, shift buffer (Protein Thermal Shift™ Dye Kit, Thermo Fisher Scientific, Cat. 4461146) at a ratio recommended by manufacturer, and ViiA™ 7 Real-Time PCR System was used for thermal scanning at 25-99° C. with a heating rate of 0.05° C./s. Tm value was calculated by an “Area under curve(AUC)” analysis model of GraphPad Prism7 software. Each set of data should be obtained by repeated two tests, thus ensuring the repeatability of the result.

The result was shown in Table 5, and IgFD-6 and IgFD-7 had similar Tm values.

TABLE 5
Tm values of protein

| Protein | Tm (° C.) |
|---|---|
| IgFD-6 | 67.52 |
| IgFD-7 | 67.92 |

### Example 6 PK Studies of Heterodimers in Rats

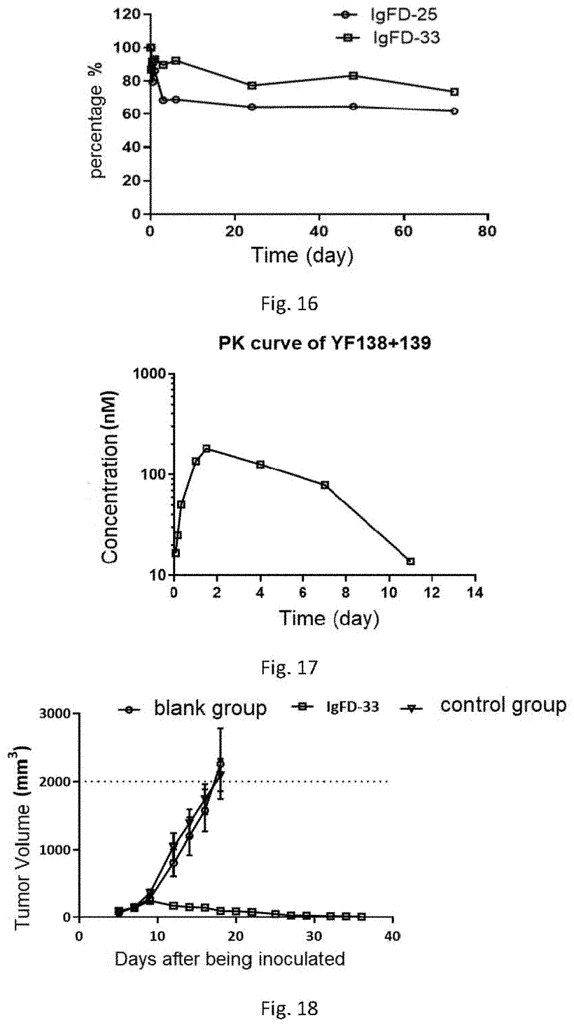

SD male rats (n=3) were intraperitoneally injected (I.P.) with IgFD-33. Heparin-anticoagulated blood was collected from the tail vein at the time as follows: 2 h, 4 h, 8 h, 24 h, 36 h, 4 d, 7 d, 11d and 14 d. After centrifugation, the plasma was collected and stored at −80° C. for further use. The IgFD-33 content in plasma was detected as that described in Example 4.2 (1). The results were shown in FIG. 16. The half-life of IgFD-33 in rats can reach 2.5 d.

### Example 7 In Vivo Efficacy Studies of Heterodimers in a Mouse Model

The ability of IgFD-33 to inhibit tumor mass in tumor-bearing mice was tested in 6-8 week female NCG mice. 5×10⁶ A431 cells and 2.5×10⁶ fresh PBMC were resuspended with 200 μl serum-free medium, and subcutaneously injected into the right flank of mice (DayO), and tumors were measured by callipers. Tumor volume was calculated by the following formula: tumor volume=width*width*length/2. When the tumor size was 50-100 mm³ (Day7), mice were injected with activated 1.0×10⁷ T cells (immobilized anti-CD3 antibody (clone OKT3, eBioscience), 2 μg/mL anti-CD28 antibody (clone CD28.2, eBioscience), and 50 IU/mL recombinant human IL-2 (R&D Systems was used for in vitro activation of PBMCs) on Day8 and Day11; meanwhile, the mice were injected with the heterodimeric antibody IgFD-33 daily on Day7-Day13 (where the blank group is injected with A431 and saline, and control group was injected with A431, fresh PBMC, activated T cells and saline). Body weight was recorded and tumor volume was measured by callipers every other day.

The results were shown in FIG. 17. In comparison to the blank group or control group, the tumor mass of mice inoculated with A431 cells decreased continuously after IgFD-33 administration, no tumor recurrence was observed 35 days after A431 inoculation, indicating that IgFD-33 had a higher in vivo activity.

What is claimed is:

1. A heterodimeric fusion protein, comprising:
a) a first polypeptide chain, comprising a Fab heavy chain and a first Fc chain, wherein the Fab heavy chain is fused to N-terminal of the first Fc chain directly or via a linker;
b) a second polypeptide chain, comprising a Fab light chain and a second Fc chain, wherein the Fab light chain is fused to N-terminal of the second Fc chain directly or via a linker;
wherein, the Fab heavy chain of the first polypeptide chain and the Fab light chain of the second polypeptide chain form a first antigen binding domain; and the first and second Fc chain form an Fc dimerization domain;
wherein, the constant domains CH1 and CL of Fab can be interchanged in two chains.

2. The heterodimeric fusion protein of claim 1, further comprising a second antigen binding domain; preferably, the second antigen binding domain is selected from the followings: scfv, biologically active peptides, Fv, or Fab2.

3. (canceled)

4. The heterodimeric fusion protein of claim 2, wherein the scfv or the biologically active peptide is fused to N-terminal or C-terminal of the first polypeptide chain or the second polypeptide chain directly or via a linker.

5. (canceled)

6. The heterodimeric fusion protein of claim 2, wherein the second antigen binding domain consists of a first biologically active peptide and a second biologically active peptide, and the first biologically active peptide and the second biologically active peptide are fused to N-terminal or C-terminal of the first polypeptide chain and the second polypeptide chain directly or via a linker, respectively.

7. (canceled)

8. (canceled)

9. The heterodimeric fusion protein of claim 2, wherein the heavy chain variable region of the second binding domain Fv is fused to C-terminal of the first polypeptide chain or the second polypeptide chain directly or via a linker; and accordingly, the light chain variable region of the Fv is fused to C-terminal of the second polypeptide chain or the first polypeptide chain directly or via a linker; or the heavy chain variable region of the Fv is fused to N-terminal of the first polypeptide chain or the second polypeptide chain directly or via a linker, and accordingly, the light chain variable of the Fv is fused to N-terminal of the second polypeptide chain or the first polypeptide chain directly or via a linker.

10. (canceled)

11. (canceled)

12. The heterodimeric fusion protein of claim 2, wherein, the heavy chain of the Fab2 is fused to C-terminal of the first polypeptide chain or the second polypeptide chain directly or via a linker; and accordingly, the light chain of the Fab2 is fused to C-terminal of the second polypeptide chain or the first polypeptide chain directly or via a linker; or the heavy chain variable region of the Fab2 is fused to N-terminal of the first polypeptide chain or the second polypeptide chain directly or via a linker, and accordingly, the light chain variable of the Fab2 is fused to N-terminal of the second polypeptide chain or the first polypeptide chain directly or via a linker; wherein further, constant domains CH1 and CL on Fab2 can be interchanged in two chains.

13. (canceled)

14. (canceled)

15. The heterodimeric fusion protein of claim 1, wherein the linker is selected from, but not limited to, a group consisting of the following amino acid sequences: GGSGAKLAALKAKLAALKGGGGS, GGGGSELAALEAELAALEAGGSG, GGGGSGGGGSGGGGS, SGGGGSGGGGSGGGGS, GGSGGSGGGGSGGGG, GGSGGSGGGGSGGGGS, GGSGAKLAALKAKLAALKGGGGS, GGGGSELAALEAELAALEAGGSG, APATSLQSGQLGFQCGELCSASA, ASTKGP, TVAAPSVFIFPP, PNLLGGP, GGGGS, and GGGEAAAKEAAAKEAAAKAGG.

16. A heterodimeric fusion protein, comprising:
a) a first polypeptide chain, comprising: Fc, (L1)n, CH1, L2, and VH from N-terminal to C-terminal sequentially;
b) a second polypeptide chain, comprising: Fc, (L3)n, CL, L4, and VL from N-terminal to C-terminal sequentially;
or
a) a first polypeptide chain, comprising: Fc, (L1)n, CL, L2, and VH from N-terminal to C-terminal sequentially;
b) a second polypeptide chain, comprising: Fc, (L3)n, CH1, L4, and VL from N-terminal to C-terminal sequentially,
wherein, n is 0 or 1; L1, L2, L3 and L4 are linkers; VH and VL form a first antigen binding domain.

17. The heterodimeric fusion protein of claim 16, further comprising a second antigen binding domain; preferably, the second antigen binding domain consists of a scfv or a biologically active peptide, wherein the scfv or the biologically active peptide is fused to N terminal of the first polypeptide chain or the second polypeptide chain directly or via a linker; or the first biologically active peptide and the second biologically active peptide are fused to N-terminal of the first polypeptide chain and the second polypeptide chain directly or via a linker, respectively.

18. (canceled)

19. (canceled)

20. (canceled)

21. The heterodimeric fusion protein of claim 16, wherein L1, L2, L3, and L4 linkers are independently selected from: G, GS, SG, SS, GGS, GSG, SGG, GGG, GGGS, SGGG, GGGGS, GGGGSGS, GGGGSGGS, GGGGSGGGGS, GGGGSGGGGSGGGGS, AKTTPKLEEGEFSEAR, AKTTPKLEEGEFSEARV, AKTTPKLGG, SAKTTPKLGG, AKTTPKLEEGEFSEARV, SAKTTP, SAKTTPKLGG, RADAAP, RADAAPTVS, RADAAAAGGPGS, RADAAAA(G4S)4, SAKTTP, SAKTTPKLGG, SAKTTPKLEEGEFSEARV, ADAAP, ADAAPTVSIFPP, TVAAP, TVAAPSVFIFPP, QPKAAP, QPKAAPSVTLFPP, AKTTPP, AKTTPPSVTPLAP, AKTTAP, AKTTAPSVYPLAP, ASTKGP, ASTKGPSVFPLAP, GENKVEYAPALMALS, GPAKELTPLKEAKVS, and GHEAAAVMQVQYPAS; wherein L1, L2, L3, and L4 may be the same or different.

22. The heterodimeric fusion protein of claim 1, wherein the Fc is a human IgG1 Fc;
preferably, the Fc is a Fc variant; preferably, the Fc variant is free of glycosylation; preferably, the Fc variant comprises an amino acid substitution at position N297;
preferably, the Fc variant comprises one or more amino acid substitution which reduce Fc binding to Fc receptor and/or effector function thereof;
preferably, the Fc variant comprises one or more of E233P, L234V, L235A, delG236, A327G, A330S, and A331S;
preferably, one Fc variant further comprises amino acid substitution S354C and T366W, and the other further comprises amino acid substitution Y349C, T366S, L368A and Y407V.

23. (canceled)

24. (canceled)

25. (canceled)

26. (canceled)

27. (canceled)

28. (canceled)

29. The heterodimeric fusion protein of claim 2, wherein the heterodimeric fusion protein may bind to one or a combination of the following antigens: CD3, CD16, CD2, CD28, CD25, NKG2D, NKp46, BCMA, CLL-1, EpCAM, CD19, CCR5, EGFR, HER2, HER3, HER4, EGF4, PSMA, CEA, MUC-1(Mucin), MUC-2, MUC-3, MUC-4, MUC-5_{AC}, MUC-5_{B}, MUC7, βhCG, Lewis-Y, CD20, CD33, CD30, CD16A, B7-H3, CD123, gpA33, P-Cadherin, GPC3, CLEC12A, CD32B, TROP-2, ganglioside GD3, 9-O-Acetyl-GD3, GM2, Globo H, fucosyl GM1, Poly SA, GD2, Carboanhydrase IX (MN/CA IX), CD44v6, Sonic Hedgehog (Shh), Wue-1, Plasma Cell Antigen, (membrane-bound) IgE, Melanoma Chondroitin Sulfate Proteoglycan (MCSP), CCR8, TNF-alpha precursor, STEAP, mesothelin, A33 Antigen, Prostate Stem Cell Antigen (PSCA), Ly-6, desmoglein 4, E-cadherin neoepitope, Fetal Acetylcholine Receptor, CD25, CA19-9 marker, CA-125 marker and Muellerian Inhibitory Substance (MIS) Receptor type II, sTn (sialylated Tn antigen; TAG-72), FAP (fibroblast activation antigen), endosialin, EGFRvIII, LG, SAS, CD63.

30. The heterodimeric fusion protein of claim 2, wherein the heterodimeric fusion protein may bind to antigens consisting of the following antigen pairs: CD3 and CD19; CD3 and CD20; CD3 and BCMA; CD3 and CLL-1; CD3 and EGFR; CD3 and HER2; CD3 and MIC-A; CD3 and CEA; CD3 and PSMA; CD3 and EpCAM;
preferably, the heterodimeric fusion protein comprise the following pairs of two polypeptide chains: SEQ ID No:2 and SEQ ID No:4; SEQ ID No:6 and SEQ ID No:8; SEQ ID No:20 and SEQ ID No:22; SEQ ID No:24 and SEQ ID No:26; SEQ ID No:92 and SEQ ID No:94; SEQ ID No:96 and SEQ ID No:98; SEQ ID No:100 and SEQ ID No:102; SEQ ID No:10 and SEQ ID No: 12; SEQ ID No: 14 and SEQ ID No: 16; SEQ ID No: 18 and SEQ ID No: 12; SEQ ID No:24 and SEQ ID No:28; SEQ ID No:30 and SEQ ID No:32; SEQ ID No:30 and SEQ ID No:34; SEQ ID No:36 and SEQ ID No:38; SEQ ID No:44 and SEQ ID No:46; SEQ ID No:48 and SEQ ID No:50; SEQ ID No:44 and SEQ ID No:52; SEQ ID No:54 and SEQ ID No:56; SEQ ID No:30 and SEQ ID No:58; SEQ ID No:60 and SEQ ID No:62; SEQ ID No:64 and SEQ ID No:66; SEQ ID No:68 and SEQ ID No:70; SEQ ID No:72 SEQ ID No:74; SEQ ID No:76 and SEQ ID No:78; SEQ ID No:80 and SEQ ID No:82; SEQ ID No:84 and SEQ ID No:86; SEQ ID No:88 and SEQ ID No:90; SEQ ID No: 104 and SEQ ID No: 106; SEQ ID No: 108 and SEQ ID No: 110; SEQ ID No: 112 and SEQ ID No: 114; SEQ ID No: 116 and SEQ ID No: 118; SEQ ID No:30 and SEQ ID No:40; SEQ ID No:30 and SEQ ID No:42; SEQ ID No: 122 and SEQ ID No: 124.

31. (canceled)

32. (canceled)

33. (canceled)

34. (canceled)

35. (canceled)

36. (canceled)

37. (canceled)

38. (canceled)

39. (canceled)

40. (canceled)

41. A polynucleotide, encoding the heterodimeric fusion protein of claim 1.

42. A vector, in particular an expression vector, comprising the polynucleotide of claim 41.

43. A host cell, comprising:
an expression vector, comprising a polynucleotide encoding the first polypeptide chain, and
an expression vector, comprising a polynucleotide encoding the second polypeptide chain;
the first polypeptide chain comprising a Fab heavy chain and a first Fc chain, wherein the Fab heavy chain is fused to N-terminal of the first Fc chain directly or via a linker;
the second polypeptide chain comprising a Fab light chain and a second Fc chain, wherein the Fab light chain is fused to N-terminal of the second Fc chain directly or via a linker;
wherein, the Fab heavy chain of the first polypeptide chain and the Fab light chain of the second polypeptide chain form a first antigen binding domain; and the first and the second Fc chain form an Fc dimerization domain.

44. A preparation method of the heterodimeric fusion protein of claim 1, comprising the following steps:
1) transiently transfecting a mammalian host cell with the followings:
an expression vector, comprising a polynucleotide encoding the first polypeptide chain, and
an expression vector, comprising a polynucleotide encoding the second polypeptide chain;
culturing the mammalian host cell under conditions where the expression of the heterodimeric fusion protein is allowed; and
collecting the heterodimeric fusion protein secreted from culture supernatant.

45. A pharmaceutical composition, comprising the heterodimeric fusion protein of claim 1.

46. A method for treating a cancer, an autoimmune disease or virus infection in a subject in need thereof; wherein the method comprises a step of administering an effective amount of the composition to the subject; and the composition comprises the heterodimeric fusion protein of claim 1 in a pharmaceutically acceptable form.

47. A polynucleotide, encoding the heterodimeric fusion protein of claim 16.
